(19) United States
(12) Patent Application Publication
(10) Pub. No.: US 2026/0315625 A1
(43) Pub. Date: Oct. 15, 2026

(54) ALLOTOPIC EXPRESSION OF MTDNA GENES

(71) Applicant: SENS RESEARCH FOUNDATION, Mountain View, CA (US)

(72) Inventors: Amutha BOOMINATHAN, Mountain View, CA (US); Matthew S. O' CONNOR, Mountain View, CA (US); Caitlin J. LEWIS, Mountain View, CA (US); Bhavna DIXIT, Mountain View, CA (US)

(73) Assignee: SENS RESEARCH FOUNDATION, Mountain View, CA (US)

(21) Appl. No.: 19/119,098

(22) PCT Filed: Oct. 6, 2023
(86) PCT No.: PCT/US2023/076302

§ 371 Date: Apr. 7, 2025

Related U.S. Application Data
(60) Provisional application No. 63/414,406, filed on Oct. 7, 2022.

(51) Int. Cl. A61K 48/00; A61K 38/43; A61P 25/00; C12N 15/86

(52) U.S. Cl. CPC A61K 48/0058; A61K 38/43; A61P 25/00; C12N 15/86; C12N 2710/10043; C12N 2710/16643; C12N 2740/15043; C12N 2750/14143

(57) ABSTRACT

Mitochondrial DNA (mtDNA) in humans is a 16,569 base pair double-stranded circular DNA that encodes 13 vital proteins of the electron transport chain. Embodiments of the invention include allotopic expression of mtDNA genes to compensate for a functional deficiency arising from underlying mtDNA damage or mutations. In allotopic expression, genes that are normally expressed only from the mitochondrial genome are modified for expression using nucleo-cytosolic transcription and machinery. The methods can be used therapeutically to treat diseases caused by mitochondrial DNA mutations. The methods can also be used to slow the aging process and/or reduce effects of aging.

### RELATED APPLICATIONS

This application claims priority to U.S. provisional patent application No. 63/414,406 filed on Oct. 7, 2022. The contents of the aforementioned application are incorporated herein by reference.

### FIELD OF THE INVENTION

The invention relates to the field of molecular genetics and more specifically, to the expression of mitochondrial genes in human cells to treat mitochondrial disorders and counteract aging processes and phenotypes.

### BACKGROUND

A mitochondrion is a double-membrane-bound organelle found in most eukaryotic organisms. Although most of a cell's DNA is contained in the cell nucleus, the mitochondrion has its own genome (“mitogenome”) that is similar to bacterial genomes. Mitochondrial proteins (i.e., proteins transcribed from mitochondrial DNA) vary depending on the tissue and the species. In humans, 615 distinct types of proteins have been identified from cardiac mitochondria, whereas in rats, 940 proteins have been reported. The mitochondrial proteome is thought to be dynamically regulated.

Mitochondria use aerobic respiration to generate most of the cell's supply of adenosine triphosphate (ATP), which is subsequently used throughout the cell as a source of energy. Oxidative phosphorylation (OXPHOS), the Krebs's cycle, the urea cycle, heme biosynthesis and fatty acid oxidation take place within the mitochondria. In addition to supplying cellular energy, mitochondria are involved in signaling, cellular differentiation, and cell death, as well as maintaining control of the cell cycle and cell growth. Mitochondrial biogenesis is in turn temporally coordinated with these cellular processes. Mitochondria have been implicated in several human disorders and conditions, such as mitochondrial diseases, cardiac dysfunction, heart failure and autism.

The majority of mitochondrial proteins are encoded by the nuclear genome, translated in the cytosol, and imported into the mitochondrion. However, 13 core subunits of respiratory complexes are encoded by the reduced mitochondrial genome and synthesized within the mitochondrial matrix. Mutations in these 13 genes (or their associated non-protein-coding genes) tend to be especially severe, as all 13 proteins are core subunits of the oxidative phosphorylation pathway. Any disruption to subunit structure, stability, or function can have major biochemical and physiological consequences. For example, mtDNA mutations that cause structural changes in OXPHOS subunits disrupt the electron transfer relay, resulting in inefficient energy production. Inefficient transfer can generate superoxide byproducts, resulting in increased reactive oxygen species (ROS) and reactive nitrogen species (RNS), causing a chronic state of cellular stress.

There are several known mitochondrial diseases and disorders that are caused by pathogenic point mutations of mitochondrial DNA (mtDNA), one-third of which are located in coding genes. Primary defects in mitochondrial function generally present clinical problems in tissues that have high energy requirements, such as retina, heart, muscle, kidney, pancreas and liver. Their incidence is estimated at 1 in 5,000 live births. Thus, mitochondrial pathologies are considered among the most common genetically determined diseases and are a major health issue since they remain inaccessible to both curative and palliative therapies.

Mitochondrial dysfunction is also a hallmark of aging and cellular senescence. There are changes in mitochondrial metabolism with aging that are linked to changes in mitochondrial organization. The dynamic equilibrium between fusion and fission is essential for healthy mitochondrial function. In senescent cells, fusion exceeds fission, and large mitochondria form that contribute to cell senescence. The fission process is coupled with ER-microtubule function; therefore, interaction of mt-ER-Lysosome affects the fission process. There is evidence of decreased senescence with exercise and findings indicate that mechanical forces alter MT-ER junction protein complexes through unknown processes in the fission and fusion functions. Furthermore, there is evidence of a decline in mitochondrial quality and copy number and an increasing incidence of mtDNA mutations with age, which are consequently implicated in cellular senescence and age-related organismal decline.

With advanced age, mitochondrial DNA volume, integrity and functionality decrease due to accumulation of mutations and oxidative damage induced by reactive oxygen species (ROS). In aged subjects, mitochondria are characterized by impaired function such as lowered oxidative capacity, reduced oxidative phosphorylation, decreased ATP production, significant increase in ROS generation, and diminished antioxidant defense. Mitochondrial biogenesis declines with age due to alterations in mitochondrial dynamics and inhibition of mitophagy, an autophagy process that removes dysfunctional mitochondria. Age-dependent abnormalities in mitochondrial quality control further weaken and impair mitochondrial function. In aged tissues, enhanced mitochondria-mediated apoptosis contributes to an increase in the percentage of apoptotic cells.

There are currently no cures for mitochondrial disorders. The few treatment options available are generally limited to managing symptoms. Conventional treatments include nutraceutical supplements, and lifestyle interventions, such as dietary restriction and exercise.

Allotopic expression has been proposed to treat these mitochondrial disorders. In allotopic expression, a wild-type (i.e., healthy) copy of a mutated gene is introduced in the genome. Normal copies of the gene product are imported into mitochondria from the cytosol. Currently, a number of mitochondrial genes have been successfully recoded and nuclearly expressed in yeast. However, efforts at allotopic expression in mammalian cells have been generally unsuccessful.

Accordingly, methods for allotopic expression must be improved before they can be used therapeutically in humans. There are several hurdles to allotopic expression of a mitochondrial gene including:
a) a different codon dictionary used by the mitochondrial and nuclear genomes,
b) different codon preferences between mitochondrial and nuclear-cytosolic translation systems, and
c) the provision of an import signal to ensure that the newly translated protein in the cytosol is successfully imported into mitochondria.

Because of these issues and others, further studies are necessary to elucidate the steps of nuclear gene expression, import into mitochondria, processing, and functional integration of the allotopically expressed polypeptides into mitochondrial protein complexes.

There is a need for reliable methods of allotopic expression in mammalian cells. The present invention overcomes the problems of conventional approaches. The methods described herein can be used therapeutically to treat mitochondrial diseases and age-related conditions.

### SUMMARY OF THE INVENTION

The inventions described and claimed herein have many attributes and embodiments including, but not limited to, those set forth or described or referenced in this brief summary. The inventions described and claimed herein are not limited to, or by, the features or embodiments identified in this summary, which is included for purposes of illustration only and not restriction.

The invention provides means, including compositions and methods, which enable mitochondrial importation at enhanced efficiency and stability compared to conventional techniques. The means of the invention enable a targeted localization of the mRNA to the mitochondrial surface.

Accordingly, embodiments include methods of introducing a modified gene product into a cell by, for example, plasmid or viral vector, as an RNA or cDNA therapeutic or as a therapeutic protein. In aspects, the gene product is expressed from the nucleus (i.e., genome integration). In other aspects, the gene product is provided as a non-integrating vector, or as cDNA or mRNA, with expression/regulatory elements appropriate for the nucleic acid type. In still other aspects, a therapeutic protein is administered to a subject (i.e., direct to tissue).

Embodiments include methods of allotopic expression of a gene in a cell. The methods can include steps of (a) identifying a mitochondrial gene in the cell that has one or more mutations, (b) expressing a wild-type version of the mitochondrial gene in the nucleus of the cell and (c) importing a gene product of the wild-type version of the mitochondrial gene into mitochondria of the cell. In embodiments, the cell is a mammalian cell such as a human cell.

In embodiments, the gene product is an oxidative phosphorylation (OXPHOS) complex subunit translated on the mitochondrial DNA.

In embodiments, one or more codon sequences are incorporated into the wild-type version of the mitochondrial gene. The methods described herein can use codon optimization, translational slow-down and/or ribosome stalling.

In embodiments, an N-terminal matrix export signal is incorporated into the wild-type version of the mitochondrial gene. In embodiments, one or more codon sequences are introduced into the wild-type version of the mitochondrial gene to restore natural orientation of transmembrane helices of the gene product into the mitochondrial inner membrane.

In embodiments, methods of allotopic expression described herein are used therapeutically to improve mitochondrial function, for example, to treat a mitochondrial dysfunction, a senescence-associated disease or disorder or to prevent or slow the aging process.

Embodiments also include methods of reducing the hydrophobicity of the coding sequences to improve import and function of allotopic mtDNA proteins. The methods can include steps of (a) identifying non-deleterious mutations and to assess the resulting hydrophobicity alterations and (b) evaluating the three-dimensional (3D) structural impact of the mutations.

Embodiments include methods of administering a nucleic acid construct that encodes a therapeutic mitochondrial protein. In aspects, the construct is integrated into the genome. In aspects, the construct does not integrate into the genome (e.g., expressed from an episome). In aspects, the gene product is imported into mitochondria.

Embodiments include administration of nucleic acids encoding therapeutic mitochondrial protein. In aspects, the nucleic acids are RNA.

Embodiments include allotopic administration of a therapeutic mitochondrial protein.

In embodiments, a mitochondrial targeting sequence (MTS) identified in Table 1 (i.e., SEQ ID NO: 1-29) is paired with a nuclear-encoded mitochondrial protein of respiratory complex I.

In embodiments, a mitochondrial targeting sequence (MTS) identified in Table 2 (i.e., SEQ ID NO: 30-48) is paired with a nuclear-encoded mitochondrial protein of respiratory complex V.

In embodiments, a mitochondrial targeting sequence (MTS) identified in Table 3 (i.e., SEQ ID NO: 49-56) is paired with a nuclear-encoded mitochondrial protein of respiratory complex IV.

In embodiments, a mitochondrial targeting sequence (MTS) identified in Table 4 (i.e., SEQ ID NO: 57-63) is paired with a nuclear-encoded mitochondrial protein of respiratory complex III.

In embodiments, a mitochondrial targeting sequence (MTS) identified in Table 5 (i.e., SEQ ID NO: 64-118) is paired with a transmembrane domain-containing nuclear encoded protein of the inner membrane.

In embodiments, a mitochondrial targeting sequence (MTS) identified in Table 6 (i.e., SEQ ID NO: 119-280) is paired with a nuclear encoded protein of the inner membrane lacking transmembrane (TM) domains.

In embodiments, therapeutic nucleic acids or protein are encapsulated in a liposome, nanoparticle or other pharmaceutically acceptable carrier.

In embodiments, a viral vector is used in the step of expressing a nucleic acid construct that encodes a therapeutic mitochondrial protein. In aspects, the viral vector is a lentiviral vector, a herpes simplex virus (HSV) vector, an adenoviral vector or an adeno-associated viral (AAV) vector.

### BRIEF DESCRIPTION OF THE DRAWINGS

The accompanying drawings illustrate aspects of the present invention. In such drawings:

FIG. 1A is a depiction of steps in allotopic expression.

FIG. 1B is a depiction of pCMV constructs according to embodiments of the invention.

FIG. 1C is a bar graph showing the levels of OND1 mRNA expression in ND1 null cell lines for each construct (relative to GAPDH).

FIG. 1D is a bar graph showing the levels of OND1 mRNA expression in ND1 null cell lines for each construct (relative to COX10).

FIG. 2A is depiction of pCMV and pCAG constructs according to embodiments of the invention.

FIG. 2B is a bar graph showing the levels of OND1 mRNA expression in ND1 null cell lines for each construct (relative to GAPDH).

FIG. 2C is a bar graph showing the levels of OND1 mRNA expression in ND1 null cell lines for each construct (relative to COX10).

FIG. 3A is depiction of constructs with Hexapeptide and OXA1L sequences according to embodiments of the invention.

FIG. 3B is a bar graph showing the levels of OND1 mRNA expression in ND1 null cell lines for each construct (relative to GAPDH).

FIG. 3C is a bar graph showing the levels of OND1 mRNA expression in ND1 null cell lines for each construct (relative to COX10).

FIG. 4A is a depiction of constructs with MPCP sequences according to embodiments of the invention.

FIG. 4B is a bar graph showing the levels of OND1 mRNA expression in ND1 null cell lines for each construct (relative to GAPDH).

FIG. 4C is a bar graph showing the levels of OND1 mRNA expression in ND1 null cell lines for each construct (relative to COX10).

FIG. 5A is a depiction of constructs with ABCBA sequences according to embodiments of the invention.

FIG. 5B is a bar graph showing the levels of OND1 mRNA expression in ND1 null cell lines for each construct (relative to GAPDH).

FIG. 5C is a bar graph showing the levels of OND1 mRNA expression in ND1 null cell lines for each construct (relative to COX10).

FIG. 6A is a depiction of constructs with triple protein tags according to embodiments of the invention.

FIG. 6B is a bar graph showing the levels of OND1 mRNA expression in ND1 null cell lines for each construct (relative to GAPDH).

FIG. 6C is a bar graph showing the levels of OND1 mRNA expression in ND1 null cell lines for each construct (relative to COX10).

FIG. 7A is graphical depiction of mesohydrophobicity versus local hydrophobicity of transmembrane region 1 of ATP6.

FIG. 7B is graphical depiction of mesohydrophobicity versus local hydrophobicity of transmembrane region 1 of ATP6.

FIG. 8A is a bar graph showing the levels of oATP6 mRNA expression in ATP6 mutant cell lines for different constructs to COX10.

FIG. 8B is a bar graph showing the levels of oATP6 mRNA expression in ATP6 mutant cell lines for different constructs to GAPDH.

FIG. 9 is an image of SDS PAGE of mitochondrial fractions with a comparison of anti-FLAG and anti-ACONITASE expression.

FIG. 10A depicts the cleaved and uncleaved COX2 protein.

FIG. 10B is an SDS PAGE showing stable expression in COX2 site-directed mutagenesis constructs.

FIG. 11A shows mRNA levels for different versions of codon-optimized allotopic COX2 gene in comparison to GAPDH.

FIG. 11B shows SDS PAGE profiles for different versions of codon-optimized allotopic COX2 in a COX2 null cell line upon stable selection.

FIG. 12 shows a plasmid sequence of a COX2 variant according to embodiments.

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

The term “allotopic expression” or “AE” refers to expression of genes normally expressed only from the mitochondrial genome using nucleo-cytosolic machinery. Biomedically engineered AE has been suggested as a possible tool in gene therapy to treat certain mitochondria-related diseases.

The term “mitochondrial diseases” refers to chronic, genetic, often inherited disorders that occur when mitochondria fail to produce enough energy for the body to function properly. Mitochondrial diseases can affect almost any part of the body, including the cells of the brain, nerves, muscles, kidneys, heart, liver, eyes, ears or pancreas. Mitochondrial diseases can be caused by mitochondrial DNA (mtDNA) disorders or nuclear DNA (nDNA) disorders.

Mitochondrial DNA (mtDNA) disorders include, for example, Leigh syndrome, leukodystrophy w/complex II deficiency, cardiomyopathy & encephalopathy (complex I deficiency), optic atrophy and ataxia (complex II deficiency), hypokalemia and lactic acidosis, hepatopathy & ketoacidosis, hypertrophic cardiomyopathy, liver failure, renal tubulopathy (w/complex III deficiency) and encephalopathy (w/complex V deficiency), autosomal progressive external ophthalmoplegia, mitochondrial neurogastrointestinal encephalomyopathy, Alpers-Huttenlocher syndrome, ataxia neuropathy syndromes, infantile myopathy/spinal muscular atrophy and hypotonia.

The term “primary mitochondrial disorders” refers to a clinically heterogeneous group of disorders that arise as a result of dysfunction of the mitochondrial respiratory chain. The mitochondrial respiratory chain is the essential final common pathway for aerobic metabolism. Tissues and organs that are highly dependent on aerobic metabolism are usually most affected by mitochondrial disorders. Many genetic and non-genetic disorders involve mitochondrial mechanisms as a secondary feature. However, “primary mitochondrial disorders” are considered to be known or presumed genetic disorders caused by pathogenic variants in genes coding for the mitochondrial respiratory chain and related proteins.

Nuclear DNA (nDNA) disorders include, for example, Leigh syndrome, leukodystrophy w/complex II deficiency, cardiomyopathy & encephalopathy (complex I deficiency), optic atrophy & ataxia (complex II deficiency), hypokalemia & lactic acidosis (complex III deficiency), hepatopathy and ketoacidosis, cardiomyopathy and encephalopathy, leukodystrophy & renal tubulopathy, hypertrophic cardiomyopathy, liver failure, renal tubulopathy (w/complex III deficiency), encephalopathy (w/complex V deficiency), coenzyme Q10 deficiency, Barth syndrome, autosomal progressive external ophthalmoplegia, mitochondrial neurogastrointestinal encephalomyopathy, Alpers-Huttenlocher syndrome, ataxia neuropathy syndromes, infantile myopathy/spinal muscular atrophy, hypotonia, reversible hepatopathy, myopathy with cataract and combined RC deficiency.

The term “secondary mitochondrial dysfunction” refers to any abnormal mitochondrial function other than a primary mitochondrial disorder. Secondary mitochondrial dysfunction (SMD) can be caused by genes encoding neither function nor production of the oxphos proteins and accompanies many hereditary non-mitochondrial diseases. Secondary mitochondrial dysfunction can also be due to nongenetic causes such as environmental factors. Secondary mitochondrial dysfunction is seen in many different genetic disorders, including ethylmalonic aciduria (caused by mutation of ETHE1), Friedreich ataxia (FXN), hereditary spastic paraplegia 7 (SPG7), and Wilson disease (ATP7B), and is also seen as part of the aging process.

The term “mutation” refers to a change that occurs in our DNA sequence as the result of chemical mutagens such as reactive byproducts of cellular metabolism, environmental factors such as UV light and cigarette smoke, or due to DNA replication errors arising from inherent or acquired deficiency of replication machinery.

A mitochondrial mutation may be described as a non-silent point mutation in one or more mtDNA genes encoding subunits of the respiratory chain. A mutation may also consist of base insertions or deletions, or deletion or duplication of mtDNA genomic regions. Mitochondrial mutation, in this context, also refers to a deficiency in mtDNA copy number which inhibits compensation of a deleterious phenotype.

The term “mitochondrial deficiency” refers to a deficiency characterized by inadequate mtDNA copy number, reduced or absent expression of mtDNA subunits, or expression of faulty mtDNA subunits, and subsequent state of inadequate energy metabolism of a cell (or tissue or organ system or individual). A mitochondrial deficiency can also be caused by a deficiency in mtDNA copy number which leads to a phenotypic deficiency.

The term “inadequate energy metabolism” refers to a condition of reduced mitochondrial function such as inefficient electron transport, ATP synthesis, or an alteration of mitochondrial membrane potential.

The term “wild type” refers to the phenotype of the typical form of a species as it occurs in nature.

The term “deleterious mutation” refers to a mutation in which the protein product of a gene is not produced, is produced and does not function, or is produced and interferes with normal function. Equally, mutations in regulatory elements can be deleterious if the regulatory function is impaired. These mutations can arise from single base changes or more extensive insertions, deletions or frame shifts. Mutations in protein coding genes and regulatory genes can also be neutral. A base change in a protein coding gene that does not alter the amino acid sequence of the protein is termed a synonymous change. These are commonly neutral. A base change that alters the amino acid sequence is likely to be deleterious but can be neutral.

The term “conservative substitution” refers to the replacement of an amino acid residue by another, biologically similar residue. Examples of conservative substitutions include the substitution of one hydrophobic residue such as isoleucine, valine, leucine, alanine, cysteine, glycine, phenylalanine, proline, tryptophan, tyrosine, norleucine or methionine for another, or the substitution of one polar or charged residue for another residue with similar polarity or charge, such as the substitution of arginine for lysine, glutamic acid for aspartic acid, or glutamine for asparagine, and the like. Neutral hydrophilic amino acids that can be substituted for one another include asparagine, glutamine, serine and threonine. The term “conservative substitution” also includes the use of a substituted amino acid in place of an unsubstituted parent amino acid.

The term “senescence” refers to gradual deterioration of functional characteristics in living organisms. Cellular senescence is often defined as a stress-induced, durable cell cycle arrest of previously replication-competent cells. The effects of senescent cells can be thought of as beneficial or detrimental with regard to host physiology and disease, although in some contexts, senescent cells affect a disease state in a complex manner both promoting and opposing certain conditions.

The term “senescence-associated disease or disorder” refers to an ailment that is associated with age and can include, for example, atherosclerosis, osteoarthritis, osteoporosis, hypertension, arthritis, cataracts, cancer, Alzheimer's disease, chronic obstructive pulmonary disease (COPD) and idiopathic pulmonary fibrosis. Other ailments (including age-related conditions) associated with age or senescence include hair graying, sarcopenia, adiposity, neurogenesis, fibrosis and glaucoma.

Still other ailments associated with age or senescence include cardiovascular disease (e.g., atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, and stroke). A senescence-associated disease or disorder can also be an inflammatory or autoimmune disease or disorder (e.g., osteoarthritis, osteoporosis, oral mucositis, inflammatory bowel disease or kyphosis). A senescence-associated disease or disorder can also be a neurodegenerative disease (e.g., Alzheimer's disease, Parkinson's disease, Huntington's disease, dementia, mild cognitive impairment or motor neuron dysfunction). A senescence-associated disease or disorder can also be a metabolic disease (e.g., diabetes, diabetic ulcer, metabolic syndrome or obesity). A senescence-associated disease or disorder can also be a pulmonary disease (e.g., pulmonary fibrosis, chronic obstructive pulmonary disease, asthma, cystic fibrosis, emphysema, bronchiectasis or age-related loss of pulmonary function). A senescence-associated disease or disorder can also be an eye disease or disorder (e.g., macular degeneration, glaucoma, cataracts, presbyopia or vision loss). A senescence-associated disease or disorder can also be renal disease, renal failure, frailty, hearing loss, muscle fatigue, skin conditions, skin wound healing, liver fibrosis, pancreatic fibrosis, oral submucosa fibrosis or sarcopenia. A senescence-associated disease or disorder can also be a dermatological disease or disorder (e.g., eczema, psoriasis, hyperpigmentation, nevi, rashes, atopic dermatitis, urticaria, diseases or disorders related to photosensitivity or photoaging).

The term “oxidative phosphorylation” or “OXPHOS” refers to the process by which ATP synthesis is coupled to the movement of electrons through the mitochondrial electron transport chain and the associated consumption of oxygen. In eukaryotes, this takes place inside mitochondria. In eukaryotes, redox reactions are catalyzed by a series of protein complexes within the inner membrane of the cell's mitochondria. These linked sets of proteins are called the electron transport chain. In eukaryotes, five main protein complexes are involved that use a variety of electron donors and acceptors.

More than 70 different polypeptides interact on the inner mitochondrial membrane to form the electron transport chain, also known as the respiratory chain. Thirteen essential subunits are encoded by mitochondrial DNA (mtDNA) located within mitochondria, along with the ribosomal and transfer RNAs required for intra-mitochondrial protein synthesis. The remaining respiratory chain polypeptides, and proteins essential for the assembly of the respiratory chain, mitochondrial structure, and the maintenance and expression of mtDNA are encoded by the nuclear genome (nDNA).

The term “codon optimization” refers to experimental approaches designed to improve the codon composition of a recombinant gene based on various criteria without altering the amino acid sequence. This is possible because most amino acids are encoded by more than one codon.

The term “promoter” refers to a sequence of DNA to which proteins bind to initiate transcription of a single RNA transcript from the DNA downstream of the promoter. The RNA transcript may encode a protein (mRNA), or can have a function in and of itself, such as tRNA or rRNA. Promoters are located near the transcription start sites of genes, upstream on the DNA (towards the 5′ region of the sense strand) and contain usually about 100-1000 base pairs. The CMV promoter is a strong synthetic promoter frequently used to drive high levels of gene expression in mammalian expression vectors.

The term “pCMV” or “pCMV-Script vector” refers to a promoter sequence that is derived from a high-copy-number pUC-based plasmid and is designed to allow protein expression in mammalian systems. Mammalian expression is driven by the human cytomegalovirus (CMV) immediate early promoter to promote constitutive expression of cloned inserts in a wide variety of cell lines.

The term “5′ untranslated region,” “5′ UTR,” “leader sequence,” “transcript leader” or “leader RNA” refers to the region of a messenger RNA (mRNA) that is directly upstream from the initiation codon. This region is important for the regulation of translation of a transcript by differing mechanisms in viruses, prokaryotes and eukaryotes. While called untranslated, the 5′ UTR or a portion of it is sometimes translated into a protein product. This product can then regulate the translation of the main coding sequence of the mRNA. In many organisms, however, the 5′ UTR is completely untranslated, instead forming complex secondary structure to regulate translation.

The term “canonical mitochondrial localization signal,” “MLS,” “mitochondrial targeting sequence” or “MTS” refers to a short peptide, about 15-70 amino acids long, bearing positively charged basic residues, that directs the transport of a protein to the mitochondria. The term “N-terminal mitochondrial targeting sequence” refers to an MTS on the amino terminal portion of a protein which may interact with mitochondrial import machinery to aid in translocation of soluble proteins to the matrix.

The term “translational stalling” or “ribosome stalling” refers to a situation in ribosomes moving along the mRNAs slow down or stall. Stalling can happen when, for example, the mRNA sequence utilizes codons which rely on low-abundance tRNA species.

The term “OXA1L” or “mitochondrial inner membrane protein OXA1L” refers to a protein that in humans is encoded by the OXA1L gene located on 14q11.2. The C-terminus of this protein interacts with mitochondrial ribosomes and helps insert both mitochondrial and nuclear produced protein termini into or translocate across the inner membrane of the mitochondria from the matrix.

The term “PUMILIO,” “PUM” or “Human Pumilio protein” refers to sequence specific RNA-binding proteins that regulate protein expression. Members of the Pumilio family of proteins (Puf) regulate translation and mRNA stability in a variety of eukaryotic organisms. Pumilio family members are characterized by the presence of eight tandem copies of an imperfectly repeated 36 amino acids sequence motif (i.e., the Pumilio repeat) surrounded by a short N- and C-terminal conserved region. Pumilio interaction motifs are defined in several species.

The term “treating” or “treatment” refers to one or more of (1) inhibiting the disease, condition or disorder in an individual who is experiencing or displaying the pathology or symptomatology of the disease, condition or disorder (i.e., arresting further development of the pathology and/or symptomatology); and (2) ameliorating the disease condition or disorder in an individual who is experiencing or displaying the pathology or symptomatology of the disease, condition or disorder (i.e., reversing the pathology and/or symptomatology) such as decreasing the severity of disease.

The term “administration” refers to the introduction of an amount of a predetermined substance into a patient by a certain suitable method. The compositions disclosed herein may be administered via any of the common routes, as long as it is able to reach a desired tissue, for example, inhaling, intraperitoneal, intravenous, intramuscular, subcutaneous, intradermal, oral, topical, intranasal, intrapulmonary, or intrarectal administration.

Compositions include a therapeutic peptide encoded by a DNA or RNA sequence and an acceptable carrier. The therapeutic peptide can be contained within a delivery vehicle. The two main approaches used to deliver the therapeutic genetic material are (a) synthetic and (b) viral delivery vehicles. Synthetic vectors include, for example, encapsulation in liposomes, nanoparticles, cyclodextrins, and microvesicles. Viruses include, for example, AAV and retroviral vectors.

The term “liposome” refers to a spherical vesicle having at least one lipid bilayer (i.e., an aqueous solution core surrounded by a hydrophobic membrane). Liposomes can be prepared by disrupting biological membranes (such as by sonication). Liposomes are formed when phospholipids and their derivatives are dispersed in water. Upon dispersion in water the phospholipids form closed vesicles called “liposomes,” which are characterized by lipid bilayers encapsulating an aqueous core. Liposomes have therapeutic applications including delivering drugs to target cells after systemic administration. Liposomes can be modified by the incorporation of polyethylene glycol or other hydrophilic polymers (e.g., a PEG liposome where one or more of the constituent lipids is modified by attachment of PEG). Liposomes can also be modified to target particular cell types by incorporating targeting factors (e.g., “targeting ligands”) for particular cell types. Examples include asialoglycoprotein, folate, transferrin, antibodies, etc.

The term “stable formulation” or “stable pharmaceutical formulation” refers to a formulation which preserves its physical stability/identity/integrity and/or chemical stability/identity/integrity and/or biological activity/identity/integrity during manufacturing, storage, transportation, and application. Various analytical techniques for evaluating virus stability are available in the art and reviewed in Felix A. Rey and Shee-Mei Lok (2018) “Common Features of Enveloped Viruses and Implications for Immunogen Design for Next-Generation Vaccines,” Cell 172, pp 1319-1334 and Guy Ungerechts et al. (2016) “Moving oncolytic viruses into the clinic: clinical-grade production, purification, and characterization of diverse oncolytic viruses,” Nature: Molecular Therapy—Methods & Clinical Development 3, 16018. Stability can be evaluated by, for example, without limitation, storage at selected climate conditions for a selected time period, by applying mechanical stress such as shaking at a selected shaking frequency for a selected time period, by irradiation with a selected light intensity for a selected period of time, or by repetitive freezing and thawing at selected temperatures.

As used herein, a “virus” may encompass any chemical or biochemical component portion of a virus, including viral component preparations, virus-like particles, viral vectors, nanoparticles that are enveloped in lipid, a related particle (e.g. prion), or the like and it need not be infective or capable of self-replication. A viral particle can comprise nanoparticles that are enveloped with lipid.

The term “viral vector” refers to a viral genome that has been adapted into a plasmid-based technology and modified for safety through the removal of many essential genes and the separation of the viral components. The use of viral vectors is a means of gene transfer to modify a specific cell type or tissue and can be manipulated to express therapeutic genes.

The term “adeno-associated virus” or “AAV” refers to small viruses that infect humans and some other primate species. They are small (20 nm) replication-defective, nonenveloped viruses and have linear single-stranded DNA (ssDNA) genome of approximately 4.8 kilobases (kb). Several features make AAV an attractive candidate for creating viral vectors for gene therapy and for the creation of isogenic human disease models. AAV-based vectors have emerged as the preferred vector system for neuro-logic gene therapy, with good safety record in clinical trials.

The term “plasmid” refers to a genetic structure in a cell that can replicate independently of the chromosomes, typically a small circular DNA strand. Plasmids are often used in the laboratory manipulation of genes. Plasmids represent the simplest form of vector for transport of DNA into the cell nucleus. The generally include a circular, double-stranded DNA molecule varying in size from <1000 to >200 000 bp. Compared with recombinant viruses, plasmids are simple to construct and easily propagated in large quantities. They also possess an excellent safety profile, with virtually no risk of oncogenesis (as genomic integration is very inefficient) and relatively little immunogenicity. Plasmids can be administered by, for example, infusion or injection and can be administered using techniques (e.g., carrier vehicles) to improve their update by targeted cells.

A carrier vehicle for a plasmid (pDNA) can reduce susceptibility to circulating nucleases and increase cellular uptake. They may also target plasmids to a specific tissue. Like carrier microbubbles, most vehicles are cationic. Their positive charge enables electrostatic complex formation with negatively charged pDNA. Complexes are prepared with a residual positive charge which enhances cellular uptake via electrostatic interaction with the negatively charged cell membrane. These carrier vehicles can substantially increase transfection; non-viral systems can be as effective as viruses at delivering DNA to cell nuclei.

The term “episome” or “plasmid” refers to a length of DNA that exists either in the cytoplasm or attached to the chromosome of a mammalian cell. They replicate in synchrony with the host chromosome and are thus perpetuated as long as the parent strain exists. The main disadvantage of integrating vector systems is their potential risk of causing insertional mutagenesis. Episomal vector systems have the potential to avoid these undesired side effects because they behave as separate extrachromosomal elements in the nucleus of a target cell.

The term “subject” refers to those who are susceptible to an ailment (e.g., a disease related to senescence) or who are suspected of having or diagnosed with the ailment. However, any subject to be treated with the therapeutic methods described herein is included without limitation.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

Many known and useful compounds and the like can be found in Remington's Pharmaceutical Sciences (13^{th} Ed), Mack Publishing Company, Easton, PA—a standard reference for various types of administration. As used herein, the term “formulation(s)” refers to a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### DETAILED DESCRIPTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims hereof.

De novo synthesis of human mitochondria requires the concerted translation of nuclear and mitochondrial genes. The mitochondrial proteome includes more than 1500 proteins of which only 13 genes are encoded in the mitochondrial DNA (mtDNA). However, mtDNA is particularly vulnerable to damage and accumulation of mutations. Its location adjacent to the oxidative phosphorylation machinery exposes the genetic material to higher risk of mutagenic events. The mtDNA is susceptible to mutations from reactive oxygen species generated via oxidative phosphorylation as well as inadequate DNA repair mechanisms in the organelle.

Quality and quantity of mtDNA has been linked to mitochondrial function. Inadequate function can cause mitochondrial diseases. Senescence is also linked to mitochondrial function. Age-related changes in mitochondrial metabolism can lead to, for example, a decreased number of mitochondria, declining rates of ATP synthesis, or reduced oxidative capacity. Other mitochondrial changes observed in aging include mtDNA copy number depletion and large deletions in mtDNA coding regions.

Allotopic expression is a promising therapeutic tool to genetically remedy deleterious mtDNA mutations through nuclear complementation of the affected genes. In allotopic expression, a mitochondrial gene is deliberately recoded and relocated into the nucleus and the encoded polypeptide is imported back into the mitochondrion.

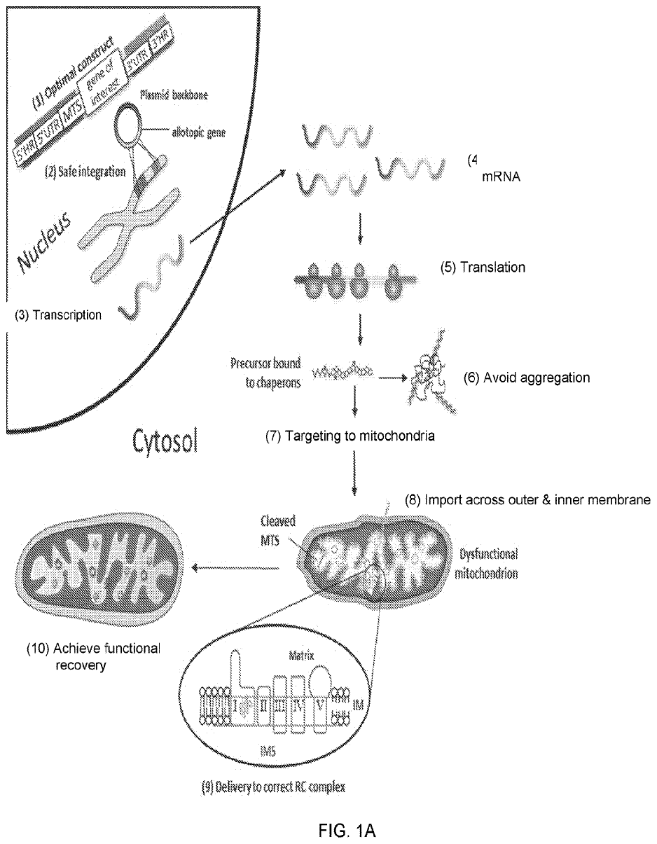

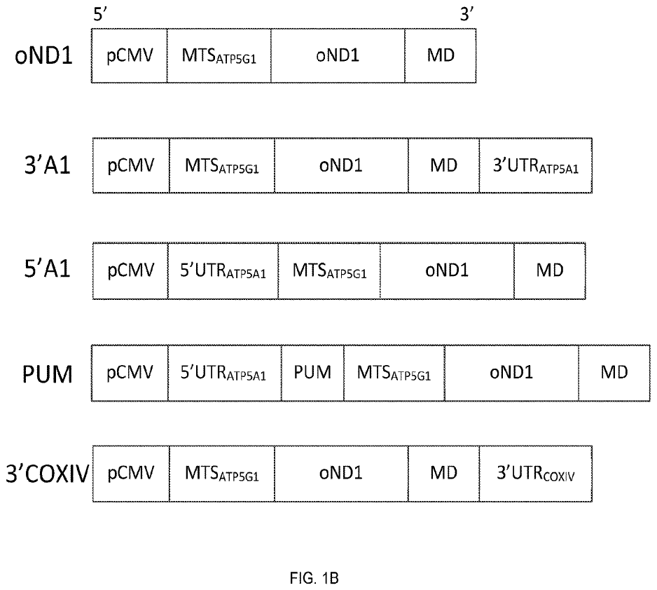

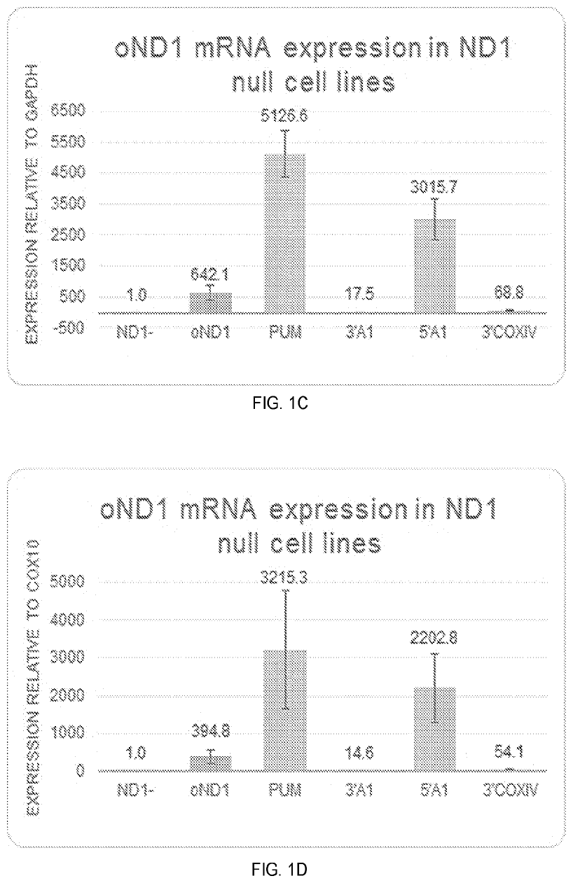

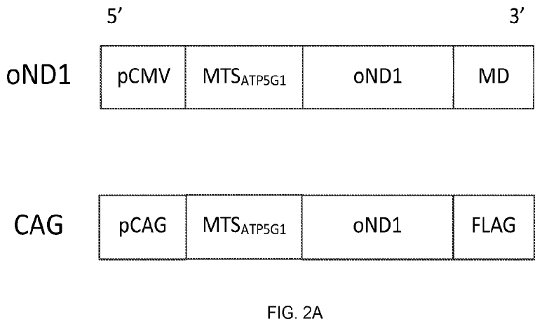

FIG. 1 is a schematic for allotopic expression that shows the various steps involved in the successful implementation of the strategy. The first step (1) is the design of the optimal DNA expression construct. As described herein, a modified or wild-type version of the mitochondrial gene can be used in the construct. Next, the plasmid is integrated into an allotopic gene (2). This step is followed by transcription (3). Next, sufficient amounts of the mRNA must be exported into the cytosol (4) and translated into a peptide (5). As described herein, efforts must be taken to avoid aggregation (6). The peptide is targeted to mitochondria (7) and imported across the outer and inner membrane of the mitochondria (8). Thereafter, the peptide is delivered to correct a component of the respiratory chain (RC) complex (8). If these steps are successful, the peptide is delivered to correct a deficiency in a mitochondrial deficiency (9) and functional recovery can be achieved (10).

Conventional methods of allotopic expression have been unsuccessful for numerous reasons. The present invention provides methods that overcome hurdles of past efforts of allotopic expression.
In embodiments of the present invention, the methods described herein address the differences in codon utilization preferences between genomes.
In embodiments, translational slowdown or ribosome stalling is induced during the synthesis of allotopically expressed (AE) proteins. This is especially relevant in conjunction with codon optimized constructs.
In embodiments, the influence of mitochondria import direction on the final, functional assembly of an AE subunit is considered. Topology changes that can result from post-translational import vs. matrix synthesis are also addressed.
In embodiments, specific mitochondrial import machinery or those components utilized by multi-pass inner membrane proteins are targeted.
In embodiments, the systematic reduction in the hydrophobicity of specific domains in the allotopic protein is addressed to improve import while preserving function.
In embodiments, feedback control of AE gene expression is also addressed.
In embodiments, a combination of synergistic elements is addressed to improve allotopic expression and targeting to mitochondria.
In embodiments, the methods described herein account for expression of several genes at the same time.

### Codon Optimization

Codon optimization refers to experimental approaches designed to improve the codon composition of a recombinant gene based on various criteria without altering the amino acid sequence. This is possible because most amino acids are encoded by more than one codon. The genetic code of mitochondria furthermore often differs from the standard genetic code. Mitochondrial genomes have a relatively conserved gene content and small size. Moreover, the relative use of particular codons for each amino acid is vastly different between the two genomes. This is a facet that has not been utilized in conventional methods of allotopic expression.

Codon optimization of transgenes can lead to an increased transcription (mRNA levels) by synchronizing transgene sequences to the codon preferences of the nuclear genome. Because the mitochondrial genome uses codons differently than the nuclear genome, all allotopically expressed genes require several changes to maintain the amino acid sequence of the encoded protein. The codon-optimized transgenes described herein results in greater transcription of mtDNA genes.

### N-Terminal Export

The import mechanisms by which soluble proteins are translocated across the membranes of peroxisomes, mitochondria, chloroplasts or of the ER are remarkably different. However, the targeting signals for mitochondria, chloroplasts, or the ER appear structurally similar, because they all involve an α-helical domain in proximity to the N-terminus. The targeting signals for mitochondria, chloroplasts, and the ER are encoded within N-terminal sequences with different denominations (presequence, transit sequence, and signal peptide).

The 13 mitochondrially-encoded proteins are all hydrophobic subunits of electron transport chain (ETC) enzyme complexes, and are necessary for oxidative phosphorylation (OXPHOS), which occurs across the mitochondrial inner membrane (MIM). When mitochondrial genes are normally expressed, their transcripts are translated within the mitochondrial matrix, the innermost mitochondrial compartment, and are subsequently inserted into the inner membrane. Each of these subunits contain one or more transmembrane domains spanning the MIM region, and adopt a final topology characterized by the position of the N- and C-termini of the protein with respect to the inter membrane space (IMS) and the matrix. Because mtDNA-encoded subunits are translated from within the matrix, proteins with their N-termini facing the IMS may have that end “exported” across the inner membrane. When these subunits are allotopically expressed, however, they are introduced to the mitochondria from the opposite face of the inner membrane, with the N-terminus feeding first into the matrix.

Previous studies have identified a six-residue sequence in the nuclear-encoded inner membrane protein OXA1L which is responsible for translocating (exporting) its N-terminus across the MIM following import, resulting in a final N-out (IMS-facing) topology. Therefore, for the allotopic expression of OXPHOS subunits which should adopt a final topology with the N-terminus facing the IMS, Applicants have included this six-residue “N-terminal export signal” in the construct, to stimulate translocation of the N-terminus back towards the IMS following initial import to the matrix.

### Transmembrane Architecture

Mitochondria are separated from the cytoplasm by the outer and inner mitochondrial membrane. The outer membrane is porous and freely traversed by ions and small, uncharged molecules through pore-forming membrane proteins (i.e., porins), such as the voltage-dependent anion channel (VDAC). Any larger molecules, especially proteins, must be imported by special translocases. Because of its porosity, there is no membrane potential across the outer membrane. By contrast, the inner membrane is a tight diffusion barrier to all ions and molecules. These can only get across with the aid of specific membrane transport proteins, each of which is selective for a particular ion or molecule. As a result of its ion selectivity, an electrochemical membrane potential of about 180 mV builds up across the inner mitochondrial membrane. The inner membrane is where oxidative phosphorylation takes place in a suite of membrane protein complexes that create the electrochemical gradient across the inner membrane or use it for ATP synthesis.

Many multi-pass inner membrane proteins are believed to have helices which translocate to the matrix as helix pairs, stabilizing one another. Paired translocation across the membrane/lateral insertion changes the in-out orientation of subsequent transmembrane regions.

Alternatively to (or in conjunction with) an N-terminal export signal, the addition of “inert” (non-interfering) transmembrane domain regions at either the amino or carboxy terminus of the AE protein can be included to restore the natural orientation of transmembrane helices with respect to the mitochondrial inner membrane. Loop regions connecting extra helices to the AE protein can include sites for cleavage by specific local proteases. Different cleavage signatures (e.g., for MMP vs OCT1 vs IMP vs IMS proteases) can be used depending on where the loop is located.

The transmembrane domain can also be used to reduce hydrophobicity. For example, specific sequences in the transmembrane domain can code for hydrophilic amino acids.

### Localized Charges

Studies have demonstrated that surface charge can determine protein localization. The surface charge can affect electrostatic interactions between anionic lipids and cationic amino acids which work in combination with other processes to direct protein localization. In embodiments, the surface charge of the AE protein is modulated or reduced.

For example, for ND1, net-negative charge of a reporter tag may also help facilitate association of the carboxy terminus of ND1 with the localized positive charges on the intermembrane space side of the inner membrane, preventing complete translocation and maintaining that terminus on the correct side of the MIM. Similarly, for A6, localized charges near the N-terminus can aid in stimulating interaction with the MIM-IMS interface to allow for proper topology (or by avoiding affecting the cryptic, endogenous signals seemingly present in the c-terminal region).

### Promoter Choice

In embodiments, cellular state is considered in regard to the promoter. Long-term adaptive expression of AE transgenes is often in response to cellular state (e.g., energy status). Inclusion of transcription factor binding sites can improve transcriptional regulation in response to cellular state (e.g., Pgc1a/err1a, HIF, NRF1/2 or other effectors). Also included are regulatory elements permitting tissue and condition-specific gene expression levels. Pcg1a/err1a target genes, for example, are highly expressed in tissues with high oxygen demand. Metabolic feedback loop/gene circuit can also be considered.

pCMV is recognized as a strong promoter and is often used in allotopic studies. However, because of strong constitutive expression, pCMV can lead to aggregation and thus proteotoxicity. In embodiments, the pCMV promoter is replaced with a pCAG promoter to influence an endogenous chimeric promoter. As described herein, pCAG does not cause the same degree of overexpression of downstream genes as pCMV.

There are additional benefits to using an alternative promoter such as pCAG. The existing pCMV constructs can have translation initiation sites within the promoter, which can generate an incorrect protein product if recognized by the cell. The pCAG promoter also has an upstream ORF, however an observed upstream stop site would prevent the formation of an aberrant promoter-fusion protein. uSTOPs can be included to correct translation initiation. In embodiments, a chimeric promoter has CMV enhancer sequence, however otherwise derived from chicken beta-globulin, and is known to have a better stable expression profile in safe harbor nuclear expression regions in mice compared to CMV, which can be silenced over time.

Other promoters that can be used include, for example, PGK, PGC1alpha, ER1alpha, or common tissue-specific promoters such as alphaMyosin, creatinine kinase, or hybrid promoters such as C5-12, MHCK7. Still others include AAV serotypes 1-9, AAV2/6 or AAV2/8.

Though there is no evidence of our CMV constructs being silenced (high mRNA levels are sustained), the expression from this promoter is predicted to be lower overall, which could be beneficial if constitutive, high expression levels (functional or not) are causing undue cellular proteostatic (or other) stress.

### Upstream Architecture and 5′ UTR

The 5′ UTR regions from specific classes of mitochondrial proteins have been appended upstream of the expression construct. In addition to general transfactor recognition, 5′ UTR regions may form stable structures which can improve mRNA quality or may facilitate transcript localization for translation near the mitochondrial surface.

Different UTR regions can improve mRNA/nascent chain targeting. Because CMV is a constitutive promoter, it does not usually require transcription factor induction. However, under some instances of cellular stress (such as particular mitochondrial stress) certain transcriptional programs are up- or down-regulated. One of these pathways is responsive to NRF1 (nuclear respiratory factor 1) which functions like PGC1a to activate OXPHOS genes in response to metabolic conditions and aerobic demand. Further, it is involved in the antioxidant response. Many mitochondrial genes are upregulated in response to NRF-1, including ATP5A1. A putative binding site is identified. ATP5A1 was chosen arbitrarily among NEMGs (nuclear-encoded mitochondrial genes). The 5′ UTR may also contain a Y-box promoter binding motif. Some examples include:
YYI binding
   ABCBA
   ATP5A1
NRF1/NRF2
   ATP5A1
uSTOP
   MPCP
   ABCBA
      An upstream in-frame stop codon near the translation start site can aid in translational initiation rates and prevent upstream starts from interfering with downstream translation.

In embodiments, a 5′ UTR of nuclear encoded mitochondrial protein (NUMP) is used. Examples include sequences of complex I, complex II, complex IV, complex V, inner membrane proteins or matrix proteins.

### 3′ UTR (COXIV)

COX4 3′UTR was similarly selected (somewhat arbitrary of NEMGs with various RBP binding sites). Contains a site for binding of YBX1, an RBP identified in stabilizing mRNA and helps regulate transcription by modulating interaction between EIFs and mRNA.

### 3′ UTR (ATP5A1)

The 3′ UTR of ATP5A1 is also being studied, as it contains both a PUM (pumilio) binding site, as well as a sequence similar to the potential CLUH sequence found to be enriched in mitochondrial proteins. CLUH was identified in drosophila as a protein which is implicated in the transport of OXPHOS mRNAs to the mitochondrial surface. In yeast, some PUM proteins assist in the local enrichment of OXPHOS transcripts at the OMM. Pum proteins influence translation dynamics by ribosome stalling, binding at the 3′ end of many mRNAs and forming secondary structures which inhibit (or delay) translation. PUM (pufp3 in yeast) binds the mRNAs of many mitochondria-targeted proteins, and is believed to be involved in both nutrient condition-dependent translation (when there is demand for OXPHOS) and in facilitating localized translation at the OMM. This UTR also forms a highly stable secondary structure, which has been implicated in general mRNA stability. It is proposed, in some instances, to also play a role in RBP recognition and binding.

In embodiments, a 3′ UTR of nuclear encoded mitochondrial protein (NUMP) is used. Examples include sequences of complex I, complex II, complex IV, complex V, inner membrane proteins or matrix proteins.

### Mitochondrial Targeting/MTS Selection

A target peptide is a short (3-70 amino acids long) peptide chain that directs the transport of a protein to a specific region in the cell (e.g., the mitochondria). The mitochondrial targeting signal (“MTS”) is a 10-70 amino acid long peptide that directs a newly synthesized protein to the mitochondria. It is predominantly found at the N-terminus end and includes an alternating pattern of hydrophobic and positively charged amino acids to form an amphipathic helix. Mitochondrial targeting signals can contain additional signals that subsequently target the protein to different regions of the mitochondria, such as the mitochondrial matrix or inner membrane.

Previous efforts toward targeting precursor proteins to the mitochondria have utilized either ATP5G1mts or COX VIII mts. Embodiments include an MTS sequence that overcomes limitations of MTS sequences used in conventional methods of allotopic expression. The MTS sequences described herein can:
Direct AE proteins to specific import machinery for nuclear mitochondrial proteins (NuMPs),
Differentiate targeting of the AE protein to the import machinery utilized by multipass, inner-membrane NUMPs, and
Facilitate recognition by outer membrane receptors as having a destination embedded in the MIM.
Provide complex specificity

Additionally, by utilizing MTSs from protein classes known to interact with specific chaperones, applicants take advantage of additional signals they may encode, such as those for chaperone recognition/recruitment (e.g., Hsp90), and outer membrane receptor recognition, as well as native context for codon composition, which may play a role in modulating translation dynamics (which, in turn, influence folding/misfolding, chaperone recognition, targeting, etc.).

### OXA1L

Endogenous context for HEX signal following OXA1L MTS. OXA1L is an inner-membrane insertase conserved from bacteria and utilizes the hexapeptide export signal in adopting its final topology. Because of the protein's structural similarity to many OXPHOS proteins embedded in the MIM, this MTS may encode additional information which aid in its targeting and insertion into the inner membrane.

Other embodiments could include sequences derived from other non-carrier, multipass, nuclear-encoded mitochondrial inner membrane proteins, such as: TMM70, TMM65, TMM11, TMM177, TI17B, T123B, TI17A, T126A, SURF1, SPNS1, SPG7, SFXN4, SFXN1, PARL, OMA1, NNTM, NDUAB, NCLX, MFTEP1, MRS2, MVP17, MIC27, MCUR1, MCUB, MCU, M17L2, IFI6, HIG2A, HIG1A, GHITM, DMAC1, DHSD, CRLS1, COX20*, COX18*, COQ2, CDS2, C560, AGK, AFG32*COX-derived sequences found in several existing patents.

### MTS (MPCP)

MPCP, a mitochondrial carrier family protein, is unique in that it has a cleaved N-terminal MTS, typically absent in the carrier proteins, which are imported via the TIM22 pathway rather than TIM23 pathway. The structure and biochemical properties of ND1 are similar to carrier family proteins, and thus cooperative import via the TIM23 and TIM22 pathways may be achieved through inclusion of this targeting region. (see: e.g., V Zara, F Palmieri, K Mahlke, N Pfanner, “The cleavable presequence is not essential for import and assembly of the phosphate carrier of mammalian mitochondria but enhances the specificity and efficiency of import,” Journal of Biological Chemistry, Volume 267, Issue 17, 1992).

Metabolite/carrier family proteins (which constitute the bulk of multipass MIM proteins) are specifically targeted to TOM70 on the outer membrane prior to being passed to TOM20/22 for import. TOM70 substrates go on to be inserted in the MIM via TIM22.

TOM70 is also a known interaction partner of Hsp90, a ubiquitous chaperone involved in facilitating and correcting protein folding. This suggests that Hsp90 specifically (unlike the even more ubiquitous Hsp70) may recognize and bind to sequences in nascent carrier proteins and aid in their delivery to MOM receptors in an import-competent state. Because HSPs bind promiscuously, no specific signal or motif is identified that can be appended to its constructs; thus, sequences instead derived from its client proteins are candidates for recognition. In this case, the presequence of MPCP may have dual benefit if it is both recognized as a misfolding-prone, mitochondria-destined client by Hsp90 chaperones, and if it further can direct inner membrane assembly via TIM22.

Furthermore, it has been shown that OXA1L, the insertase responsible for the (co-translational) inner membrane insertion and assembly of most mitochondrially-encoded OXPHOS subunits, is also required for the assembly of other multipass inner membrane proteins, including many of the metabolite carrier family (as well as many of the TIMs, SDH subunits, and ABC transporters). Thus, the presequence of MPCP may constitute a signal which can be recognized by the import and assembly machinery as being destined for the MIM, and potentially assist in targeting allotopically-expressed ND1 and other multi-pass subunits to the machinery responsible for correct inner membrane organization and final topology.

Other embodiments could include sequences derived from any of the mitochondrial carrier family proteins (gene names SLC25A) which include: UCP5, UCP4, UCP3, UCP2, UCP1, TXTP, TPC, SCMC3/2/1, SAMC, S2553, S2552, S2551, S2548, S2547, S2545, S2544, S2543, S2542, S25441, S2540, S2539, S2538, S2536, S2535, S2534, S2533, ORNT2*, ORNT1*, ODC, MTCH2, MTCH1, MPC2, MPC1, MFTC, MFRN2, MFRN1, MCATL, MCAT, M2OM, KMCP1, GHC1, GDC, DIC, CMC2 and ADT4. *ORNT1/2 are ornithine transporters, different from the OTC enzyme which is a therapeutic target for several patents.

### MTS (ABCBA)

ABCBA is a mitochondrial ABC transporter of the inner membrane and is also unique in that it possesses an N-terminal MTS, uncharacteristic of this family. It is also demonstrated that the long MTS region and subsequent first transmembrane domain can facilitate correct folding and topology of attached inner membrane proteins, but not for matrix proteins. For this reason we will test the ability of the MTS alone and the MTS plus first TM region of the ABCBA protein to direct topology of allotopically expressed ND1. MTS region and TM region afterward also contain a relative abundance of slow-reading codons, specifically for proline and serine (rare), which may play a role in modulating translation kinetics and/or facilitating trans-factor identification. See, e.g., Ethan R. Graf, XueZhao Zhang, Shan-Xue Jin, Michael W. Linhoff, Ann Marie Craig, Neurexins Induce Differentiation of GABA and Glutamate Postsynaptic Specializations via Neuroligins, Cell, Volume 119, Issue 7, 2004, pp. 1013-1026, and Mitsunori Miyazaki and Karyn A. Esser, “Cellular mechanisms regulating protein synthesis and skeletal muscle hypertrophy in animals,” Journal of Applied Physiology, Vol. 106, No. 4, 2005.

An allotopic protein (“AE protein”) can be paired with the MTS of a nuclear encoded mitochondrial protein (NUMP) from the same respiratory complex to facilitate complex integration. This includes other members of the mature respiratory complex, as well as assembly factors. In some cases, the precise location of the MTS is not known. An estimate may be made based on literature findings to incorporate an appropriate length of the protein N-terminus. In aspects, an allotopic protein is paired with an MTS of a protein identified in Table 1 (i.e., SEQ ID NO: 1-63).

TABLE 1
MTSs of Nuclear-Encoded Mitochondrial Proteins of Respiratory Complex I

| SEQ | | | Gene | | |
|---|---|---|---|---|---|
| ID: | AE Protein | NUMP | Synonyms | MTS Region | MTS sequence |
| 1 | ND1, ND2, ND3, ND4, ND4L, ND5 or | NDUV1_HUMAN | NDUFV1 | TRANSIT 1 . . . 20 | MLATRRLLGWSLP |
| | ND6 | | UQOR1 | | ARVSVRF |
| 2 | ND1, ND2, ND3, ND4, ND4L, ND5 or | NDUS1_HUMAN | NDUFS1 | TRANSIT 1 . . . 23 | MLRIPVRKALVGLS |
| | ND6 | | | | KSPKGCVRT |
| 3 | ND1, ND2, ND3, ND4, ND4L, ND5 or | CIA30_HUMAN | NDUFAF1 | TRANSIT 1 . . . 24 | MALVHKLLRGTYFL |
| | ND6 | | CIA30 | | RKFSKPTSAL |
| | | | CGI-65 | | |
| 4 | ND1, ND2, ND3, ND4, ND4L, ND5 or | NDUC1_HUMAN | NDUFC1 | TRANSIT 1 . . . 27 | MAPSALLRPLSRLL |
| | ND6 | | | | APARLPSGPSVRS |
| 5 | ND1, ND2, ND3, ND4, ND4L, ND5 or | NDUS6_HUMAN | NDUFS6 | TRANSIT 1 . . . 28 | MAAAMTFCRLLNR |
| | ND6 | | | | CGEAARSLPLGAR |
| | | | | | CF |
| 6 | ND1, ND2, ND3, ND4, ND4L, ND5 or | NDUB8_HUMAN | NDUFB8 | TRANSIT 1 . . . 28 | MAVARAGVLGVQ |
| | ND6 | | | | WLQRASRNVMPL |
| | | | | | GART |
| 7 | ND1, ND2, ND3, ND4, ND4L, ND5 or | NDUBB_HUMAN | NDUFB11 | TRANSIT 1 . . . 29 | MAAGLFGLSARRL |
| | ND6 | | UNQ111/ | | LAAAATRGLPAAR |
| | | | PRO1064 | | VRW |
| 8 | ND1, ND2, ND3, ND4, ND4L, ND5 or | NDUV2_HUMAN | NDUFV2 | TRANSIT 1 . . . 32 | MFFSAALRARAAG |
| | ND6 | | | | LTAHWGRHVRNLH |
| | | | | | KTVMQN |
| 9 | ND1, ND2, ND3, ND4, ND4L, ND5 or | NDUS2_HUMAN | NDUFS2 | TRANSIT 1 . . . 33 | MAALRALCGFRGV |
| | ND6 | | | | AAQVLRPGAGVRL |
| | | | | | PIQPSRG |
| 10 | ND1, ND2, ND3, ND4, ND4L, ND5 or | NDUB2_HUMAN | NDUFB2 | TRANSIT 1 . . . 33 | MSALTRLASFARV |
| | ND6 | | | | GGRLFRSGCARTA |
| | | | | | GDGGVRH |
| 11 | ND1, ND2, ND3, ND4, ND4L, ND5 or | NDUV3_HUMAN | NDUFV3 | TRANSIT 1 . . . 34 | MAAPCLLRQGRAG |
| | ND6 | | | | ALKTMLQEAQVFR |
| | | | | | GLASTVSL |
| 12 | ND1, ND2, ND3, ND4, ND4L, ND5 or | NDUS8_HUMAN | NDUFS8 | TRANSIT 1 . . . 34 | MRCLTTPMLLRAL |
| | ND6 | | | | AQAARAGPPGGR |
| | | | | | SLHSSAVAA |
| 13 | ND1, ND2, ND3, ND4, ND4L, ND5 or | NDUA9_HUMAN | NDUFA9 | TRANSIT 1 . . . 35 | MAAAAQSRVVRVL |
| | ND6 | | NDUFS2L | | SMSRSAITAIATSV |
| | | | | | CHGPPCRQ |
| 14 | ND1, ND2, ND3, ND4, ND4L, ND5 or | NDUAA_HUMAN | NDUFA10 | TRANSIT 1 . . . 35 | MALRLLKLAATSAS |
| | ND6 | | | | ARVVAAGAQRVRG |
| | | | | | IHSSVQCK |
| 15 | ND1, ND2, ND3, ND4, ND4L, ND5 or | NDUS3_HUMAN | NDUFS3 | TRANSIT 1 . . . 36 | MAAAAVARLWWR |
| | ND6 | | | | GILGASALTRGTGR |
| | | | | | PSVLLLPVRR |
| 16 | ND1, ND2, ND3, ND4, ND4L, ND5 or | NDUS7_HUMAN | NDUFS7 | TRANSIT 1 . . . 38 | MAVLSAPGLRGFRI |
| | ND6 | | | | LGLRSSVGPAVQA |
| | | | | | RGVHQSVATDG |
| 17 | ND1, ND2, ND3, ND4, ND4L, ND5 or | NDUS4_HUMAN | NDUFS4 | TRANSIT 1 . . . 42 | MAAVSMSVVLRQT |
| | ND6 | | | | LWRRRAVAVAALS |
| | | | | | VSRVPTRSLRTST |
| | | | | | WRL |
| 18 | ND1, ND2, ND3, ND4, ND4L, ND5 or | NDUB5_HUMAN | NDUFB5 | TRANSIT 1 . . . 46 | MAAMSLLRRVSVT |
| | ND6 | | | | AVAALSGRPLGTR |
| | | | | | LGFGGFLTRGFPK |
| | | | | | AAAPVRH |
| 19 | ND1, ND2, ND3, ND4, ND4L, ND5 or | ACPM_HUMAN | NDUFAB1 | TRANSIT 1 . . . 68 | MASRVLSAYVSRL |
| | ND6 | | | | PAAFAPLPRVRML |
| | | | | | AVARPLSTALCSA |
| | | | | | GTQTRLGTLQPAL |
| | | | | | VLAQVPGRVTQLC |
| | | | | | RQY |
| 20 | ND1, ND2, ND3, ND4, ND4L, ND5 or | OXA1L_HUMAN | OXA1L | TRANSIT | MVTWLYRFLP |
| | ND6 | | | 1 . . . 131 | TSNMAAKLRS |
| | | | | | LLPPDLRLQF |
| | | | | | WLHARLQKCF |
| | | | | | LSRGCGSYCA |
| | | | | | GAKASPLPGK |
| | | | | | MAMGLMCGRR |
| | | | | | ELLRLLQSGR |
| | | | | | RVHSVAGPSQ |
| | | | | | WLGKPLTTRL |
| | | | | | LFPVAPCCCR |
| | | | | | PHYLFLAASG |
| | | | | | PRSLSTSAIS |
| 21 | ND1, ND2, ND3, ND4, ND4L, ND5 or | NDUF2_HUMAN | NDUFAF2 | TRANSIT 1 . . . ? | (unavailable) |
| | ND6 | | NDUFA12L | | |
| 22 | ND1, ND2, ND3, ND4, ND4L, ND5 or | TIM21_HUMAN | TIMM21 | TRANSIT 1 . . . 18 | MICTFLRAVQYTEK |
| | ND6 | | C18orf55 | | LHRS |
| | | | TIM21 | | |
| | | | HSPC154 | | |
| 23 | ND1, ND2, ND3, ND4, ND4L, ND5 or | NDUF5_HUMAN | NDUFAF5 | TRANSIT 1 . . . 36 | MLRPAGLWRLCRR |
| | ND6 | | C20orf7 | | PWAARVPAENLGR |
| | | | | | REVTSGVSPR |
| 24 | ND1, ND2, ND3, ND4, ND4L, ND5 or | ACAD9_HUMAN | ACAD9 | TRANSIT 1 . . . 37 | MSGCGLFLRTTAA |
| | ND6 | | | | ARACRGLVVSTAN |
| | | | | | RRLLRTSPPVR |
| 25 | ND1, ND2, ND3, ND4, ND4L, ND5 or | NUBPL_HUMAN | NUBPL | TRANSIT 1 . . . 38 | MGIWQRLLLFGGV |
| | ND6 | | C14orf127 | | SLRAGGGATAPLG |
| | | | | | GSRAMVCGRQLS |
| 26 | ND1, ND2, ND3, ND4, ND4L, ND5 or | NDUF6_HUMAN | NDUFAF6 | TRANSIT 1 . . . 44 | MAASAHGSVWGP |
| | ND6 | | C8orf38 | | LRLGIPGLCCRRPP |
| | | | | | LGLYARMRRLPGP |
| | | | | | EVSGR |
| 27 | ND1, ND2, ND3, ND4, ND4L, ND5 or | NDUF7_HUMAN | NDUFAF7 | TRANSIT 1 . . . 46 | MSVLLRSGLGPLC |
| | ND6 | | C2orf56 | | AVARAAIPFIWRGK |
| | | | PRO1853 | | YFSSGNEPAENPV |
| | | | | | TPMLRH |
| 28 | ND1, ND2, ND3, ND4, ND4L, ND5 or | ECSIT_HUMAN | ECSIT | TRANSIT 1 . . . 48 | MSWVQATLLARGL |
| | ND6 | | | | CRAWGGTCGAAL |
| | | | | | TGTSISQVPRRLPR |
| | | | | | GLHCSAAAH |
| 29 | ND1, ND2, ND3, ND4, ND4L, ND5 or | AIFM1_HUMAN | AIFM1 AIF | TRANSIT 1 . . . 54 | MFRCGGLAAGALK |
| | ND6 | | PDCD8 | | QKLVPLVRTVCVR |
| | | | | | SPRQRNRLPGNLF |
| | | | | | QRWHVPLELQMT |
| | | | | | RQM |

TABLE 2
MTSs of Nuclear-Encoded Proteins of Complex V

| SEQ | | | | | |
|---|---|---|---|---|---|
| ID: | AE Protein | NUMP | Gene Synonyms | MTS Region | MTS sequence |
| 30 | ATP6, ATP8 | ATPD_HUMAN | ATP5F1D ATP5D | TRANSIT 1 . . . 22 | MLPAALLRRPGLGRLVRHARAY |
| 31 | ATP6, ATP8 | ATPO_HUMAN | ATP5PO ATP50 ATPO | TRANSIT 1 . . . 23 | MAAPAVSGLSRQVRCFSTSVVRP |
| 32 | ATP6, ATP8 | ATPG_HUMAN | ATP5F1C ATP5C | TRANSIT 1 . . . 25 | MFSRAGVAGLSAWTLQPQWIQVRN |
| | | | ATP5C1 ATP5CL1 | | M |
| 33 | ATP6, ATP8 | ATP5J_HUMAN | ATP5PF ATP5A ATP5J | TRANSIT 1 . . . 32 | MILQRLFRFSSVIRSAVSVHLRRNIG |
| | | | ATPM | | VTAVAF |
| 34 | ATP6, ATP8 | ATP5S_HUMAN | DMAC2L ATP5S ATPW | TRANSIT 1 . . . 40 | MCCAVSEQRLTCADQMMPFGKISQ |
| | | | | | QLCGVKKLPWSCDSRY |
| 35 | ATP6, ATP8 | AT5F1_HUMAN | ATP5PB ATP5F1 | TRANSIT 1 . . . 42 | MLSRVVLSAAATAAPSLKNAAFLGP |
| | | | | | GVLQATRTFHTGQPHLV |
| 36 | ATP6, ATP8 | ATPA_HUMAN | ATP5F1A ATP5A | TRANSIT 1 . . . 43 | MLSVRVAAAVVRALPRRAGLVSRN |
| | | | ATP5A1 ATP5AL2 | | ALGSSFIAARNFHASNTHL |
| | | | ATPM | | |
| 37 | ATP6, ATP8 | ATPB_HUMAN | ATP5F1B ATP5B | TRANSIT 1 . . . 47 | MLGFVGRVAAAPASGALRRLTPSA |
| | | | ATPMB ATPSB | | SLPPAQLLLRAAPTAVHPVRDYA |
| 38 | ATP6, ATP8 | AT5G1_HUMAN | ATP5MC1 ATP5G1 | TRANSIT 1 . . . 61 | MQTAGALFISPALIRCCTRGLIRPVS |
| | | | | | ASFLNSPVNSSKQPSYSNFPLQVAR |
| | | | | | REFQTSVVSR |
| 39 | ATP6, ATP8 | AT5G2_HUMAN | ATP5MC2 ATP5G2 | TRANSIT 1 . . . 66 | MFACSKFVSTPSLVKSTSQLLSRPL |
| | | | PSEC0033 | | SAVVLKRPEILTDESLSSLAVSCPLT |
| | | | | | SLVSSRSFQTSAISR |
| 40 | ATP6, ATP8 | AT5G3_HUMAN | ATP5MC3 ATP5G3 | TRANSIT 1 . . . 67 | MFACAKLACTPSLIRAGSRVAYRPIS |
| | | | | | ASVLSRPEASRTGEGSTVENGAQN |
| | | | | | GVSQLIQREFQTSAISR |
| 41 | ATP6, ATP8 | ATPD_HUMAN | ATP5F1D ATP5D | TRANSIT 1 . . . 22 | MLPAALLRRPGLGRLVRHARAY |
| 42 | ATP6, ATP8 | ATPO_HUMAN | ATP5PO ATP50 ATPO | TRANSIT 1 . . . 23 | MAAPAVSGLSRQVRCFSTSVVRP |
| 43 | ATP6, ATP8 | ATPG_HUMAN | ATP5F1C ATP5C | TRANSIT 1 . . . 25 | MFSRAGVAGLSAWTLQPQWIQVRN |
| | | | ATP5C1 ATP5CL1 | | M |
| 44 | ATP6, ATP8 | TMM70_HUMAN | TMEM70 | TRANSIT 1 . . . 81? | MLFLALGSPWAVELPLCGRRTALCA |
| | | | | | AAALRGPRASVSRASSSSG |
| | | | | | PSGPVAGWSTGPSGAARLLRRPGR |
| | | | | | AQIPVYWEGYVRF |
| 45 | ATP6, ATP8 | TM242_HUMAN | TMEM242 C6orf35 BM- | TRANSIT 1 . . . 22 | MLPAALLRRPGLGRLVRHARAY |
| | | | 033 HSPC287 | | |
| 46 | ATP6, ATP8 | ATP23_HUMAN | ATP23 KUB3 | TRANSIT 1 . . . 40 | MWRSCLRLRDGGRRLLNRPAGGP |
| | | | XRCC6BP1 | | SASMSPGPTIPSPARAY |
| 47 | ATP6, ATP8 | FMC1_HUMAN | FMC1 C7orf55 | TRANSIT 1 . . . 57 | MAAVVVAAAGGAGPAVLQVAGLYR |
| | | | HSPC268 | | GLCAVRSRALGLGLVSPAQLRVFPV |
| | | | | | RPGSGRPE |
| 48 | ATP6, ATP8 | ATPF1_HUMAN | ATPAF1 ATP11 | TRANSIT 1 . . . 81 | MLFLALGSPWAVELPLCGRRTALCA |
| | | | | | AAALRGPRASVSRASSSSGPSGPV |
| | | | | | AGWSTGPSGAARLLRRPGRAQIPV |
| | | | | | YWEGYVRF |

TABLE 3
MTSs of Nuclear-Encoded Proteins of Complex IV

| | AE | | | | |
|---|---|---|---|---|---|
| SEQ | Pro- | | | | |
| ID: | tein | NUMP | Gene Synonyms | MTS Region | MTS sequence |
| 49 | CYB | PT100_HUMAN | PET100 C19orf79 | TRANSIT | No cleavable |
| | | | | | pre-sequence |
| 50 | CYB | COX18_HUMAN | COX18 OXA1L2 | TRANSIT | MLCRLGGRWL |
| | | | | 1 . . . 20? | RPLPALQLWA |
| 51 | CYB | FAKD3_HUMAN | FASTKD3 | TRANSIT | (unknown) |
| | | | | 1 . . . ? | |
| 52 | CYB | TIM21_HUMAN | TIMM21 C18orf55 TIM21 | TRANSIT | MICTFLRAVQYTEKLHRS |
| | | | HSPC154 | 1 . . . 18 | |
| 53 | CYB | PT117_HUMAN | PET117 UNQ607/PRO1194 | TRANSIT | MSRSSKVVLGLSVLLTAATVA |
| | | | | 1 . . . 22 | G |
| 54 | CYB | COA8_HUMAN | COA8 APOP1 APOPT1 | TRANSIT | MLPCAAGARGRGAMVVLRAG |
| | | | C14orf153 | 1 . . . 39 | KKTFLPPLCRAFACRGCQL |
| 55 | CYB | SCO2_HUMAN | SCO2 | TRANSIT | MLLLTRSPTAWHRLSQLKPRV |
| | | | | 1 . . . 41 | LPGTLGGQALHLRSWLLSRQ |
| 56 | CYB | SCO1_HUMAN | SCO1 SCOD1 | TRANSIT | MAMLVLVPGRVMRPLGGQLW |
| | | | | 1 . . . 67 | RFLPRGLEFWGPAEGTARVLL |
| | | | | | RQFCARQAEAWRASGRPGY |
| | | | | | CLGTRPL |

TABLE 4
MTSs of Nuclear-Encoded Proteins of Complex III

| SEQ ID: | AE Protein | NUMP | Gene Synonyms | MTS Region | MTS sequence |
|---|---|---|---|---|---|
| 57 | COX1, COX2, COX3 | UQCC2_HUMAN | UQCC2 C6orf125 | TRANSIT | MAASRYRRFLKLC |
| | | | MNF1 | 1 . . . 13 | |
| 58 | COX1, COX2, COX3 | QCR6_HUMAN | UQCRH | TRANSIT | MGLEDEQKMLTES |
| | | | | 1 . . . 13 | |
| 59 | COX1, COX2, COX3 | QCR6L_HUMAN | UQCRHL | TRANSIT | MGLEDEQKMLTES |
| | | | | 1 . . . 13 | |
| 60 | COX1, COX2, COX3 | QCR2_HUMAN | UQCRC2 | TRANSIT | MKLLTRAGSFSRFY |
| | | | | 1 . . . 14 | |
| 61 | COX1, COX2, COX3 | QCR1_HUMAN | UQCRC1 | TRANSIT | MAASVVCRAATAGAQ |
| | | | | 1 . . . 34 | VLLRARRSPALLRTPAL |
| | | | | | RS |
| 62 | COX1, COX2, COX3 | TTC19_HUMAN | TTC19 | TRANSIT | MFRLLSWSLGRGFLRA |
| | | | | 1 . . . 70 | AGRRCRGCSARLLPGL |
| | | | | | AGGPGPEVQVPPSRV |
| | | | | | APHGRGPGLLPLLAAL |
| | | | | | AWFSRPA |
| 63 | COX1, COX2, COX3 | CY1_HUMAN | CYC1 | TRANSIT | MAAAAASLRGVVLGP |
| | | | | 1 . . . 84 | RGAGLPGARARGLLCS |
| | | | | | ARPGQLPLRTPQAVAL |
| | | | | | SSKSGLSRGRKVMLSA |
| | | | | | LGMLAAGGAGLAMAL |
| | | | | | HSAVSA |

In aspects, MTS's from transmembrane domain (TM) containing nuclear-encoded proteins are appended to allotopically expressed proteins on the basis of structural and biochemical properties, such as length and hydrophobicity. All mitochondrially-encoded proteins are integral components of membrane complexes, and as such, contain hydrophobic TM domains. Allotopically expressed mitochondrial proteins may therefore be appended with regions of similar TM domain containing proteins, which localize to the mitochondrial inner membrane. This can include the MTS of a nuclear-encoded protein. Alternatively, many inner membrane proteins with hydrophobic TM domains lack a canonical N-terminal MTS, and instead are targeted via cryptic internal sequences. In such instances, a region corresponding to the N-terminus of the protein through the first TM domain, plus (e.g., 1-30 AAs) can instead be appended. Table 5 lists examples of MTS sequences of transmembrane domain-containing nuclear encoded proteins of the inner membrane. In aspects, an allotopic protein is paired with an MTS of a protein identified in Table 5 or Table 6 (i.e., SEQ ID NO: 64-280).

TABLE 5
MTS Sequences of Transmembrane Domain-Containing Nuclear Encoded
Proteins of the Inner Membrane

| SEQ | Uniprot | | Human | | | |
|---|---|---|---|---|---|---|
| ID: | Identifier | Entry Name | Gene | MTS | MTS Sequence | TMs |
| 64 | O75027 | ABCB7_HUMAN | ABCB7 | TRANSIT 1 . . . 22 | MALLAMHSWRWAAAAAAFEKR | 6 |
| | | | ABC7 | | R | |
| 65 | Q9NRK6 | ABCBA_HUMAN | ABCB10 | TRANSIT | MRGPPAWPLRLLEPPSPAEPG | 5 |
| | | | | 1 . . . 105 | RLLPVACVWAAASRVPGSLSP | |
| | | | | | FTGLRPARLWGAGPALLWGVG | |
| | | | | | AARRWRSGCRGGGPGASRGV | |
| | | | | | LGLARLLGLWARGPGSCRCGA | |
| | | | | | F | |
| 66 | Q9Y4W6 | AFG32_HUMAN | AFG3L2 | TRANSIT 1 . . . 38 | MAHRCLRLWGRGGCWPRGLQ | 2 |
| | | | | | QLLVPGGVGPGEQPCLRT | |
| 67 | P05496 | AT5G1_HUMAN | ATP5MC1 | TRANSIT 1 . . . 61 | MQTAGALFISPALIRCCTRGLIR | 2 |
| | | | ATP5G1 | | PVSASFLNSPVNSSKQPSYSNF | |
| | | | | | PLQVARREFQTSVVSR | |
| 68 | Q06055 | AT5G2_HUMAN | ATP5MC2 | TRANSIT 1 . . . 66 | MFACSKFVSTPSLVKSTSQLLS | 2 |
| | | | ATP5G2 | | RPLSAVVLKRPEILTDESLSSLA | |
| | | | PSEC0033 | | VSCPLTSLVSSRSFQTSAISR | |
| 69 | P48201 | AT5G3_HUMAN | ATP5MC3 | TRANSIT 1 . . . 67 | MFACAKLACTPSLIRAGSRVAY | 2 |
| | | | ATP5G3 | | RPISASVLSRPEASRTGEGSTV | |
| | | | | | FNGAQNGVSQLIQREFQTSAIS | |
| | | | | | R | |
| 70 | Q99643 | C560_HUMAN | SDHC | TRANSIT 1 . . . 29 | MAALLLRHVGRHCLRAHFSPQ | 3 |
| | | | CYB560 | | LCIRNAVP | |
| | | | SDH3 | | | |
| 71 | Q96H96 | COQ2_HUMAN | COQ2 | TRANSIT 1 . . . 34 | MLGSRAAGFARGLRALALAWL | 9 |
| | | | CL640 | | PGWRGRSFALARA | |
| 72 | Q12887 | COX10_HUMAN | COX10 | TRANSIT 1.28 | MAASPHTLSSRLLTGCVGGSV | 7 |
| | | | | | WYLERRT | |
| 73 | Q9Y6N1 | COX11_HUMAN | COX11 | TRANSIT 1 . . . 28 | MGGLWRPGWR | 1 |
| | | | | | CVPFCGWRWI HPGSPTRA | |
| 74 | Q8N8Q8 | COX18_HUMAN | COX18 | TRANSIT | MLCRLGGRWL RPLPALQLWA | 3 |
| | | | OXA1L2 | 1 . . . 20? | | |
| 75 | P13073 | COX41_HUMAN | COX411 | TRANSIT 1 . . . 22 | MLATRVFSLVGKRAISTSVCVR | 1 |
| | | | COX4 | | MFPLVKSALNRLQVRSIQQTMA | |
| 76 | P24311 | COX7B_HUMAN | COX7B | TRANSIT 1 . . . 24 | RQ | 1 |
| 77 | P15954 | COX7C_HUMAN | COX7C | TRANSIT 1 . . . 16 | MLGQSIRRFTTSVVRR | 1 |
| 78 | P10176 | COX8A_HUMAN | COX8A | TRANSIT 1 . . . 25 | MSVLTPLLLRGLTGSARRLPVP | 1 |
| | | | COX8 | | RAK | |
| | | | COX8L | | | |
| 79 | P12074 | CX6A1_HUMAN | COX6A1 | TRANSIT 1 . . . 24 | MAVVGVSSVSRLLGRSRPQLG | 1 |
| | | | COX6AL | | RPM | |
| 80 | P08574 | CY1_HUMAN | CYC1 | TRANSIT 1 . . . 84 | MAAAAASLRGVVLGPRGAGLP | 1 |
| | | | | | GARARGLLCSARPGQLPLRTP | |
| | | | | | QAVALSSKSGLSRGRKVMLSA | |
| | | | | | LGMLAAGGAGLAMALHSAVSA | |
| 81 | O14521 | DHSD_HUMAN | SDHD | TRANSIT 1 . . . 56 | MAVLWRLSAVCGALGGRALLL | 3 |
| | | | SDH4 | | RTPVVRPAHISAFLQDRPIPEW | |
| | | | | | CGVQHIHLSPSHH | |
| 82 | Q96LL9 | DJC30_HUMAN | DNAJC30 | TRANSIT 1 . . . 38 | MAAMRWRWWQRLLPWRLLQA | 1 |
| | | | WBSCR18 | | RGFPQNSAPSLGLGARTY | |
| 83 | Q9H419 | EMRE_HUMAN | SMDT1 | TRANSIT 1 . . . 52 | MASGAARWLVLAPVRSGALRS | 1 |
| | | | C22orf32 | | GPSLRKDGDVSAAWSGSGRSL | |
| | | | EMRE | | VPSRSVIVTR | |
| 84 | Q9H3K2 | GHITM_HUMAN | GHITM | TRANSIT 1 . . . 45 | MLAARLVCLRTLPSRVFHPAFT | 7 |
| | | | DERP2 | | KASPVVKNSITKNQWLLTPSRE | |
| | | | MICS1 | | Y | |
| | | | TMBIM5 | | | |
| | | | My021 | | | |
| | | | UNQ244/P | | | |
| | | | RO281 | | | |
| 85 | O95202 | LETM1_HUMAN | LETM1 | TRANSIT | MASILLRSCRGRAPARLPPPPR | 1 |
| | | | | 1 . . . 115 | YTVPRGSPGDPAHLSCASTLGL | |
| | | | | | RNCLNVPFGCCTPIHPVYTSSR | |
| | | | | | GDHLGCWALRPECLRIVSRAP | |
| | | | | | WTSTSVGFVAVGPQCLPVRG | |
| | | | | | WHSSRPVR | |
| 86 | Q2VYF4 | LETM2_HUMAN | LETM2 | TRANSIT 1 . . . 25 | MAFYSYNSVLAIARTRFPSHFV | 1 |
| | | | | | HPT | |
| 87 | Q8NE86 | MCU_HUMAN | MCU | TRANSIT 1 . . . 50 | MAAAAGRSLLLLLSSRGGGGG | 2 |
| | | | C10orf42 | | GAGGCGALTAGCFPGLGVSRH | |
| | | | CCDC109A | | RQQQHHRT | |
| 88 | Q9NWR8 | MCUB_HUMAN | MCUB | TRANSIT 1 . . . 35 | MLQRGLWPWRTRLLPTPGTW | 2 |
| | | | CCDC109B | | RPARPWPLPPPPQVL | |
| 89 | Q6UXV4 | MIC27_HUMAN | APOOL | TRANSIT 1 . . . 27 | MAAIRMGKLTTMPAGLIYASVS | 2 |
| | | | CXorf33 | | VHAAK | |
| | | | FAM121A | | | |
| | | | MIC27 | | | |
| | | | UNQ8193/ | | | |
| | | | PRO23204 | | | |
| 90 | Q16891 | MIC60_HUMAN | IMMT HMP | TRANSIT 1 . . . 33 | MLRACQLSGVTAAAQSCLCGK | 1 |
| | | | MIC60 | | FVLRPLRPCRRY | |
| | | | MINOS2 | | | |
| | | | PIG4 | | | |
| | | | PIG52 | | | |
| 91 | Q9BPX6 | MICU1_HUMAN | MICU1 | TRANSIT 1 . . . 33 | MFRLNSLSALAELAVGSRWYH | 1 |
| | | | CALC | | GGSQPIQIRRRL | |
| | | | CBARA1 | | | |
| 92 | Q96ER9 | MITOK_HUMAN | CCDC51 | TRANSIT 1 . . . 35 | MMGRSPGFAMQHIVGVPHVLV | 2 |
| | | | MITOK | | RRGLLGRDLFMTRT | |
| 93 | Q9NUT2 | MITOS_HUMAN | ABCB8 | TRANSIT 1 . . . 25 | MLVHLFRVGIRGGPFPGRLLPP | 3 |
| | | | MABC1 | | LRF | |
| | | | MITOSUR | | | |
| 94 | Q00325 | MPCP_HUMAN | SLC25A3 | TRANSIT 1 . . . 49 | MFSSVAHLARANPFNTPHLQLV | 6 |
| | | | PHC | | HDGLGDLRSSSPGPTGQPRRP | |
| | | | OK/SW- | | RNLAAA | |
| | | | cl.48 | | | |
| 95 | Q9HD23 | MRS2_HUMAN | MRS2 HPT | TRANSIT 1 . . . 49 | MECLRSLPCLLPRAMRLPRRTL | 2 |
| | | | MRS2L | | CALALDVTSVGPPVAACGRRA | |
| | | | | | NLIGRS | |
| 96 | Q6J4K2 | NCLX_HUMAN | SLC8B1 | TRANSIT 1 . . . 26 | MAGRRLNLRWALSVLCVLLMA | 13 |
| | | | NCKX6 | | ETVSG | |
| | | | NCLX | | | |
| | | | SLC24A6 | | | |
| 97 | O43674 | NDUB5_HUMAN | NDUFB5 | TRANSIT 1 . . . 46 | MAAMSLLRRVSVTAVAALSGR | 1 |
| | | | | | PLGTRLGFGGFLTRGFPKAAAP | |
| | | | | | VRH | |
| 98 | O95169 | NDUB8_HUMAN | NDUFB8 | TRANSIT 1 . . . 28 | MAVARAGVLGVQWLQRASRN | 1 |
| | | | | | VMPLGART | |
| 99 | Q9NX14 | NDUBB_HUMAN | NDUFB11 | TRANSIT 1 . . . 29 | MAAGLFGLSARRLLAAAATRGL | 1 |
| | | | UNQ111/P | | PAARVRW | |
| | | | RO1064 | | | |
| 100 | O43677 | NDUC1_HUMAN | NDUFC1 | TRANSIT 1 . . . 27 | MAPSALLRPLSRLLAPARLPSG | 1 |
| | | | | | PSVRS | |
| 101 | Q13423 | NNTM_HUMAN | NNT | TRANSIT 1 . . . 43 | MANLLKTVVTGCSCPLLSNLGS | 14 |
| | | | | | CKGLRVKKDFLRTFYTHQELW | |
| 102 | Q96E52 | OMA1_HUMAN | OMA1 | TRANSIT 1 . . . 13 | MSFICGLQSAARN | 1 |
| | | | MPRP1 | | | |
| 103 | O60313 | OPA1_HUMAN | OPA1 | TRANSIT 1 . . . 87 | MWRLRRAAVACEVCQSLVKHS | 1 |
| | | | KIAA0567 | | SGIKGSLPLQKLHLVSRSIYHSH | |
| | | | | | HPTLKLQRPQLRTSFQQFSSLT | |
| | | | | | NLPLRKLKFSPIKYGYQPRRN | |
| 104 | Q15070 | OXA1L_HUMAN | OXA1L | TRANSIT | MAMGLMCGRRELLRLLQSGRR | 5 |
| | | | | 1 . . . 71? | VHSVAGPSQWLGKPLTTRLLFP | |
| | | | | | AAPCCCRPHYLFLAASGPRSLS | |
| | | | | | TSAISF-A | |
| 105 | Q9H300 | PARL_HUMAN | PARL | TRANSIT 1 . . . 52 | MAWRGWAQRGWGCGQAWG | 7 |
| | | | PSARL | | ASVGGRSCEELTAVLTPPQLLG | |
| | | | PRO2207 | | RRFNFFIQQKCG | |
| 106 | Q9BXM7 | PINK1_HUMAN | PINK1 | TRANSIT 1 . . . 77 | MAVRQALGRGLQLGRALLLRF | 1 |
| | | | | | TGKPGRAYGLGRPGPAAGCVR | |
| | | | | | GERPGWAAGPGAEPRRVGLG | |
| | | | | | LPNRLRFFRQSVAGL | |
| 107 | Q9UG56 | PISD_HUMAN | PISD | TRANSIT 1 . . . 52 | MATSVGHRCLGLLHGVAPWRS | 1 |
| | | | | | SLHPCEITALSQSLQPLRKLPFR | |
| | | | | | AFRTDARK | |
| 108 | PODJ07 | PT100_HUMAN | PET100 | TRANSIT 1 . . . ? | (unknown) | 1 |
| | | | C19orf79 | | | |
| 109 | Q02127 | PYRD_HUMAN | DHODH | TRANSIT 1 . . . 10 | MAWRHLKKRA | 1 |
| 110 | O75880 | SCO1_HUMAN | SCO1 | TRANSIT 1 . . . 67 | MAMLVLVPGRVMRPLGGQLW | 1 |
| | | | SCOD1 | | RFLPRGLEFWGPAEGTARVLL | |
| | | | | | RQFCARQAEAWRASGRPGYC | |
| | | | | | LGTRPL | |
| 111 | O43819 | SCO2_HUMAN | SCO2 | TRANSIT 1 . . . 41 | MLLLTRSPTAWHRLSQLKPRVL | 1 |
| | | | | | PGTLGGQALHLRSWLLSRQ | |
| 112 | Q9UQ90 | SPG7_HUMAN | SPG7 CAR | TRANSIT 1 . . . 43 | MAVLLLLLRALRRGPGPGPRPL | 2 |
| | | | CMAR | | WGPGPAWSPGFPARPGRGRP | |
| | | | PGN | | Y | |
| 113 | Q9BVV7 | TIM21_HUMAN | TIMM21 | TRANSIT 1 . . . 18 | MICTFLRAVQYTEKLHRS | 1 |
| | | | C18orf55 | | | |
| | | | TIM21 | | | |
| | | | HSPC154 | | | |
| 114 | Q9BSF4 | TIM29_HUMAN | TIMM29 | TRANSIT 1 . . . 31 | MAAAALRRFWSRRRAEAGDAV | 1 |
| | | | c19orf52 | | VAKPGVWARL | |
| 115 | Q3ZCQ8 | TIM50_HUMAN | TIMM50 | TRANSIT 1 . . . 44 | MAASAAVFSRLRSGLRLGSRG | 1 |
| | | | TIM50 | | LCTRLATPPRRAPDQAAEIGSR | |
| | | | PRO1512 | | G | |
| 116 | Q6PI78 | TMM65_HUMAN | TMEM65 | TRANSIT 1 . . . 61 | MSRLLPLLRSRTARSLRPGPAA | 3 |
| | | | | | AAAPRPPSWCCCGRGLLALAP | |
| | | | | | PGGLPGGPRRLGTHPKKE | |
| 117 | Q9BUB7 | TMM70_HUMAN | TMEM70 | TRANSIT 1 . . . 81 | MLFLALGSPWAVELPLCGRRT | 2 |
| | | | | | ALCAAAALRGPRASVSRASSSS | |
| | | | | | GPSGPVAGWSTGPSGAARLLR | |
| | | | | | RPGRAQIPVYWEGYVRF | |
| 118 | P53007 | TXTP_HUMAN | SLC25A1 | TRANSIT 1 . . . 13 | MPAPRAPRALAAA | 6 |
| | | | SLC20A3 | | | |

TABLE 6
MTS Sequences of Nuclear Encoded Proteins of the Inner Membrane lacking
TM domains

| SEQ | Uniprot | | Human | | |
|---|---|---|---|---|---|
| ID: | Identifier | Entry Name | Gene | MTS | MTS Seq |
| 119 | Q9H845 | ACAD9_HUMAN | ACAD9 | TRANSIT | MSGCGLFLRTTAAARACRGLVVSTANRRLLRT |
| | | | | 1 . . . 37 | SPPVR |
| 120 | O14561 | ACPM_HUMAN | NDUFAB1 | TRANSIT | MASRVLSAYVSRLPAAFAPLPRVRMLAVARPL |
| | | | | 1 . . . 68 | STALCSAGTQTRLGTLQPALVLAQVPGRVTQL |
| | | | | | CRQY |
| 121 | P22570 | ADRO_HUMAN | FDXR | TRANSIT | MASRCWRWWGWSAWPRTRLPPAGSTPSFC |
| | | | ADXR | 1 . . . 32 | HHF |
| 122 | 095831 | AIFM1_HUMAN | AIFM1 AIF | TRANSIT | MFRCGGLAAGALKQKLVPLVRTVCVRSPRQR |
| | | | PDCD8 | 1 . . . 54 | NRLPGNLFQRWHVPLELQMTRQM |
| 123 | P24539 | AT5F1_HUMAN | ATP5PB | TRANSIT | MLSRVVLSAAATAAPSLKNAAFLGPGVLQATR |
| | | | ATP5F1 | 1 . . . 42 | TFHTGQPHLV |
| 124 | P18859 | ATP5J_HUMAN | ATP5PF | TRANSIT | MILQRLFRFSSVIRSAVSVHLRRNIGVTAVAF |
| | | | ATP5A | 1 . . . 32 | |
| | | | ATP5J | | |
| | | | ATPM | | |
| 125 | Q99766 | ATP5S_HUMAN | DMAC2L | TRANSIT | MCCAVSEQRLTCADQMMPFGKISQQLCGVKK |
| | | | ATP5S | 1 . . . 40 | LPWSCDSRY |
| | | | ATPW | | |
| 126 | P25705 | ATPA_HUMAN | ATP5F1A | TRANSIT | MLSVRVAAAVVRALPRRAGLVSRNALGSSFIA |
| | | | ATP5A | 1 . . . 43 | ARNFHASNTHL |
| | | | ATP5A1 | | |
| | | | ATP5AL2 | | |
| | | | ATPM | | |
| 127 | P06576 | ATPB_HUMAN | ATP5F1B | TRANSIT | MLGFVGRVAAAPASGALRRLTPSASLPPAQLL |
| | | | ATP5B | 1 . . . 47 | LRAAPTAVHPVRDYA |
| | | | ATPMB | | |
| | | | ATPSB | | |
| 128 | P30049 | ATPD_HUMAN | ATP5F1D | TRANSIT | MLPAALLRRPGLGRLVRHARAY |
| | | | ATP5D | 1 . . . 22 | |
| 129 | P36542 | ATPG_HUMAN | ATP5F1C | TRANSIT | MFSRAGVAGLSAWTLQPQWIQVRNM |
| | | | ATP5C | 1 . . . 25 | |
| | | | ATP5C1 | | |
| | | | ATP5CL1 | | |
| 130 | P48047 | ATPO_HUMAN | ATP5PO | TRANSIT | MAAPAVSGLSRQVRCFSTSVVRP |
| | | | ATP50 | 1 . . . 23 | |
| | | | ATPO | | |
| 131 | P15538 | C11B1_HUMAN | CYP11B1 | TRANSIT | MALRAKAEVCMAVPWLSLQRAQAL |
| | | | S11BH | 1 . . . 24 | |
| 132 | P19099 | C11B2_HUMAN | CYP11B2 | TRANSIT | MALRAKAEVCVAAPWLSLQRARAL |
| | | | | 1 . . . 24 | |
| 133 | A1XBS5 | CBAR1_HUMAN | CIBAR1 | TRANSIT | MMRRTLENRNAQTKQLQTAVSNVEKHFGELC |
| | | | FAM92A | 1 . . . 47 | QIFAAYVRKTARLRDK |
| | | | FAM92A1 | | |
| 134 | P10809 | CH60_HUMAN | HSPD1 | TRANSIT | MLRLPTVFRQMRPVSRVLAPHLTRAY |
| | | | HSP60 | 1 . . . 26 | |
| 135 | Q8NE62 | CHDH_HUMAN | CHDH | TRANSIT | MWCLLRGLGRPGALARGALGQQQSLGARA |
| | | | | 1 . . . 29 | |
| 136 | Q9Y375 | CIA30_HUMAN | NDUFAF1 | TRANSIT | MALVHKLLRGTYFLRKFSKPTSAL |
| | | | CIA30 CGI- | 1 . . . 24 | |
| | | | 65 | | |
| 137 | O76031 | CLPX_HUMAN | CLPX | TRANSIT | MPSCGACTCGAAAVRLITSSLASAQRGISGGRI |
| | | | | 1 . . . 56 | HMSVLGRLGTFETQILQRAPLRS |
| 138 | Q9NZJ6 | COQ3_HUMAN | COQ3 | TRANSIT | MWSGRKLGSSGGWFLRVLGPGGCNTKAARP |
| | | | UG0215E0 | 1 . . . 85 | LISSAVYVKNQLSGTLQIKPGVFNEYRTIWFKS |
| | | | 5 | | YRTIFSCLNRIKSFRYPWARLY |
| 139 | Q9Y3A0 | COQ4_HUMAN | COQ4 CGI- | TRANSIT | MATLLRPVLRRLCGLPGLQRPAAEMPLRAR |
| | | | 92 | 1 . . . 30 | |
| 140 | Q5HYK3 | COQ5_HUMAN | COQ5 | TRANSIT | MAAPGSCALWSYCGRGWSRAMRGCQLLGLR |
| | | | | 1 . . . 42 | SSWPGDLLSARL |
| 141 | Q9Y2Z9 | COQ6_HUMAN | COQ6 CGI- | TRANSIT | MAARLVSRCGAVRAAPHSGPLVSWRRWS |
| | | | 10 | 1 . . . 28 | |
| 142 | Q99807 | COQ7_HUMAN | COQ7 | TRANSIT | MSCAGAAAAPRLWRLRPGARRSLSAYGRRTS |
| | | | | 1 . . . 35 | VRFR |
| 143 | O75208 | COQ9_HUMAN | COQ9 | TRANSIT | MAAAAVSGALGRAGWRLLQLRCLPVARCRQA |
| | | | C16orf49 | 1 . . . 44 | LVPRAFHASAVGL |
| | | | HSPC326 | | |
| | | | PSEC0129 | | |
| 144 | P20674 | COX5A_HUMAN | COX5A | TRANSIT | MLGAALRRCAVAATTRADPRGLLHSARTPGPA |
| | | | | 1 . . . 41 | VAIQSVRCY |
| 145 | P10606 | COX5B_HUMAN | COX5B | TRANSIT | MASRLLRGAGTLAAQALRARGPSGAAAMRSM |
| | | | | 1 . . . 31 | |
| 146 | O14548 | COX7R_HUMAN | COX7A2L | TRANSIT | MYYKFSGFTQKLAGAWASEAYSPQGLKPVVS |
| | | | COX7AR | 1 . . . 55 | TEAPPIIFATPTKLTSDSTVYDYA |
| | | | COX7RP | | |
| 47 | P05108 | CP11A_HUMAN | CYP11A1 | TRANSIT | MLAKGLPPRSVLVKGCQTFLSAPREGLGRLRV |
| | | | CYP11A | 1 . . . 39 | PTGEGAG |
| 148 | Q07973 | CP24A_HUMAN | CYP24A1 | TRANSIT | MSSPISKSRSLAAFLQQLRSPRQPPRLVTSTAY |
| | | | CYP24 | 1 . . . 35 | TS |
| 149 | Q02318 | CP27A_HUMAN | CYP27A1 | TRANSIT | MAALGCARLRWALRGAGRGLCPHGARAKAAI |
| | | | CYP27 | 1 . . . 33 | PA |
| 150 | P31327 | CPSM_HUMAN | CPS1 | TRANSIT | MTRILTAFKVVRTLKTGFGFTNVTAHQKWKFS |
| | | | | 1 . . . 38 | RPGIRL |
| 151 | P23786 | CPT2_HUMAN | CPT2 | TRANSIT | MVPRLLLRAWPRGPAVGPGAPSRPL |
| | | | CPT1 | 1 . . . 25 | |
| 152 | Q96MF6 | CQ10A_HUMAN | COQ10A | TRANSIT | MAWAGSRRVPAGTRA |
| | | | UNQ6192/ | 1 . . . 15 | |
| | | | PRO20219 | | |
| 153 | Q9H8M1 | CQ10B_HUMAN | COQ10B | TRANSIT | MAARTGHTALRRVVSGCRPKSATAAGAQAPV |
| | | | | 1 . . . 37 | RNGRYL |
| 154 | Q14154 | DELE1_HUMAN | DELE1 | TRANSIT | MWRLPGLLGRALPRTLGPSLWRVTPKSTSPD |
| | | | DELE | 1 . . . 101 | GPQTTSSTLLVPVPNLDRSGPHGPGTSGGPR |
| | | | KIAA0141 | | SHGWKDAFQWMSSRVSPNTLWDAISWGTLAV |
| | | | | | LALQLARQ |
| 155 | P40939 | ECHA_HUMAN | HADHA | TRANSIT | MVACRAIGILSRFSAFRILRSRGYICRNFTGSSA |
| | | | HADH | 1 . . . 36 | LL |
| 156 | P55084 | ECHB_HUMAN | HADHB | TRANSIT | MTILTYPFKNLPTASKWALRFSIRPLSCSSQLR |
| | | | MSTP029 | 1 . . . 33 | |
| 157 | Q9BQ95 | ECSIT_HUMAN | ECSIT | TRANSIT | MSWVQATLLARGLCRAWGGTCGAALTGTSIS |
| | | ERAL1_HUMAN | | .48 | QVPRRLPRGLHCSAAAH |
| 158 | O75616 | | ERAL1 | TRANSIT | MAAPSWRGARLVQSVLRVWQVGPHVARERVI |
| | | | HERA | 1 . . . 43 | PFSSLLGFQRRC |
| 159 | Q16134 | ETFD_HUMAN | ETFDH | TRANSIT | MLVPLAKLSCLAYQCFHALKIKKNYLPLCATRW |
| | | | | 1 . . . 33 | |
| 160 | Q9Y2C4 | EXOG_HUMAN | EXOG | TRANSIT | MAIKSIASRLRGSRRFLSGFVAGAVVGAAGAG |
| | | | ENDOGL1 | 1 . . . 41 | LAALQFFRS |
| | | | ENDOGL2 | | |
| | | | ENGL | | |
| 161 | Q05932 | FOLC_HUMAN | FPGS | TRANSIT | MSRARSHLRAALFLAAASARGITTQVAARRGL |
| | | | | 1 . . . 42 | SAWPVPQEPS |
| 162 | P50440 | GATM_HUMAN | GATM | TRANSIT | MLRVRCLRGGSRGAEAVHYIGSRLGRTLTGW |
| | | | AGAT | 1 . . . 37 | VQRTFQ |
| 163 | P34897 | GLYM_HUMAN | SHMT2 | TRANSIT | MLYFSLFWAARPLQRCGQLVRMAIRAQHS |
| | | | | 1 . . . 29 | |
| 164 | P43304 | GPDM_HUMAN | GPD2 | TRANSIT | MAFQKAVKGTILVGGGALATVLGLSQFAHYRR |
| | | | | 1 . . . 42 | KQMNLAYVKA |
| 165 | P38646 | GRP75_HUMAN | HSPA9 | TRANSIT | MISASRAAAARLVGAAASRGPTAARHQDSWN |
| | | | GRP75 | 1 . . . 46 | GLSHEAFRLVSRRDY |
| | | | HSPA9B | | |
| | | | mt-HSP70 | | |
| 166 | Q9HAV7 | GRPE1_HUMAN | GRPEL1 | TRANSIT | MAAQCVRLARRSLPALALSLRPSPRLL |
| | | | GREPEL1 | 1 . . . 27 | |
| 167 | Q8TAA5 | GRPE2_HUMAN | GRPEL2 | TRANSIT | MAVRSLWAGRLRVQRLLAWSAAWESKGWPL |
| | | | | 1 . . . 32 | PF |
| 168 | Q8N442 | GUF1_HUMAN | GUF1 | TRANSIT | MWTLVGRGWGCARALAPRATGAALLVAPGPR |
| | | | | 1 . . . 49 | SAPTLGAAPESWATDRLY |
| 169 | P22557 | HEMO_HUMAN | ALAS2 | TRANSIT | MVTAAMLLQCCPVLARGPTSLLGKVVKTHQFL |
| | | | ALASE | 1 . . . 49 | FGIGRCPILATQGPNCS |
| | | | ASB | | |
| 170 | P13196 | HEM1_HUMAN | ALAS1 | TRANSIT | MESVVRRCPFLSRVPQAFLQKAGKSLLFYAQN |
| | | | ALAS3 | 1 . . . 56 | CPKMMEVGAKPAPRALSTAAVHYQ |
| | | | ALASH | | |
| | | | OK/SW- | | |
| | | | cl. 121 | | |
| 171 | P36551 | HEM6_HUMAN | CPOX CPO | TRANSIT | MALQLGRLSSGPCWLVARGGCGGPRAWSQC |
| | | | CPX | 1 . . . 110 | GGGGLRAWSQRSAAGRVCRPPGPAGTEQSR |
| | | | | | GLGHGSTSRGGPWVGTGLAAALAGLVGLATA |
| | | | | | AFGHVQRAEMLPKTSGTRA |
| 172 | P22830 | HEMH_HUMAN | FECH | TRANSIT | MRSLGANMAAALRAAGVLLRDPLASSSWRVC |
| | | | | 1 . . . 54 | QPWRWKSGAAAAAVTTETAQHAQ |
| 173 | Q14197 | ICT1_HUMAN | MRPL58 | TRANSIT | MAATRCLRWGLSRAGVWLLPPPARCPRRA |
| | | | DS1 ICT1 | 1 . . . 29 | |
| 174 | O75600 | KBL_HUMAN | GCAT KBL | TRANSIT | MWPGNAWRAALFWVPRGRRAQ |
| | | | | 1 . . . | |
| | | | | 21 | |
| 175 | P17540 | KCRS_HUMAN | CKMT2 | TRANSIT | MASIFSKLLTGRNASLLFATMGTSVLTTGYLLN |
| | | | | 1 . . . 39 | RQKVCA |
| 176 | P12532 | KCRU_HUMAN | CKMT1A | TRANSIT | MAGPFSRLLSARPGLRLLALAGAGSLAAGFLL |
| | | | CKMT; | 1 . . . 39 | RPEPVRA |
| | | | CKMT1B | | |
| | | | CKMT | | |
| 177 | Q9H9P8 | L2HDH_HUMAN | L2HGDH | TRANSIT | MVPALRYLVGACGRARGLFAGGSPGACGFAS |
| | | | C14orf160 | 1 . . . 51 | GRPRPLCGGSRSASTSSFDI |
| 178 | Q86WU2 | LDHD_HUMAN | LDHD | TRANSIT | MARLLRSATWELFPWRGYCSQKAKGELCRDF |
| | | | | 1 . . . 52 | VEALKAVVGGSHVSTAAVVRE |
| 179 | Q8WWC4 | MAIP1_HUMAN | MAIP1 | TRANSIT | MALAARLLPQFLHSRSLPCGAVRLRTPAVAEV |
| | | | C2orf47 | 1 . . . 96 | RLPSATLCYFCRCRLGLGAALFPRSARALAAS |
| | | | | | ALPAQGSRWPVLSSPGLPAAFASFPACPQRSY |
| 180 | Q8IYU8 | MICU2_HUMAN | MICU2 | TRANSIT | MAAAAGSCARVAAWGGKLRRGL |
| | | | EFHA1 | 1 . . . 22 | |
| 181 | Q10713 | MPPA_HUMAN | PMPCA | TRANSIT | MAAVVLAATRLLRGSGSWGCSRLRFGPPAYR |
| | | | INPP5E | 1 . . . 33 | RF |
| | | | KIAA0123 | | |
| | | | MPPA | | |
| 182 | O75439 | MPPB_HUMAN | PMPCB | TRANSIT | MAAAAARVVLSSAARRRLWGFSESLLIRGAAG |
| | | | MPPB | 1 . . . 43 | RSLYFGENRLR |
| 183 | Q9BT17 | MTG1_HUMAN | MTG1 | TRANSIT | MRLTPRAL |
| | | | GTPBP7 | 1 . . . 8 | |
| 184 | O00746 | NDKM_HUMAN | NME4 | TRANSIT | MGGLFWRSALRGLRCGPRAPGPSLLVRH |
| | | | NM23D | 1 . . . 28 | |
| 185 | Q16795 | NDUA9_HUMAN | NDUFA9 | TRANSIT | MAAAAQSRVVRVLSMSRSAITAIATSVCHGPP |
| | | | NDUFS2L | 1 . . . 35 | CRQ |
| 186 | 095299 | NDUAA_HUMAN | NDUFA10 | TRANSIT | MALRLLKLAATSASARVVAAGAQRVRGIHSSV |
| | | | | 1 . . . 35 | QCK |
| 187 | 095178 | NDUB2_HUMAN | NDUFB2 | TRANSIT | MSALTRLASFARVGGRLFRSGCARTAGDGGV |
| | | | | 1 . . . 33 | RH |
| 188 | Q8N183 | NDUF2_HUMAN | NDUFAF2 | TRANSIT | (unknown) |
| | | | NDUFA12L | 1 . . . ? | |
| 189 | Q5TEU4 | NDUF5_HUMAN | NDUFAF5 | TRANSIT | MLRPAGLWRLCRRPWAARVPAENLGRREVTS |
| | | | C20orf7 | 1 . . . 36 | GVSPR |
| 190 | Q330K2 | NDUF6_HUMAN | NDUFAF6 | TRANSIT | MAASAHGSVWGPLRLGIPGLCCRRPPLGLYAR |
| | | | C8orf38 | 1 . . . 44 | MRRLPGPEVSGR |
| 191 | P28331 | NDUS1_HUMAN | NDUFS1 | TRANSIT | MLRIPVRKALVGLSKSPKGCVRT |
| | | | | 1 . . . 23 | |
| 192 | O75306 | NDUS2_HUMAN | NDUFS2 | TRANSIT | MAALRALCGFRGVAAQVLRPGAGVRLPIQPSR |
| | | | | 1 . . . 33 | G |
| 193 | O75489 | NDUS3_HUMAN | NDUFS3 | TRANSIT | MAAAAVARLWWRGILGASALTRGTGRPSVLLL |
| | | | | 1 . . . 36 | PVRR |
| 194 | 043181 | NDUS4_HUMAN | NDUFS4 | TRANSIT | MAAVSMSVVLRQTLWRRRAVAVAALSVSRVP |
| | | | | 1 . . . 42 | TRSLRTSTWRL |
| 195 | O75380 | NDUS6_HUMAN | NDUFS6 | TRANSIT | MAAAMTFCRLLNRCGEAARSLPLGARCF |
| | | | | 1 . . . 28 | |
| 196 | O75251 | NDUS7_HUMAN | NDUFS7 | TRANSIT | MAVLSAPGLRGFRILGLRSSVGPAVQARGVHQ |
| | | | | 1 . . . 38 | SVATDG |
| 197 | O00217 | NDUS8_HUMAN | NDUFS8 | TRANSIT | MRCLTTPMLLRALAQAARAGPPGGRSLHSSAV |
| | | | | 1 . . . 34 | AA |
| 198 | P49821 | NDUV1_HUMAN | NDUFV1 | TRANSIT | MLATRRLLGWSLPARVSVRF |
| | | | UQOR1 | 1 . . . 20 | |
| 199 | P19404 | NDUV2_HUMAN | NDUFV2 | TRANSIT | MFFSAALRARAAGLTAHWGRHVRNLHKTVMQ |
| | | | | 1 . . . 32 | N |
| 200 | P56181 | NDUV3_HUMAN | NDUFV3 | TRANSIT | MAAPCLLRQGRAGALKTMLQEAQVFRGLAST |
| | | | | 1 . . . 34 | VSL |
| 201 | Q14249 | NUCG_HUMAN | ENDOG | TRANSIT | MRALRAGLTLASGAGLGAVVEGWRRRREDAR |
| | | | | 1 . . . 48 | AAPGLLGRLPVLPVAAA |
| 202 | P00480 | OTC_HUMAN | OTC | TRANSIT | MLFNLRILLNNAAFRNGHNFMVRNFRCGQPLQ |
| | | | | 1 . . . 32 | |
| 203 | Q32NB8 | PGPS1_HUMA | PGS1 | TRANSIT | MAVAAAAAAGPVFWRRLLGLLPGRPGLA |
| | | | | 1 . . . 28 | |
| 204 | P30405 | PPIF_HUMAN | PPIF CYP3 | TRANSIT | MLALRCGSRWLGLLSVPRSVPLRLPAARA |
| | | | | 1 . . . 29 | |
| 205 | O43272 | PROD_HUMAN | PRODH | TRANSIT | (unknown) |
| | | | PIG6 POX2 | 1 . . . ? | |
| | | | PRODH2 | | |
| 206 | Q96EY7 | PTCD3_HUMA | PTCD3 | TRANSIT | MAVVSAVRWLGLRSRLGQPLTGRRAGLCEQA |
| | | | MRPS39 | 1 . . . 37 | RSCRFY |
| | | | TRG15 | | |
| 207 | Q8WUK0 | PTPM1_HUMA | PTPMT1 | TRANSIT | MAATALLEAGLARVLFYPTLLYTLFRG |
| | | | MOSP | 1 . . . 27 | |
| | | | PLIP | | |
| | | | PNAS-129 | | |
| 208 | P31930 | QCR1_HUMAN | UQCRC1 | TRANSIT | MAASVVCRAATAGAQVLLRARRSPALLRTPAL |
| | | | | 1 . . . 34 | RS |
| 209 | P22695 | QCR2_HUMAN | UQCRC2 | TRANSIT | MKLLTRAGSFSRFY |
| | | | | 1 . . . 14 | |
| 210 | P07919 | QCR6_HUMAN | UQCRH | TRANSIT | MGLEDEQKMLTES |
| | | | | 1 . . . 13 | |
| 211 | Q96151 | RCC1L_HUMAN | RCC1L | TRANSIT | MALVALVAGARLGRRLSGPGLGRGHWTAAGR |
| | | | WBSCR16 | 1 . . . 31 | |
| 212 | Q9BYD6 | RM01_HUMAN | MRPL1 | TRANSIT | MAAAVRCMGRALIHHQRHSLSKMVYQTSLCS |
| | | | BM-022 | 1 . . . 50 | CSVNIRVPNRHFAAATKSA |
| 213 | Q5T653 | RM02_HUMAN | MRPL2 | TRANSIT | MALCALTRALRSLNLAPPTVAAPAPSLFPAAQ |
| | | | CGI-22 | 1 . . . 60 | MMNNGLLQQPSALMLLPCRPVLTSVALN |
| 214 | P09001 | RM03_HUMAN | MRPL3 | TRANSIT | MPGWRLLTQVGAQVLGRLGDGLGAALGPGNR |
| | | | MRL3 | 1 . . . 40 | THIWLFVRG |
| | | | RPML3 | | |
| 215 | Q9BYD2 | RM09_HUMAN | MRPL9 | TRANSIT | MAAPVVTAPGRALLRAGAGRLLRGGVQELLRP |
| | | | | 1 . . . 52 | RHEGNAPDLACNFSLSQNRG |
| 216 | Q7Z7H8 | RM10_HUMAN | MRPL10 | TRANSIT | MAAAVAGMLRGGLLPQAGRLPTLQTVRY |
| | | | MRPL8 | 1 . . . 28 | |
| | | | RPML8 | | |
| 217 | Q9Y3B7 | RM11_HUMAN | MRPL11 | TRANSIT | MSKLGRAARG LRKPEVGGVI RAIVRAGLAM |
| | | | CGI-113 | 1 . . . 43 | PGPPLGPVLG QRG |
| 218 | P52815 | RM12_HUMAN | MRPL12 | TRANSIT | MLPAAARPLWGPCLGLRAAAFRLARRQVPCV |
| | | | MRPL7 | 1 . . . 45 | CAVRHMRSSGHQRC |
| | | | RPML12 | | |
| 219 | Q6P1L8 | RM14_HUMAN | MRPL14 | TRANSIT | MAFFTGLWGPFTCVSRVLSHHCFSTTGSLS |
| | | | MRPL32 | 1 . . . 30 | |
| | | | RPML32 | | |
| 220 | Q9P015 | RM15_HUMAN | MRPL15 | TRANSIT | MAGPLQGGGARALDLLRGLPR |
| | | | HSPC145 | 1 . . . 21 | |
| 221 | Q9NX20 | RM16_HUMAN | MRPL16 | TRANSIT | MWRLLARASAPLLRVPLSDSWALLPASAG |
| | | | PNAS-111 | 1 . . . 29 | |
| 222 | Q9NRX2 | RM17_HUMAN | MRPL17 | TRANSIT | (unknown) |
| | | | LIP2 | 1 . . . 8 | |
| 223 | Q9HOU6 | RM18_HUMAN | MRPL18 | TRANSIT | MALRSRFWGLFSVCRNPGORF |
| | | | HSPC071 | 1 . . . 21 | |
| 224 | P49406 | RM19_HUMAN | MRPL19 | TRANSIT | MAACIAAGHWAAMGLGRSFQAARTLLPPPAS! |
| | | | KIAA0104 | 1 . . . 43 | ACRVHAGPVRQ |
| | | | MRPL15 | | |
| 225 | Q9BYC9 | RM20_HUMAN | MRPL20 | TRANSIT | MVFLTAQLWLRNRVTDRYFRIQEVLKHARH |
| | | | | 1 . . . 30 | |
| 226 | Q7Z2W9 | RM21_HUMAN | MRPL21 | TRANSIT | MAASSLTVTLGRLASACSHSILRPSGPGAASL |
| | | | | 1 . . . 39 | WSASRRF |
| 227 | Q9NWU5 | RM22_HUMAN | MRPL22 | TRANSIT | MAAAVLGQLGALWIHNLRSRGKLALGVLPQSYI |
| | | | MRPL25 | 1 . . . 40 | HTSASLD |
| | | | RPML25 | | |
| | | | HSPC158 | | |
| 228 | Q96A35 | RM24_HUMAN | MRPL24 | TRANSIT | MRLSALLAL |
| | | | | 1 . . . 9 | |
| 229 | Q9PM9 | RM27_HUMAN | MRPL27 | TRANSIT | MASVVLALRTRTAVTSLLSPTPATALAVRY |
| | | | HSPC250 | 1 . . . 30 | |
| 230 | Q13084 | RM28_HUMAN | MRPL28 | TRANSIT | MPLHKYPVWLWKRLQLREGICSRLPGHYLRSL |
| | | | MAAT1 | 1 . . . 55 | EEERTPTPVHYRPHGAKFKINPK |
| 231 | Q8TCC3 | RM30_HUMAN | MRPL30 | TRANSIT | MAGILRLVVQWPPGRLQTVTKGVESLICTDWIR |
| | | | MRPL28 | 1 . . . 34 | H |
| | | | RPML28 | | |
| | | | HSPC249 | | |
| 232 | Q9BYC8 | RM32_HUMAN | MRPL32 | TRANSIT | MAMAMLVLVVSPWSAARGVLRNYWERLLRKL |
| | | | HSPC283 | 1 . . . 55 | PQSRPGFPSPPWGPALAVQGPAMF |
| 233 | O75394 | RM33_HUMAN | MRPL33 | TRANSIT | |
| | | | C2orf1 | 1 . . . 8 | |
| 234 | Q9BQ48 | RM34_HUMAN | MRPL34 | TRANSIT | MAVLAGSLLGPTSRSAALLGGRWLQPRAWLG |
| | | | | 1 . . . 46 | FPDAWGLPTPQQARG |
| 235 | Q9NZE8 | RM35_HUMAN | MRPL35 | TRANSIT | MAASAFAGAVRAASGILRPLNILASSTYRNCVK |
| | | | BM-007 | 1 . . . 46 | NASLISALSTGRF |
| 236 | Q9POJ6 | RM36_HUMAN | MRPL36 | TRANSIT | MANLFIRKMVNPLLYLSRHTVKPRALSTFLFGSI |
| | | | BRIP1 | 1 . . . 36 | RG |
| 237 | Q9BZE1 | RM37_HUMAN | MRPL37 | TRANSIT | MALASGPARRALAGSGQLGLGGFGAPRRG |
| | | | MRPL2 | 1 . . . 29 | |
| | | | RPML2 | | |
| | | | HSPC235 | | |
| 238 | Q96DV4 | RM38_HUMAN | MRPL38 | TRANSIT | MAAPWWRAALCECRRWRGFSTSAVLG |
| | | | HSPC262 | 1 . . . 26 | |
| 239 | Q9NQ50 | RM40_HUMAN | MRPL40 | TRANSIT | MTASVLRSISLALRPTSGLLGTWQTQLRETHQ |
| | | | NLVCF | 1 . . . 46 | RASLLSFWELIPMR |
| | | | URIM | | |
| 240 | Q8IXM3 | RM41_HUMAN | MRPL41 | TRANSIT | MGVLAAAARCLVR |
| | | | BMRP | 1 . . . 13 | |
| | | | MRPL27 | | |
| | | | RPML27 | | |
| | | | PIG3 | | |
| 241 | Q9Y6G3 | RM42_HUMAN | MRPL42 | TRANSIT | MAVAAVKWVMSKRTILKHLFPVQNGALYCVCH |
| | | | MRPL31 | 1 . . . 32 | |
| | | | MRPS32 | | |
| | | | RPML31 | | |
| | | | HSPC204 | | |
| | | | PTD007 | | |
| 242 | Q8N983 | RM43_HUMAN | MRPL43 | TRANSIT | (unknown) |
| | | | | 1 . . . ? | |
| 243 | Q9H9J2 | RM44_HUMAN | MRPL44 | TRANSIT | MASGLVRLLQQGHRCLLAPVAPKLVPPVRG |
| | | | | 1 . . . 30 | |
| 244 | Q9BRJ2 | RM45_HUMAN | MRPL45 | TRANSIT | MRLFSYPTLFTLSSLPGGLC |
| | | | | 1 . . . 20 | |
| 245 | Q9H2VV6 | RM46_HUMAN | MRPL46 | TRANSIT | MAAAPVRRTLLGVAGGWRRFERLWAGSLSSR |
| | | | C15orf4 | 1 . . . 53 | SLALAAAPSSNGSPWRLLGALCL |
| | | | LIECG2 | | |
| 246 | Q9HD33 | RM47_HUMAN | MRPL47 | TRANSIT | MAAAGLALLCRRVSSALKSSRSLITPQVPACTG |
| | | | NCM1 CGI- | 1 . . . 54 | FFLSLLPKSTPNVTSFHQYRL |
| | | | 204 | | |
| 247 | Q96GC5 | RM48_HUMAN | MRPL48 | TRANSIT | MSGTLEKVLCLRNNTIFKQAFSLLRFRT |
| | | | CGI-118 | 1 . . . 28 | |
| | | | HSPC290 | | |
| 248 | Q4U2R6 | RM51_HUMAN | MRPL51 | TRANSIT | MAGNLLSGAGRRLWDWVPLACRSFSLGVPRL |
| | | | MRP64 | 1 . . . 31 | |
| | | | CDA09 | | |
| | | | HSPC241 | | |
| 249 | Q86TS9 | RM52_HUMAN | MRPL52 | TRANSIT | MAALGTVLFTGVRRLHCSVAAWA |
| | | | | 1 . . . 23 | |
| 250 | Q96EL3 | RM53_HUMAN | MRPL53 | TRANSIT | (unknown) |
| | | | | 1 . . . ? | |
| 251 | Q6P161 | RM54_HUMAN | MRPL54 | TRANSIT | MATKRLFGATRTWA |
| | | | | 1 . . . 14 | |
| 252 | Q7Z7F7 | RM55_HUMAN | MRPL55 | TRANSIT | MAAVGSLLGRLRQSTVKATGPALRRLHTSSW |
| | | | UNQ5835/ | 1 . . . 33 | RA |
| | | | PRO19675 | | |
| 253 | Q9Y2R9 | RT07_HUMAN | MRPS7 | TRANSIT | MAAPAVKVARGWSGLALGVRRAVLQLPGLTQ |
| | | | | 1 . . . 37 | VRWSRY |
| 254 | P82933 | RT09_HUMAN | MRPS9 | TRANSIT | MAAPCVSYGGAVSYRLLLWGRGSLARKQGLW |
| | | | RPMS9 | 1 . . . 51 | KTAAPELQTNVRSQILRLRH |
| 255 | P82912 | RT11_HUMAN | MRPS11 | TRANSIT | MQAVRNAGSRFLRSWTWPQTAGRVVARTPA |
| | | | RPMS11 | 1 . . . 39 | GTICTGARQ |
| | | | HCC2 | | |
| 256 | O15235 | RT12_HUMAN | MRPS12 | TRANSIT | MSWSGLLHGLNTSLTCGPALVPRLWATCS |
| | | | RPMS12 | 1 . . . 29 | |
| | | | RPSM12 | | |
| 257 | P82914 | RT15_HUMAN | MRPS15 | TRANSIT | MLRVAWRTLSLIRTRAVTQVLVPGLPGGGSAK |
| | | | RPMS15 | 1 . . . 57 | FPFNQWGLQPRSLLLQAARGYVVRK |
| | | | DC37 | | |
| 258 | Q9Y3D3 | RT16_HUMAN | MRPS16 | TRANSIT | MVHLTTLLCKAYRGGHLTIRLALGGCTNRPFYR |
| | | | RPMS16 | 1 . . . 34 | |
| | | | CGI-132 | | |
| 259 | Q9Y2R5 | RT17_HUMAN | MRPS17 | TRANSIT | MSVVRSSVHARWIVGKVIGT |
| | | | RPMS17 | 1 . . . 20 | |
| | | | HSPC011 | | |
| 260 | Q9NVS2 | RT18A_HUMAN | MRPS18A | TRANSIT | MAALKALVSGCGRLLRGLLAGPAATSWSRLPA |
| | | | | 1 . . . 34 | RG |
| 261 | Q9Y676 | RT18B_HUMAN | MRPS18B | TRANSIT | MAASVLNTVLRRLPMLSLFRGSHRVQVPLQTL |
| | | | C6orf14 | 1 . . . 35 | CTK |
| | | | HSPC183 | | |
| | | | PTD017 | | |
| 262 | Q9Y3D5 | RT18C_HUMAN | MRPS18C | TRANSIT | MAVAVCGGLGRKKLTHLVTAAVSLTHPGTHTV |
| | | | CGI-134 | 1 . . . 40 | LWRRGC |
| 263 | Q96EL2 | RT24_HUMAN | MRPS24 | TRANSIT | MAASVCSGLLGPRVLSWSRELPCAWRALHTS |
| | | | HSPC335 | 1 . . . 35 | PVCA |
| 264 | Q9BYN8 | RT26_HUMAN | MRPS26 | TRANSIT | MLRALSRLGAGTPCRPRAPLVLPARGR |
| | | | C20orf193 | 1 . . . 27 | |
| | | | RPMS13 | | |
| 265 | Q92552 | RT27_HUMAN | MRPS27 | TRANSIT | MAASIVRRGMLLARQVVLPQLSPAGKRYLLSS |
| | | | KIAA0264 | 1 . . . 36 | AYVD |
| 266 | Q9Y2Q9 | RT28_HUMAN | MRPS28 | TRANSIT | MAALCRTRAVAAESHFLRVFLFFRPFRGVGTE |
| | | | MRPS35 | 1 . . . 71 | SGSESGSSNAKEPKTRAGGFASALERHSELLQ |
| | | | HSPC007 | | KVEPLQK |
| 267 | P51398 | RT29_HUMAN | DAP3 | TRANSIT | MMLKGITRLISRIHKLDPGRF |
| | | | MRPS29 | 1 . . . 21 | |
| 268 | Q92665 | RT31_HUMAN | MRPS31 | TRANSIT | MFPRVSTFLPLRPLSRHPLSSGSPETSAAAIML |
| | | | IMOGN38 | 1 . . . 65 | LTVRHGTVRYRSSALLARTKNNIQRYFGTNSV |
| 269 | P82673 | RT35_HUMAN | MRPS35 | TRANSIT | JAAAALPAWLSLQSRARTLRAFSTAVY |
| | | | MRPS28 | 1 . . . 27 | |
| | | | HDCMD11 | | |
| | | | P MDS023 | | |
| | | | PSEC0213 | | |
| 270 | P31040 | SDHA_HUMAN | SDHA | TRANSIT | MSGVRGLSRLLSARRLALAKAWPTVLQTGTRG |
| | | | SDH2 | 1 . . . 42 | FHFTVDGNKR |
| | | | SDHF | | |
| 271 | P21912 | SDHB_HUMAN | SDHB SDH | TRANSIT | MAAVVALSLRRRLPATTLGGACLQASRG |
| | | | SDH1 | 1 . . . 28 | |
| 272 | Q9Y6E7 | SIR4_HUMAN | SIRT4 | TRANSIT | MKMSFALTFRSAKGRWIANPSQPCSKAS |
| | | | SIR2L4 | 1 . . . 28 | |
| 273 | Q9Y6N5 | SQOR_HUMAN | SQOR | TRANSIT | MVPLVAVVSGPRAQLFACLLRLGTQQVGPLQL |
| | | | SQRDL | 1 . . . 38 | HTGASH |
| | | | CGI-44 | | |
| 274 | Q9UJZ1 | STML2_HUMAN | STOML2 | TRANSIT | MLARAARGTGALLLRGSLLASGRAPRRA |
| | | | SLP2 | 1 . . . 28 | |
| | | | HSPC108 | | |
| 275 | Q8IZJ6 | TDH_HUMAN | TDH | TRANSIT | (unknown) |
| | | | SDR14E1P | 1 . . . ? | |
| 276 | Q5T1C6 | THEM4_HUMAN | THEM4 | TRANSIT | MLRSCAARLRTLGALCLPPVGRRLPGSEPRPE |
| | | | CTMP | 1 . . . 36 | LRSF |
| 277 | O43615 | TIM44_HUMAN | TIMM44 | TRANSIT | MAAAALRSGWCRCPRRCLGSGIOFL |
| | | | MIMT44 | 1 . . . 25 | |
| | | | TIM44 | | |
| 278 | Q12931 | TRAP1_HUMAN | TRAP1 | TRANSIT | MARELRALLLWGRRLRPLLRAPALAAVPGGKP |
| | | | HSP75 | 1 . . . 59 | ILCPRRTTAQLGPRRNPAWSLQAGRLF |
| 279 | Q6DKK2 | TTC19_HUMAN | TTC19 | TRANSIT | MFRLLSWSLGRGFLRAAGRRCRGCSARLLPG |
| | | | | 1 . . . 70 | LAGGPGPEVQVPPSRVAPHGRGPGLLPLLAAL |
| | | | | | AWFSRPA |
| 280 | Q9BRT2 | UQCC2_HUMAN | UQCC2 | TRANSIT | MAASRYRRFLKLC |
| | | | C6orf125 | 1 . . . 13 | |
| | | | MNF1 | | |

### N-Terminal Export

Applicants propose that a significant proportion of allotopically expressed ND1 does not fold/assemble correctly in the inner membrane, where the short N- and C-termini should both face the intermembrane space. The identified “matrix export signal” derived from OXA1L has been shown to facilitate the export of appropriate transmembrane regions of a mature inner membrane protein from the matrix to the IMS, following MTS cleavage. Appending this sequence after the MTS signal, but upstream of the first transmembrane region of ND1 may facilitate orientation of the N-terminus toward the IMS.

### Extra TM1 (ABCBA 1-165)

The region following the MTS (approximately up until the second TM helix) has been shown to specifically route multi-pass membrane proteins to the mitochondrial inner membrane and is required for ABCBA. In the absence of this region, the protein will accumulate in the matrix. This additional sequence is also shown to facilitate similar inner-membrane targeting of proteins which would otherwise be recruited to the ER and assemble into other organelle membranes.

### Translational Stalling

The allotopically-expressed genes described are codon-optimized, and utilize the most preferred codons at nearly every position, which can facilitate robust and rapid translation of the polypeptide. However, in nuclear-encoded proteins, including those which are targeted to the mitochondria, codon use is more normally distributed. Many of these proteins, membrane proteins especially, have an overrepresentation of “rare” codons near the N-terminus and in regions surrounding highly hydrophobic membrane-spanning segments. The presence of rare codons in these positions is often conserved and believed to participate in protein folding and chaperone recruitment through modulation of translational speed. The introduction of “rare” codons into otherwise rapidly translated regions may help to slow translation dynamics and thus increase the yield of high-quality mitochondrially-targeted proteins. Rare codons can also be placed downstream of (i.e., c-terminal to) hydrophobic sequence regions, such that the nascent peptide may electrostatically interact with the ribosome surface. Such interactions can facilitate formation of transient, stable secondary structures and to prevent aggregation of highly hydrophobic proteins upon emergence from the ribosome exit tunnel. Translational slowing upon emergence of hydrophobic sequence regions can also facilitate recognition by and interaction with protein chaperones such as Hsp70 and Hsp90.

### PUMILIO

Because PUM binding is believed to cause ribosome stalling, Applicant proposed that inclusion of an upstream PUM binding site between the RBS and the TSS/start of the MTS coding sequence could prevent or inhibit translation elongation (i.e., as a negative control and evidence of PUM binding).

Previous preliminary testing has suggested, however, that upstream PUM binding, and thus translation stalling, can be beneficial for allotopic expression, potentially delaying translation of the transgenic mRNA until it has been trafficked to the vicinity of the mitochondrion. Furthermore, studies suggests that rapid translation can be detrimental for the expression of high-quality heterologous protein products, particularly because of the relationship between translation dynamics and nascent chain folding.

Because the allotopic constructs described herein predominantly utilize the most frequent codons for each amino acid (linked to translation speed), PUM domains may also be beneficial for slowing translation speed if interspersed throughout the CDS (as is also common). Instead, previous preliminary testing revealed improved expression, possibly due to ribosome stalling (which may facilitate localized translation) or slowing the translational tempo of mRNAs which may otherwise translate very rapidly due to high-frequency codon optimization.

In embodiments, compositional similarity to endogenous genes is used to:
avoid repression of transcription,
avoid immune reaction/foreign nucleic acid response, and
mimic the natural periodicity of translation speed.

### Hydrophobicity Reduction

The highly hydrophobic nature of mitochondrially-encoded subunits is believed to hinder mitochondrial import of subunits translated in the cytosol, thus promoting retention of these genes in the mitochondrial genome.

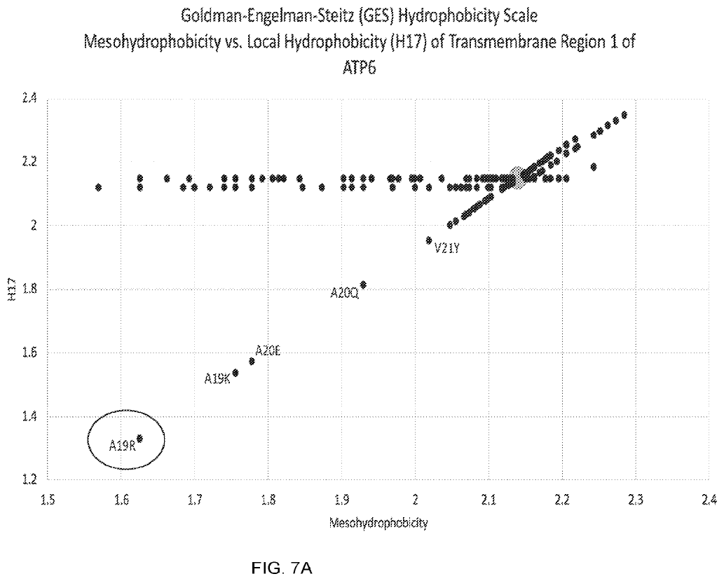

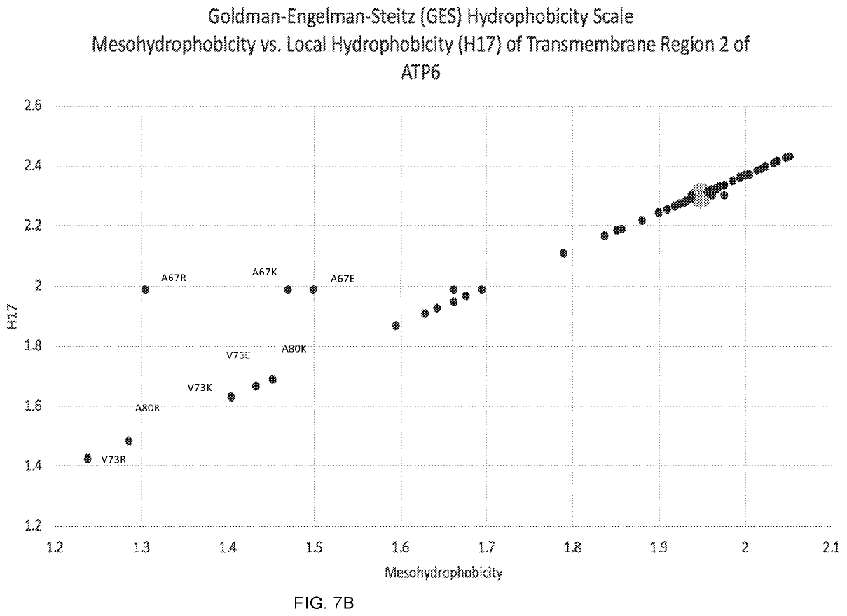

For example, the average maximum hydrophobicity of a sample of mitochondrial proteins (or proteins spanning the MIM, TM domain containing mitochondrial proteins, etc.) across the first X amino acids (or first TM domain, or MesoH, etc.) is found to be a numerical value using the hydrophobicity scale and any corresponding window size. However, mtDNA-encoded subunits demonstrate both high maximum local hydrophobicities across (same region measured in controls) and higher overall hydrophobicities than many nuclear-encoded subunits, (see, e.g., FIG. 7A showing clustering of MesoH and H17 of mito subunits as compared to nuclear comparables).

To facilitate import of exogenously expressed subunits, the present invention describes targeted amino acid substitutions within hydrophobic transmembrane regions of the allotopic protein, the effect being a reduction in local hydrophobicity without functional impairment of the protein product. Accordingly, in embodiments, annotated transmembrane regions of mitochondrial protein subunits are evaluated for overall hydrophobicity (MesoH) as well as maximal local hydrophobicity (H17), a measure of local hydrophobicity across a 17-residue peptide window, which approximates the peptide length spanning a biological membrane. The hydrophobicity of amino acid residues is ranked using the Goldman-Engelman-Seitz (GES) hydrophobicity scale.

In embodiments, hydrophobic transmembrane regions of mitochondrial subunits are evaluated computationally by substitution of each amino acid position within the region with each of the other standard 19 amino acids. Amino acid substitutions may be evaluated for suitability on the basis of the local (H17) and global (MesoH) hydrophobicity-reducing effects of a given residue substitution. Examples include:
A substitution within a transmembrane region of a protein wherein the maximum H17 is above 2.5, as measured by the GES scale;
A substitution which reduces the maximum H17 of a transmembrane region to below 2.5, as measured by the GES scale.

Substitutions can be further be characterized using software to predict the deleterious effect of a position-specific substitution on protein function. Such predictions can use local (e.g., BLocks SUbstitution Matrix, BLOSUM) or global (e.g., Point Accepted Mutation, PAM) evolutionary conservation matrices (or derivatives such as Dayhoff mutation data matrix) to demonstrate positional and functional residue conservation across species homologs of a protein or domain, in addition to the frequency of substitution with each of the other 19 amino acids. Such matrices may be used in this way to filter out substitutions which are predicted to have deleterious impact, for example, if a residue shows a high degree of conservation.

A functional impact prediction threshold may thus be established to exclude hydrophobicity-reducing substitutions which are predicted to adversely affect protein function, for example, through exclusion of highly conserved residues with a mutation frequency below an arbitrary threshold. Other methods to exclude potentially harmful residue substitutions include evaluation of annotated and predicted active sites and ligand binding regions.

Methods can also include consideration of physiochemical properties such as hydrophobicity, aromaticity and charge, which often contribute to protein-protein or protein-ligand interactions. Substitutions annotated to be associated with human disease, or those annotated with adverse functional implications are excluded. Residue substitutions within a defined functional impact threshold which also demonstrate effective reduction of H17 and/or MesoH are selected candidates.

Further, candidate substitutions may be evaluated for risk of structural protein perturbation through computational modeling of residue substitutions and measurement of the resulting deviation from the established wild-type crystal structure. Evaluation metrics may include changes to: phi/psi angles within transmembrane helices; distance between residue side chains; proximity of atoms to neighboring sidechains; regional hydrophobic tendency; interaction with membrane lipids; ligand interactions; intermolecular bonding and interfacial residues.

Accordingly, in embodiments, a transmembrane region may have one or more amino acid substitutions introduced which are predicted to reduce hydrophobicity without any deleterious effect on protein function.

Embodiments include allotopic expression of one or more mtDNA genes to compensate for a functional deficiency arising from mtDNA damage and/or mutations in mitochondrial genes. In aspects, the mtDNA gene is a complex I subunit and/or assembly factor selected from MT-ND1, MT-ND2, MT-ND3, MT-ND4L, MT-ND4, MT-ND5, MT-ND6, NDUFA6, NDUFA3, NUBPL, NDUFS3, NDUFV3, NDUFV2, DMAC1, AIFM1, NDUFS4, OXA1L, NDUFA13, NDUFAF5, NDUFAF4, NDUFA4, TIMM21, NDUFA1, NDUFB4, NDUFA8, NDUFAF8, NDUFB1, NDUFS6, NDUFA2, NDUFB6, NDUFB9, NDUFB11, WDR93, NDUFC1, DMAC2, NDUFA7, NDUFAF1, NDUFB8, NDUFAF3, NDUFAF2, NDUFAF7, TAFAZZIN, NDUFB7, NDUFAB1, NDUFB2, TMEM126B, TMEM126A, NDUFC2-KCTD14, NDUFA5, NDUFC2, NDUFA10, NDUFAF6, NDUFV1, TMEM186, BCS1L, NDUFB5, ACAD9, NDUFA11, NDUFS5, NDUFS2, NDUFB10, NDUFA12, NDUFS7, NDUFB3, ECSIT, COA1, FOXRED1, NDUFA9, NDUFS8 and NDUFS1.

In aspects, the mtDNA gene is a complex III subunit and/or assembly factor selected from MT-CYB, UQCC2, TTC19, UQCRB, SLC25A33, UQCRH, UQCR10, UQCRFS1, CYC1, LYRM7, UQCC1, UQCRC2, UQCRQ, UQCC3, BCS1L, C12orf73, UQCRC1, UQCR11 and UQCRHL.

In aspects, the mtDNA gene is a complex IV subunit and/or assembly factor selected from MT-CO1, MT-CO2, MT-CO3, SURF1, COX8C, FASTKD3, COX412, PET117, COX7C, COX5A, COX7B, OXA1L, COX14, COX7A2L, COA3, NDUFA4, COX19, COX7A1, TIMM21, COA5, SCO2, UQCRFS1, COX16, COX8A, SURF1, SCO1, UQCRC2, COX18, COX6C, COA8, C15orf48, COX6A2, COA4, COX6A1, COX8C, COX17, BCS1L, SMIM20, COX7A2, TACO1, COA1, COX411, COX6B1, PET100, NDUFA4L2, COX5B and COX20.

In aspects, the mtDNA gene is a complex V subunit and/or assembly factor selected from MT-ATP8, MT-ATP6, ATP23, ATP5PO, ATP5F1C, ATP5F1E, ATPAF2, ATP5PF, OXA1L, ATP5MJ, ATP23, TMEM242, ATP5MK, ATP5MC1, ATP5MGL, ATP5F1A, FMC1, PPIF, ATP5MF, TMEM70, ATP5F1B, ATP5F1D, ATP5PD, ATP5MC3, ATP5ME, ATP5MC2, ATP5MG, ATP5PB and ATPAF1.

In aspects, the mtDNA gene is a component of mitochondrial inner membrane selected from MT-ND1, MT-ND2, MT-CO1, MT-CO2, MT-ATP8, MT-ATP6, MT-CO3, MT-ND3, MT-ND4L, MT-ND4, MT-ND5, MT-ND6, MT-CYB, NDUFA6, SMDT1, TIMM22, SMDT1, RDH13, BDH1, RDH13, AGK, NDUFA3, SMDT1, RDH13, RDH13, ATP23, RDH13, MRPS18B, MRPS18B, MRPS18B, TAMM41, CHCHD10, RDH13, NEU4, TIMM22, RDH13, MRPS36, MRPL45, PAM16, RDH13, MRPS18B, RDH13, SURF1, COX8C, MRPS18B, SLC25A15, DNAJC15, MRPS18B, SLC25A26, MRPS6, NDUFS1, MRPS31, SLC25A6, SLC25A24, HCCS, MRPS12, OTC, SAMM50, SLC25A17, PLSCR3, ATP5PO, MRPL39, ATP5F1C, MRPL36, ATP5F1E, SLC25A37, SLC25A30, NDUFS3, PTPMT1, NDUFV3, NDUFV2, SLC25A16, ATPSCKMT, MRPS30, LYN, MRPS26, SLC25A29, COX412, BCL2L1, SLC25A18, MRPL23, SLC25A52, SLC25A47, UQCC2, CYP11A1, CDS2, TTC19, CKMT2, MRPL57, MICU2, GRPEL1, ROMO1, ATP5PF, DMAC1, COX7C, COX5A, MRPL54, AIFM1, NDUFS4, MRPS28, COX7B, OXA1L, ATAD3B, HADHA, UQCRB, CYP11A1, IMMP1L, TIMM8A, SFXN2, MTG1, DUSP21, MRPL16, SLC25A53, ADCK1, NDUFA13, MRPL13, NDUFAF5, NNT, MRPL15, HADHB, MICOS10-NBL1, ATP5MJ, SFXN4, CABS1, MTG2, COX7B2, FPGS, COX7A2L, SFXN5, ALAS2, SLC25A33, COQ4, SHMT2, SLC27A1, COA3, SLC9B2, UQCRH, UQCR10, MRPL1, MRPL19, MRPL41, MTFP1, MRPL50, NDUFAF4, PDE2A, ATP23, CYP11B2, MICOS10, MRPS9, TMEM242, NDUFA4, CPOX, SLC25A1, ABCB7, MRPS11, COX7A1, GCAT, DELE1, COX11, CPT2, ATP5MK, PTPN1, SRC, MRPL40, PRODH, TIMM21, OMA1, GRPEL2, AIFM3, MRPL46, ATP5MC1, NDUFA1, SCO2, MCUB, FECH, CYP11B1, TIMMDC1, TIMM9, MRPL22, CKMT1B, CKMT1A, TMEM177, RAB5IF, RPS3, UQCRFS1, SFXN1, PMPCA, APOO, MRPS23, TYMS, SLC25A31, AFG3L2, COQ7, SLC25A14, MRPS17, CYCS, ATP5MGL, SMDT1, CYP24A1, SLC25A42, TRMT10B, SLC25A22, SLC25A51, MPC1L, NDUFB4, TIMM50, COQ9, LETM2, PISD, CHCHD6, NDUFA8, SLC25A24, TMEM14C, NDUFB1, TIMM8B, SDHD, TMEM11, GATM, L2HGDH, ATP5MF-PTCD1, CYC1, SQOR, COX16, MRPL18, HSD3B2, MRPL23, ATP5F1A, MTX3, CHCHD10, MRPL2, LGALS3, DUSP18, PLSCR3, TOML2, NDUFS6, MRPS10, TRAP1, SLC25A4, SLC25A35, COX8A, MPC1, MRS2, PAM16, SURF1, IMMP2L, BOK, SLC25A12, SCO1, NDUFA2, GADD45GIP1, COX6B2, MPC2, APOOL, NDUFB6, STMP1, NDUFB9, UQCC1, MRPL52, COX10, CHCHD1, TIMM23, UQCRC2, COX18, MRPL4, SPNS1, CRLS1, NDUFB11, WDR93, MGARP, COX6C, NDUFC1, LETM1, ENDOG, MRPL14, MPV17L2, CRAT, NOA1, TIMM23B, MRPL12, SLC25A10, TMEM65, MTX2, DMAC2, CLPX, AGK, MRPS18B, MRPL38, BDH1, MRPL21, NDUFA7, CHCHD3, PPIF, MRPS14, SLC25A25, ALDH18A1, COA8, DMAC2L, SPHK2, HSPA9, NDUFAF1, NDUFB8, TIMM17B, GPD2, MRPS18A, SLC25A43, SLC25A5, SLC25A20, NDUFAF3, NDUFAF2, C15orf48, PLA2G4A, SLC25A13, MRPL53, TAFAZZIN, COX6A2, TIMM13, PARL, PTCD3, MRPS34, MRPL35, MRPL48, AMBP, NDUFB7, UCP3, NDUFAB1, TIMM44, FLVCR1, PLA2G4B, MRPL10, NDUFB2, MRPS33, CIBAR1, ATP5MF, COX6A1, SPG7, COQ5, COQ3, MRPL11, TIMM17A, MRPS7, SLC25A19, PTPMT1, COQ8B, SLC25A27, TIMM29, PINK1, TMEM70, LDHD, TIMM10, MRPL47, UCP2, DMD, DNAJC30, DAP3, COQ2, MRPS18C, CYP2E1, MICU1, MRPL32, MRPL34, MRPS12, UCP1, COQ6, PMPCB, MRPS35, MICU3, SLC25A32, YME1L1, ETFDH, TMEM126B, TMEM126A, UQCRQ, CYP2U1, LDHB, NDUFC2-KCTD14, NDUFA5, NDUFC2, MRPL17, SDHA, MCU, CYP1A1, GUF1, MRPS2, COX8C, SLC25A21, SLC25A2, SLC25A41, SLC25A23, MRPL30, PPOX, CLU, ABCB8, MRPS16, HSPD1, ATP5F1B, GHITM, NDUFA3, NDUFA10, UQCC3, ACAD11, MRPL43, TIMM22, MRPL51, NDUFAF6, TOMM40, SDHB, PSEN1, HSD3B1, HIGD2A, MRPL27, SLC41A3, OPA1, SLC25A40, SLC25A39, NDUFV1, PHB2, TMEM186, MRPL28, NME4, TIMM10B, BCS1L, ERAL1, DHRS13, ATP5F1D, CYP27A1, FDXR, MRPL58, ATP5PD, MRPS36, ATP5MC3, RCC1L, ACADVL, NDUFB5, MRPS27, MRPL42, DHODH, ACAD9, MRPL20, ABCB10, DHFR2, LETMD1, SFXN3, SLC25A48, SMIM20, COX15, HIGD1A, MRPS24, MCUR1, C12orf73, LRRK2, MICOS13, ATP5ME, CCDC51, SLC25A28, NDUFA11, UQCRC1, MRPL55, MTHFD2L, RSAD2, SLC8B1, COQ10A, COQ8A, NDUFS5, CNP, SDHC, NDUFS2, NDUFB10, SIRT4, NDUFA12, CHDH, SLC25A3, NDUFS7, COX7A2, SLC22A14, MTLN, NDUFB3, ECSIT, FGR, IFI6, COA1, MRPL37, ATP5MC2, PSEN2, SLC25A11, COX411, MRPL24, COX6B1, PHB1, MRPS22, NEU4, MRPL3, RDH13, UQCR11, PGS1, PRODH2, PET100, FOXRED1, SLC25A26, IMMT, NDUFA4L2, MTX1, MRPL9, THEM4, NDUFA9, CPS1, ATP5MG, TAMM41, EFHD1, MYOC, ATAD3A, MRPL49, EXOG, COQ10B, SLC25A45, SLC25A38, SLC25A36, MPV17, NDUFS8, MRPS15, COX5B, MRPS25, DNAJC19, MAIP1, MRPL33, DNAJC11, MRPS5, ATP5PB, MRPL44, SLC25A34, UQCRHL, COX20, MRPS21 and AURKAIP1.

In aspects, the mtDNA gene is an integral component of the mitochondrial inner membrane selected from MT-CYB, SMDT1, SMDT1, AGK, SMDT1, CHCHD10, SAMM50, OXA1L, SFXN2, MICOS10-NBL1, SFXN4, SFXN5, COA3, MICOS10, COX11, SCO2, MCUB, TMEM177, SFXN1, APOO, AFG3L2, SMDT1, MPC1L, PISD, CHCHD6, TMEM11, L2HGDH, COX16, MTX3, CHCHD10, MPC1, SCO1, MPC2, APOOL, TIMM23, COX18, TIMM23B, MTX2, AGK, CHCHD3, HSPA9, TIMM17B, SPG7, TIMM17A, SLC25A19, COQ2, ETFDH, MCU, GHITM, UQCC3, SFXN3, MCUR1, MICOS13, CCDC51, SLC25A3, SLC22A14, MTLN, COA1, PET100, IMMT, MTX1 and DNAJC11.

In aspects, the mtDNA gene is a matrix protein selected from SMDT1, SMDT1, BDH1, SSBP1, SMDT1, MRPS18B, MRPS18B, MRPS18B, MRPS36, MRPL45, PAM16, DNAJA3, MRPS18B, MRM1, HSD17B8, HSD17B8, HSD17B8, MRPS18B, MIPEP, DNAJC15, MRPS18B, HSD17B8, ACSS2, HSD17B8, MRPS6, NDUFS1, MCAT, MRPS31, SARS2, MRPS12, OTC, PCCA, IDH3B, ACSS1, MRPL39, AK3, NUBPL, ATP5F1C, MRPL36, FASTKD3, TOP3A, PABPC5, PIN4, ATP5F1E, SUCLA2, NDUFS3, ISCA2, PCK2, CARS2, MRPS30, RAD51, NAXD, MRPS26, ME2, LDHAL6B, PDSS1, BCL2L1, GLUD2, MRPL23, OXCT1, TFAM, GLRX5, UQCC2, MMUT, CYP11A1, DHTKD1, TMLHE, TEFM, GLUD2, MRPL57, GRPEL1, NFS1, SDHAF4, HOGA1, PDP1, MRPL54, GLYAT, MRPS28, SOD1, OXA1L, DMGDH, ISCA1, MTRES1, RIDA, TXNRD2, HADHA, DNA2, CYP11A1, OAT, MTG1, DUSP21, METTL15, MRPL16, TOP3A, ALDH4A1, DLST, ETFBKMT, MRPL13, MRPL15, HADHB, DHRS2, MTG2, MPST, PRDX3, FPGS, TTC5, ACADSB, CA5B, ALAS2, TRUB2, NUDT9, VDAC1, SHMT2, GLS2, PNPT1, PDE12, HYKK, MRPL1, LACTB2, MRPL19, MRPL41, MRPL50, TOP1MT, GLDC, PDE2A, MRPS9, PDSS2, PPA2, FDX1, PCK2, ETNPPL, ACSS3, AUH, MRPS11, ECI1, FOXO3, PARS2, ACO2, PTPN1, MRPL40, PRODH, GRPEL2, SUCLG1, MALSU1, ABHD10, MRPL46, BCKDHB, FTMT, SCO2, NR3C1, MTRF1L, AK4, FECH, ALDH1L2, MRPL22, TBRG4, ALDH7A1, PDHA2, METTL4, FXN, ERBB4, RPS3, IDH3A, METTL17, ACOT9, PMPCA, MRPS23, PRDX5, TYMS, GPT2, SDHAF3, MTERF1, POLDIP2, ARG2, ARL2, MRPS17, FARS2, ALDH2, SMDT1, CBR4, FASTKD5, TXN2, ALDH5A1, ACOT13, HSD17B10, DGLUCY, ACADL, MRRF, PPM1K, PDP2, NT5M, BCO2, SOD2, MTERF2, MRPL18, MRPL23, ATP5F1A, POLG, MRPL2, AADAT, FASTKD2, ACAA2, MRPS10, SARS2, TFB1M, BCAT2, TRAP1, LYRM7, PAM16, MTERF4, TST, DNAJA3, GADD45GIP1, HARS2, IDH3G, HIBADH, LYRM4, ELAC2, BCKDK, MRPL52, CASQ1, GSR, GSTZ1, CHCHD1, SUPV3L1, MRPL4, TUFM, PITRM1, BCKDHA, SSBP1, MRPL14, ACAT1, REXO2, MPV17L2, TFB2M, AKR1B15, PDK3, MRPL12, HSPA1L, MTHFS, D2HGDH, CLPX, NMNAT3, LIAS, MRPS18B, MRPL38, BDH1, GCSH, MRPL21, NDUFA7, PPIF, MRPS14, PDPR, HSPA9, OGDHL, NUDT2, NDUFAF1, NDUFB8, TWNK, MTHFD1L, MRPS18A, KARS1, GLS, SLC25A5, ECHS1, PDHA1, DDX28, NADK2, TSFM, PARG, NDUFAF7, RNASEL, MLYCD, SIRT5, PTCD1, MRPL53, ACAD10, PTCD3, MRPS34, MRPL35, MRPL48, HAGH, GARS1, EARS2, NDUFAB1, TIMM44, SARDH, GLUD1, MRPL10, MRPS33, POLG2, COQ5, PRIMPOL, PDK1, DIMT1, COQ3, MRPL11, NAT8L, MRPS7, ATXN3, LIPT2, ALDH1B1, COASY, MRPL47, ATG4D, CCAR2, IDH2, DAP3, MRPS18C, ARL2BP, HSD17B8, MRPL32, MRPL34, MRPS12, RPUSD4, SUCLG2, ACSF3, NAGS, MMAA, MCCC2, PMPCB, ALDH6A1, MRPS35, ETFDH, SIRT3, PRORP, ISCU, IVD, AMT, PYCR1, EXD2, ACOT2, CDKN2A, MRPL17, DECR1, VDAC2, MRM3, HADH, GUF1, MRPS2, MMAB, CLPP, ALKBH7, LIPT1, MRPL30, ALAS1, C2orf69, NUDT13, HSPE1, MRPS16, HSPD1, QARS1, GFM2, SDHAF2, TK2, ATP5F1B, ADHFE1, NDUFA10, AASS, MYG1, DLAT, MRPL43, MRM2, MRPL51, TP53AIP1, GSTK1, FASTKD1, TDRD7, ACSM5, ACSM2A, GCDH, NUDT1, CCNB1, GOT2, PDK2, HMGCS2, ARHGAP11B, MRPL27, TRMT5, LRRC59, PHYKPL, ACSM2B, ACSM3, ETFA, ACSM1, ABAT, MTHFD2, ACP6, MRPL28, NME4, ERAL1, ATP5F1D, CYP27A1, FDXR, WARS2, MRPL58, GLRX2, GPX1, MRPS36, PDHB, GRSF1, ACADVL, HTD2, ACSM6, MRPS27, MRPL42, PYROXD2, MRPL20, DHFR2, NSUN3, AGXT2, ETFB, RARS2, PUS1, STYXL1, MDH2, MRPS24, CDK1, LRRK2, FASTK, LONP1, PPTC7, MRPL55, MTHFD2L, IBA57, TERT, SUOX, PDHX, DLD, OGG1, OGDH, MECR, DARS2, CS, DHX30, YARS2, NDUFS2, LARS2, SIRT4, NDUFS7, MPG, DGUOK, ACOT11, PDK4, POLRMT, SDHAF1, ABCE1, LRPPRC, MRPL37, ETHE1, NAXE, MRPL24, ACADS, MRPS22, MRPL3, DBT, MCCC1, ACAD8, FAHD1, ACSM4, BLOC1S1, CASA, MRM1, TP53, TRMT10C, FDX2, MRPL9, THEM5, THEM4, NDUFA9, CPS1, NARS2, ACSF2, RPUSD3, FH, ATAD3A, MRPL49, NSUN4, PC, ADPRS, NDUFS8, MRPS15, C1QBP, SHC1, ME3, FLAD1, GFM1, TRNT1, BTD, MRPS25, DNAJC19, CHPF, MAIP1, PYCR2, MCEE, TARS2, MARS2, PCCB, HMGCL, HIBCH, IARS2, MRPL33, TRMT61B, MRPS5, ATP5PB, MRPL44, OXCT2, TRIT1, MRPS21, AURKAIP1, ACADM and PARK7.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

### Example 1

Re-engineering the 13 mtDNA Encoded Proteins for Nuclear Expression

FIG. 1B depicts gene constructs that were used in this study. Each construct utilized the same promoter (pCMV) with different elements. Levels of expression of OND1 were compared as described below.

First, mRNA expression levels were determined for each construct. FIG. 1C is a bar graph showing the levels of oxidative phosphorylation (OxPhos) protein oND1 mRNA expression in ND1 null cell lines for each construct (relative to GAPDH). The highest level of expression was found in the PUM construct. Similarly, FIG. 1D is a bar graph showing the levels of OND1 mRNA expression in ND1 null cell lines for each construct (relative to COX10). The PUM construct also had the highest level of expression.

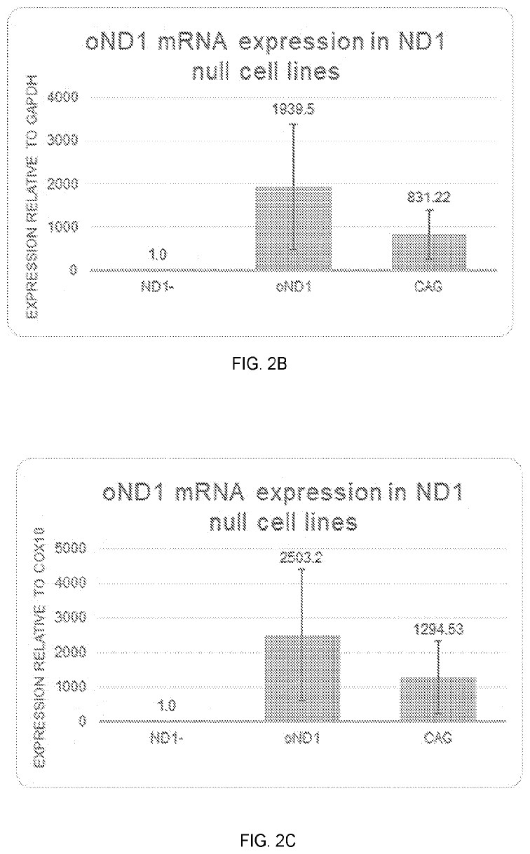

Next, levels of expression were compared using different promoters (i.e., pCMV and pCAG). FIG. 2A depicts pCMV and pCAG constructs. FIG. 2B is a bar graph showing the levels of OND1 mRNA expression in ND1 null cell lines for each construct (relative to GAPDH). FIG. 2C is a bar graph showing the levels of oND1 mRNA expression in ND1 null cell lines for each construct (relative to COX10). In both studies, the pCMV promoter demonstrated higher levels of OND1 expression.

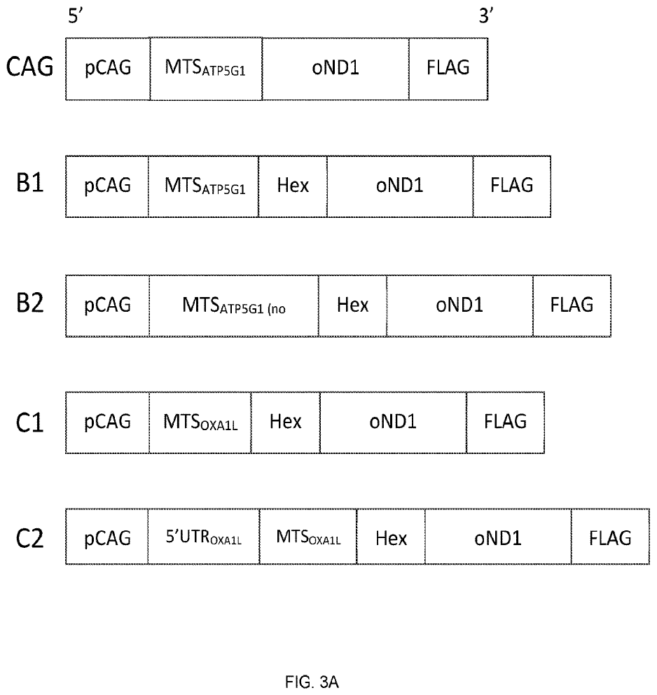

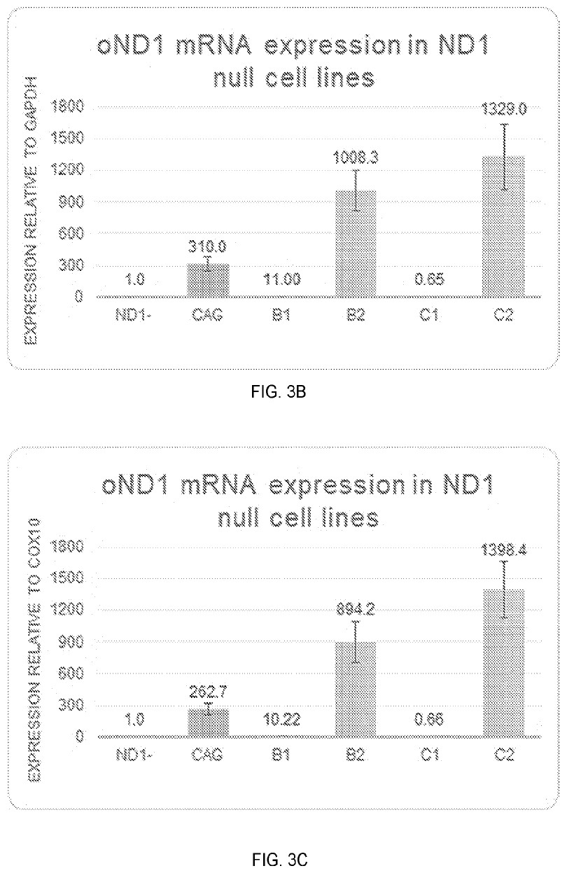

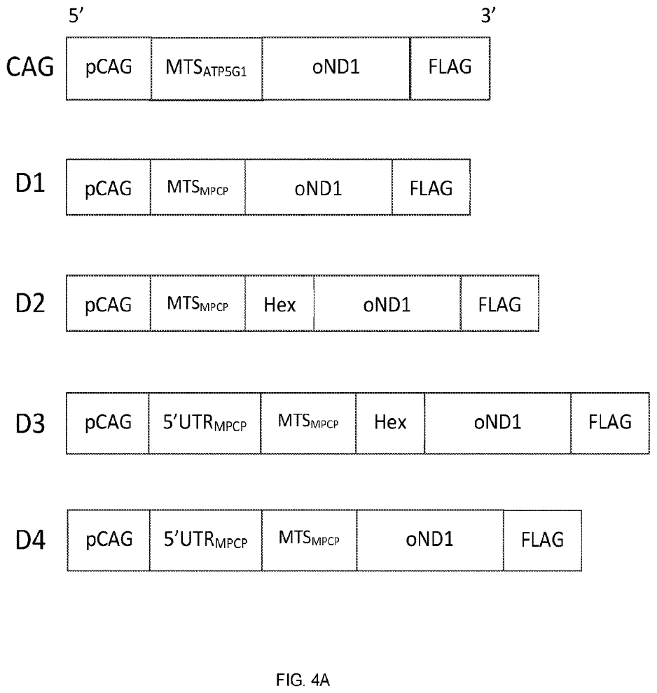

In the next study, levels of expression were compared using different N-terminal export signals (i.e., Hexapeptide and OXA1L). FIG. 3A depicts constructs that were compared.

FIG. 3B is a bar graph showing the levels of OND1 mRNA expression in ND1 null cell lines for each construct (relative to GAPDH). FIG. 3C is a bar graph showing the levels of oND1 mRNA expression in ND1 null cell lines for each construct (relative to COX10). The C2 (pCAG, OXA1L 5′UTR, OXA1L MTS, Hex) construct demonstrated the highest expression.

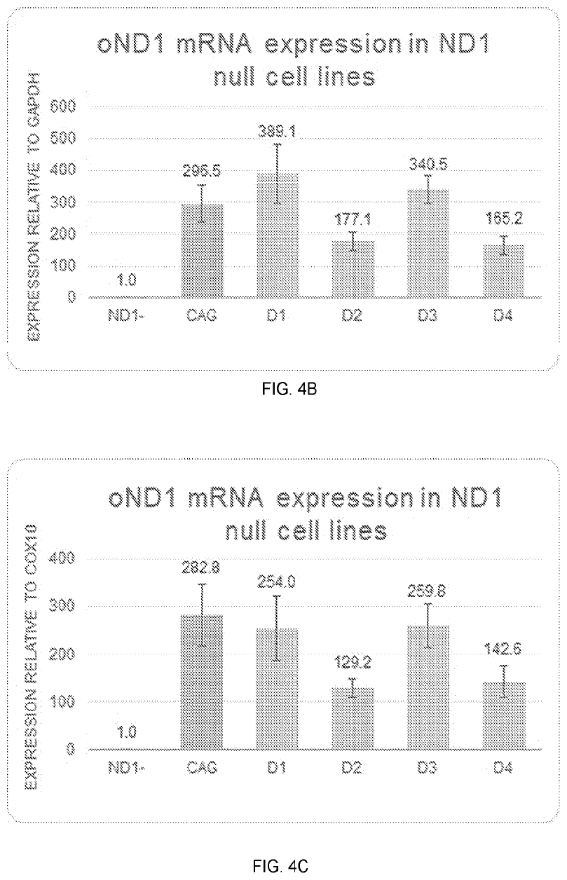

The pre-sequence of MPCP can constitute a signal which can be recognized by the import and assembly machinery as being destined for the mitochondrial inner membrane (MIM). FIG. 4A depicts constructs with MPCP sequences. FIG. 4B is a bar graph showing the levels of OND1 mRNA expression in ND1 null cell lines for each construct (relative to GAPDH). FIG. 4C is a bar graph showing the levels of OND1 mRNA expression in ND1 null cell lines for each construct (relative to COX10). The D1 construct and CAG construct demonstrated the highest levels of expression respectively.

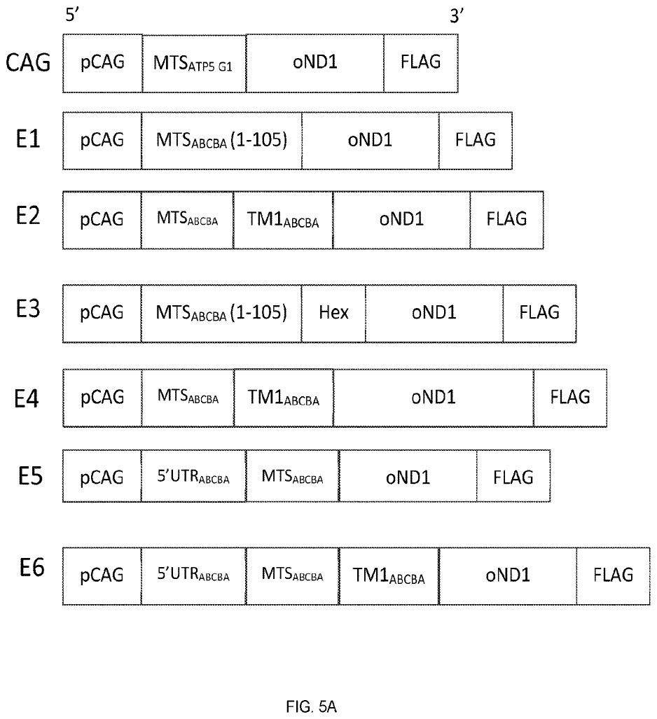

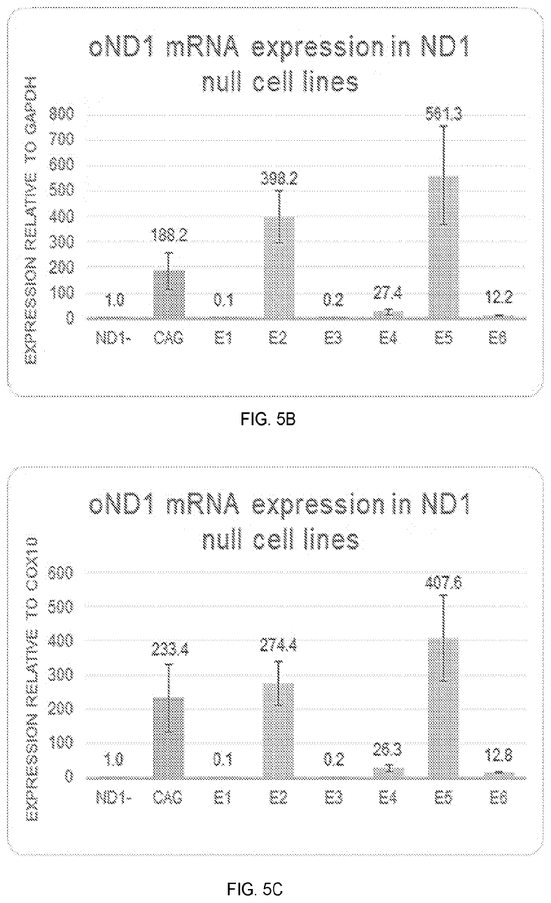

ABCBA is a mitochondrial ABC transporter of the inner membrane and has an N-terminal mitochondrial targeting sequence (MTS). FIG. 5A depicts constructs that include ABCBA sequences. FIG. 5B is a bar graph showing the levels of OND1 mRNA expression in ND1 null cell lines for each construct (relative to GAPDH). FIG. 5C is a bar graph showing the levels of OND1 mRNA expression in ND1 null cell lines for each construct (relative to COX10). The E5 construct demonstrated the highest levels of expression.

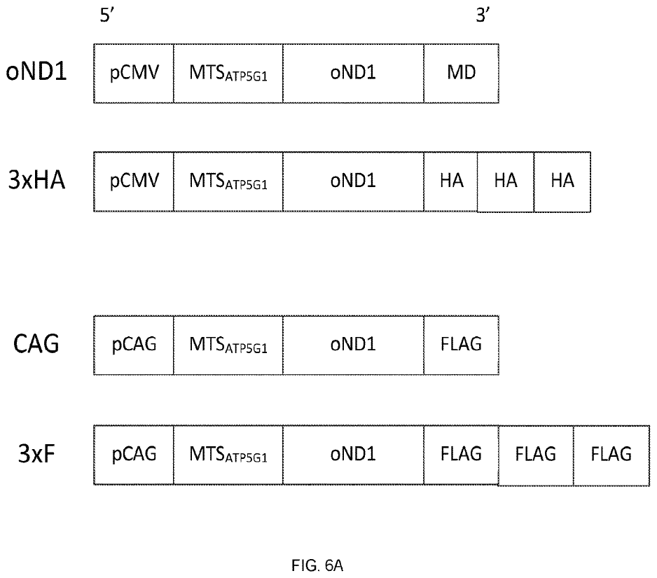

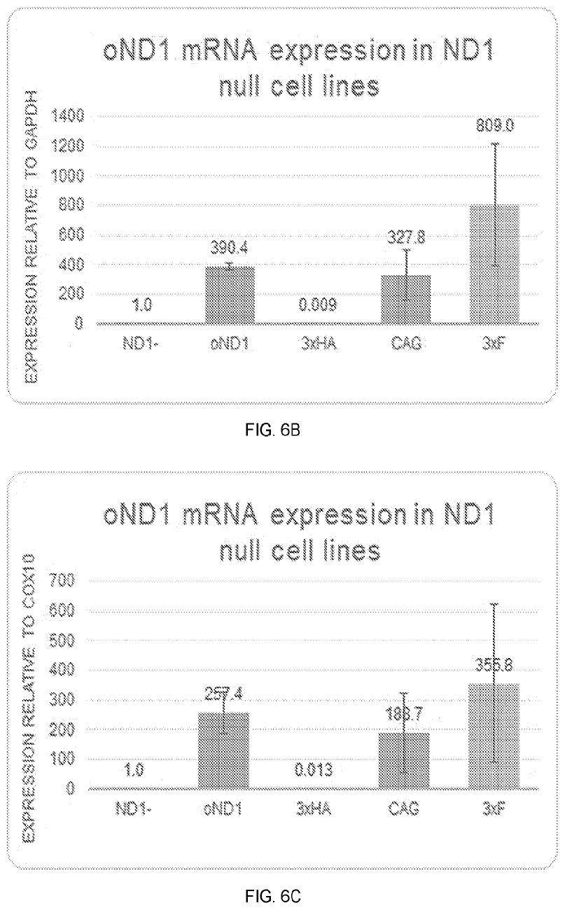

FIG. 6A depicts constructs with triple protein tags according to embodiments of the invention. FIG. 6B is a bar graph showing the levels of OND1 mRNA expression in ND1 null cell lines for each construct (relative to GAPDH). FIG. 6C is a bar graph showing the levels of OND1 mRNA expression in ND1 null cell lines for each construct (relative to COX10). The 3×F construct demonstrated the highest levels of expression.

FIG. 7A is graphical depiction of mesohydrophobicity versus local hydrophobicity of transmembrane region 1 of ATP6. Similarly, FIG. 7B is graphical depiction of mesohydrophobicity versus local hydrophobicity of transmembrane region 1 of ATP6.

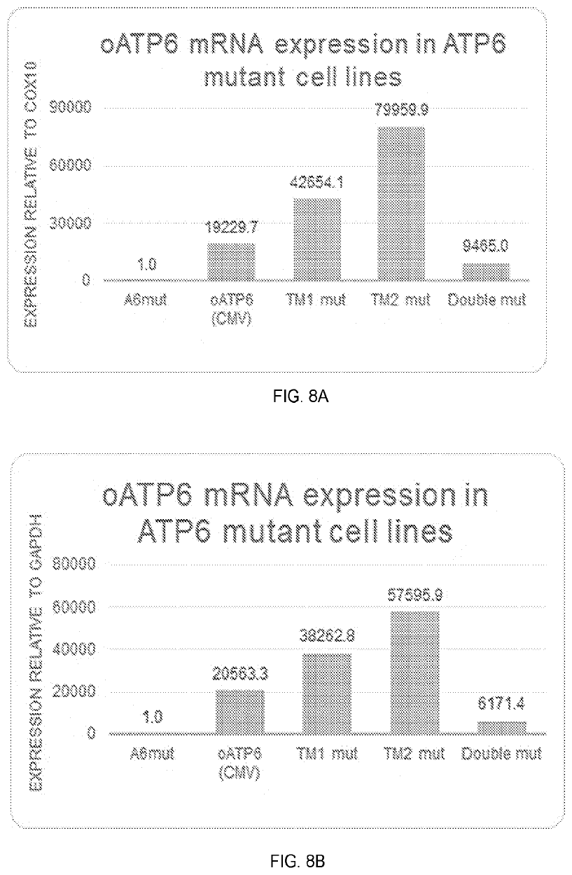

FIG. 8A is a bar graph showing the levels of oATP6 mRNA expression in ATP6 mutant cell lines for different constructs of COX10. FIG. 8B is a bar graph showing the levels of oATP6 mRNA expression in ATP6 mutant cell lines for different constructs to GAPDH. The TM2 mutant cell line demonstrated the highest levels of expression.

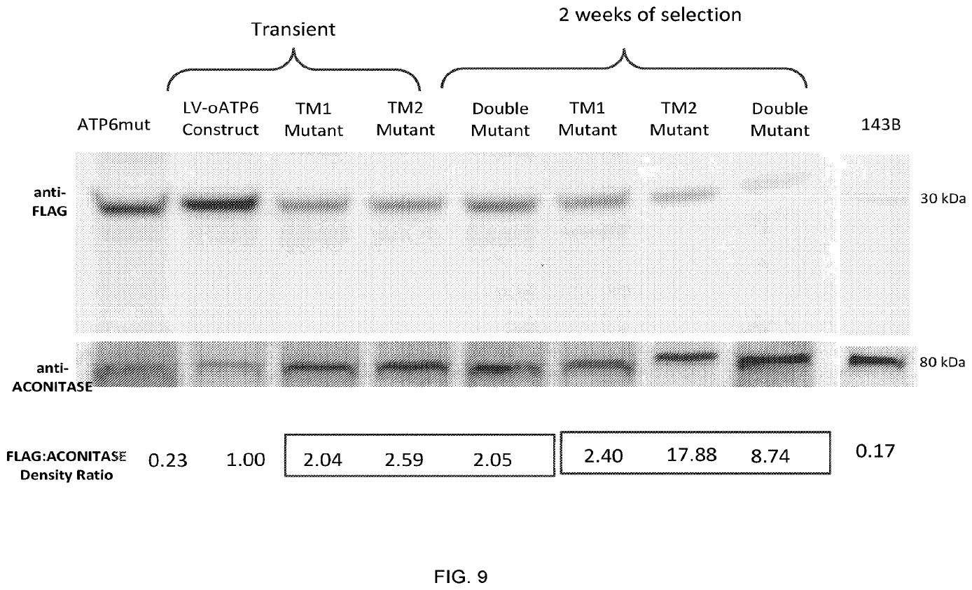

FIG. 9 is an image of SDS PAGE of mitochondrial fractions with a comparison of anti-FLAG and anti-ACONITASE expression. The TM2 Mutant demonstrated the highest flag:aconitase density ratio (i.e. 17.88).

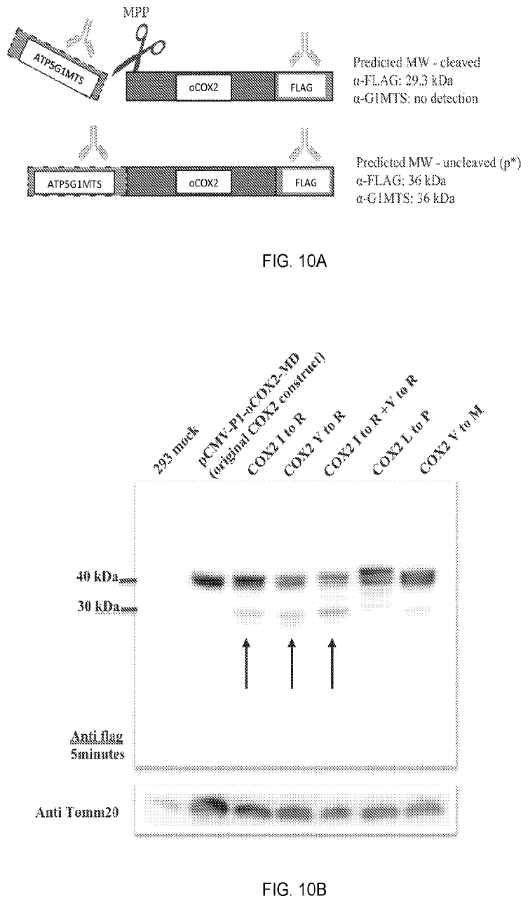

FIG. 10A depicts the cleaved and uncleaved COX2 protein. The predicted molecular weight and marker detection for the cleaved and uncleaved correspond to the molecular weights demonstrated as kDa in the SDS PAGE gel.

FIG. 10B is an SDS PAGE showing stable expression in COX2 site-directed mutagenesis constructs. It shows the influence of reducing transmembrane hydrophobicity on the import of COX2. The purified mitochondrial fractions were resolved on 4-12% SDS PAGE gels and probed for Anti FLAG and Anti TOMM 20 (an outer mitochondrial membrane protein marker). The lanes represent the (1) 293 mock negative control (2) original COX2 construct (3) 131R mutagenesis construct (4) Y40R mutagenesis construct (5) 131R+Y40R mutagenesis construct (6) L74P mutagenesis construct and (7) V75M mutagenesis construct respectively.

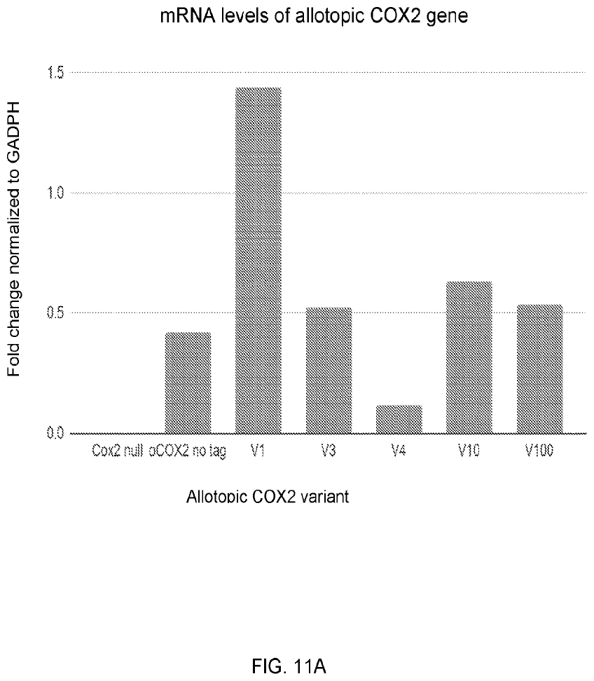

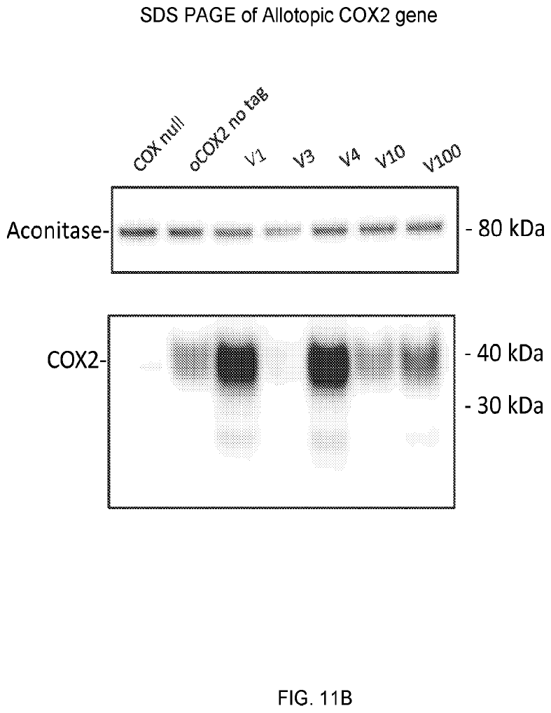

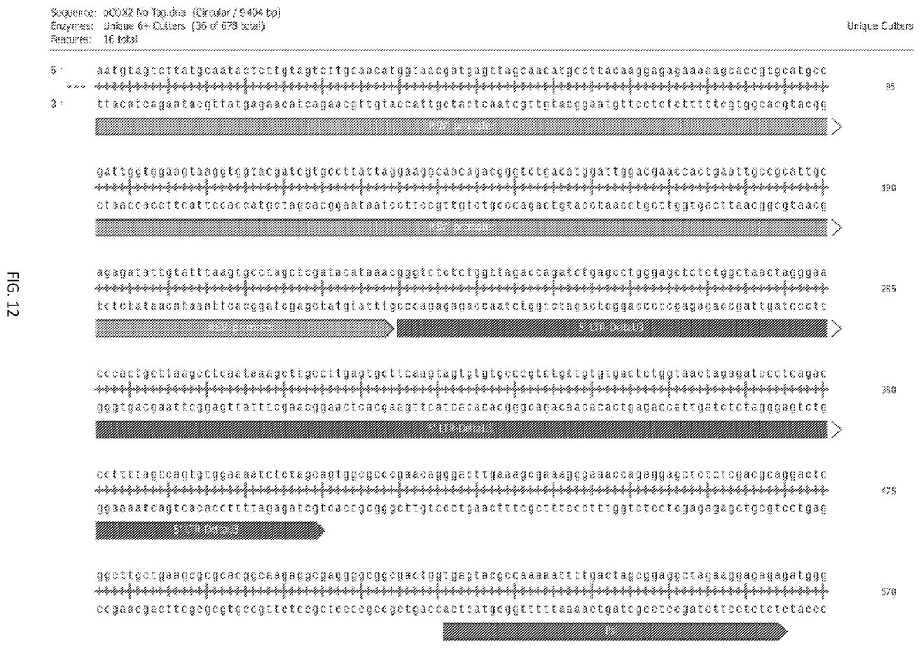

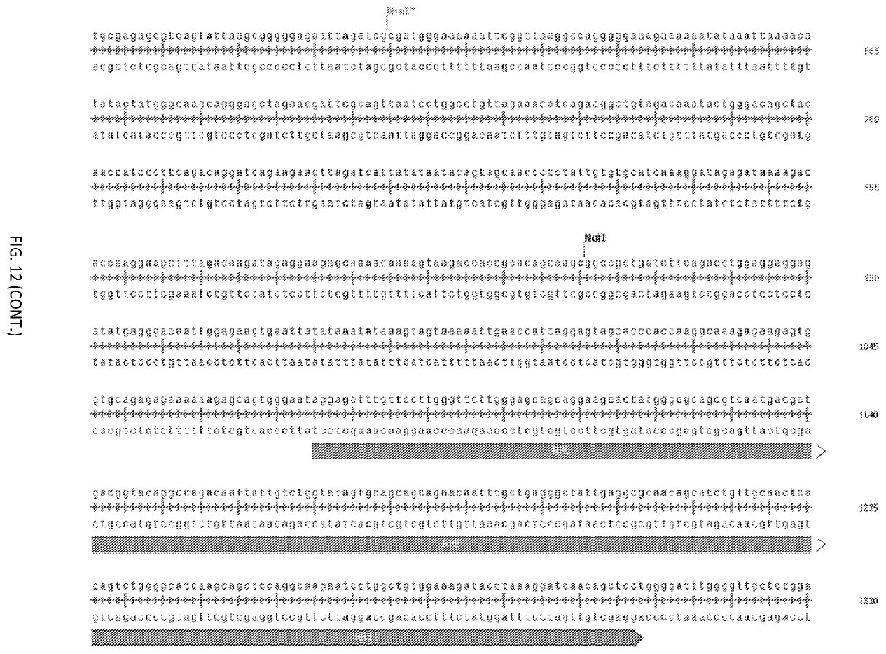

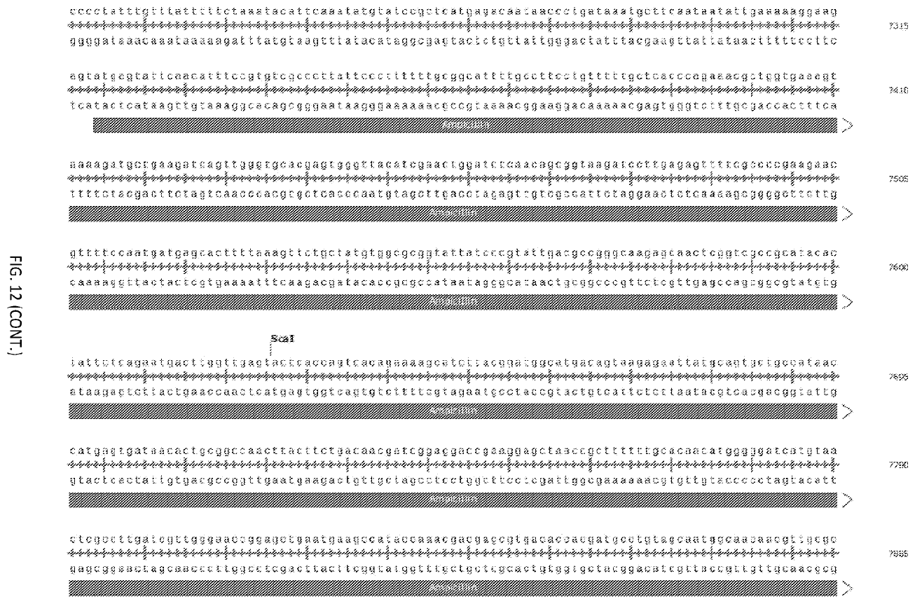

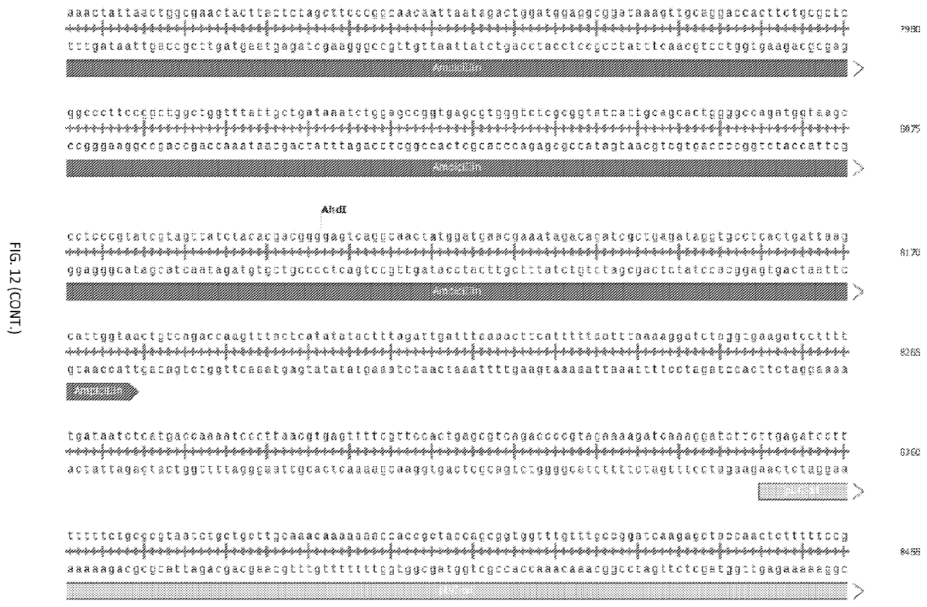

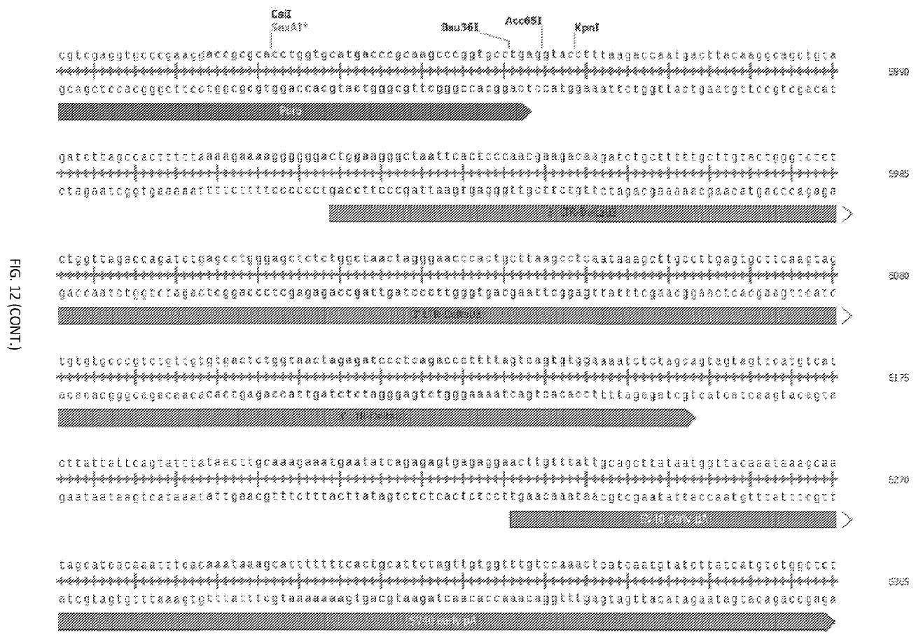

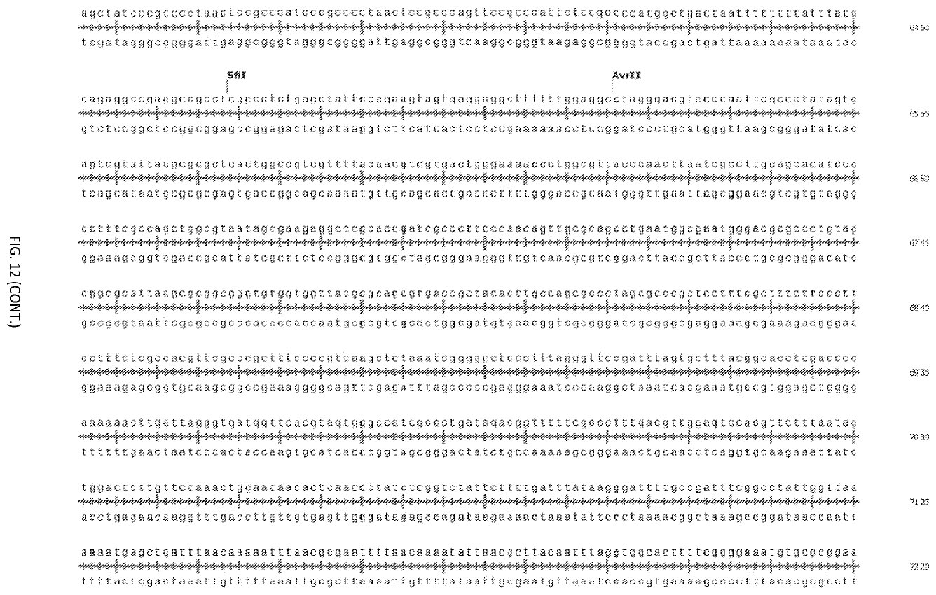

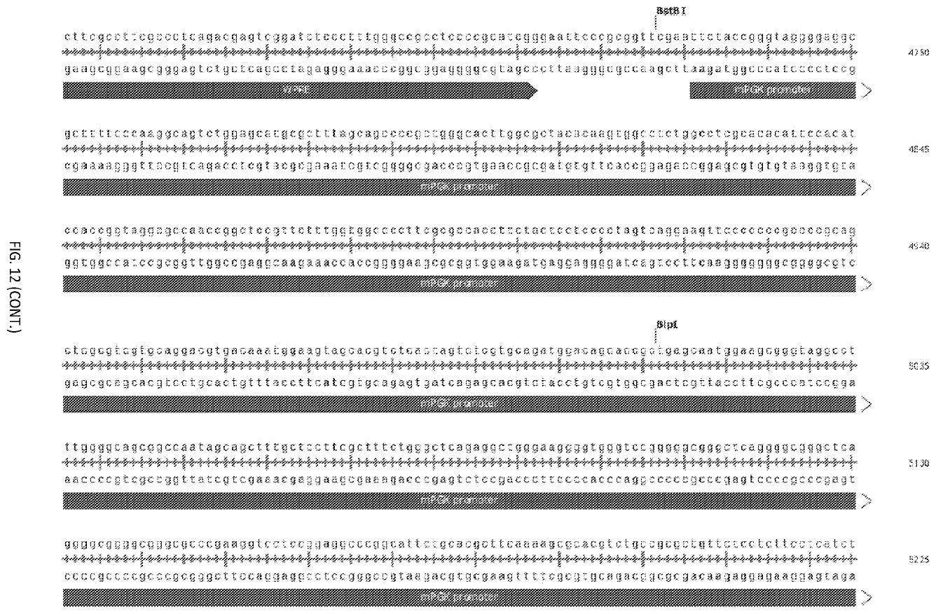

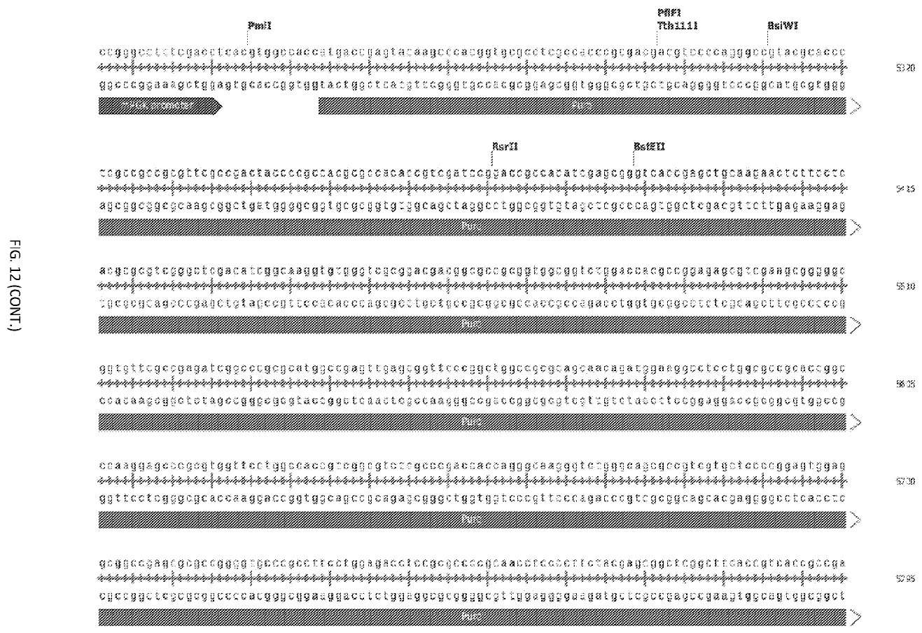

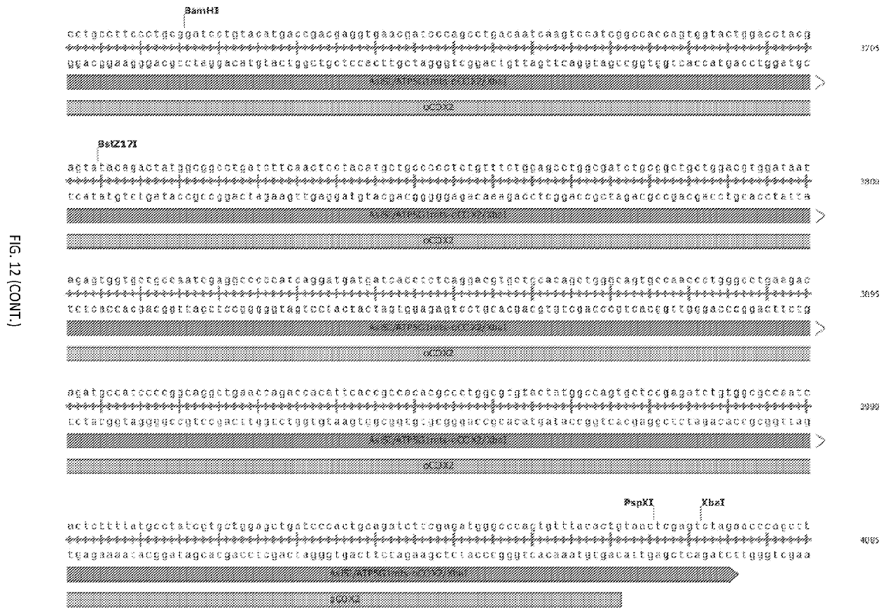

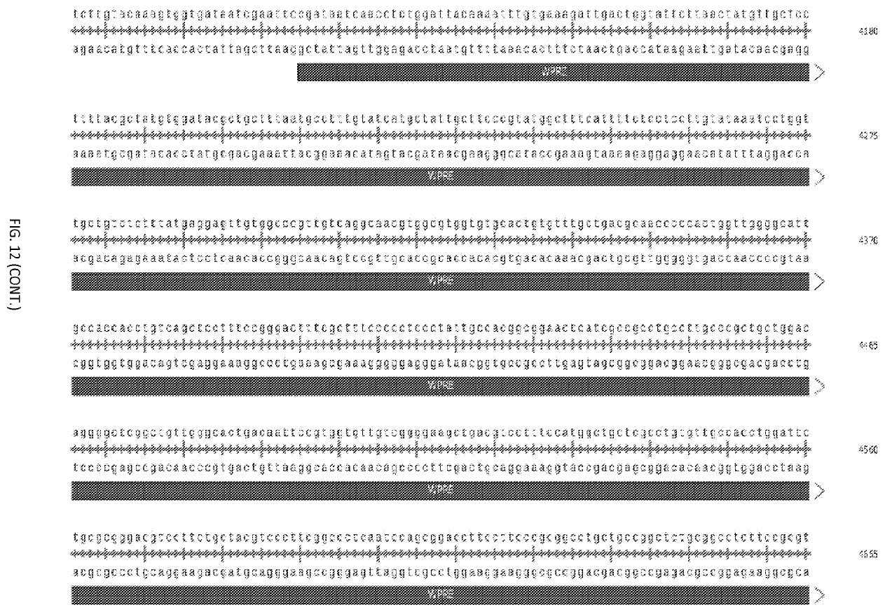

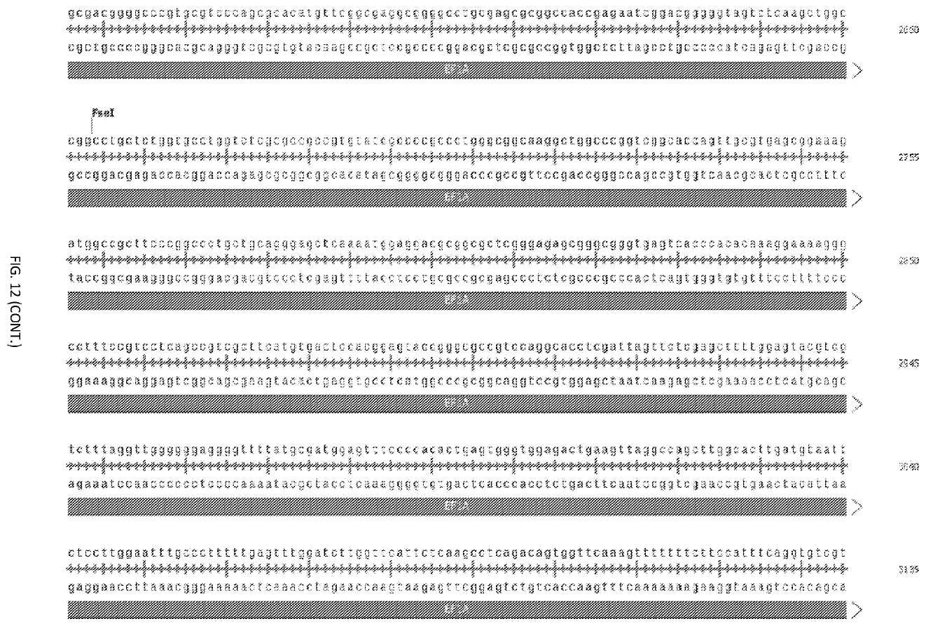

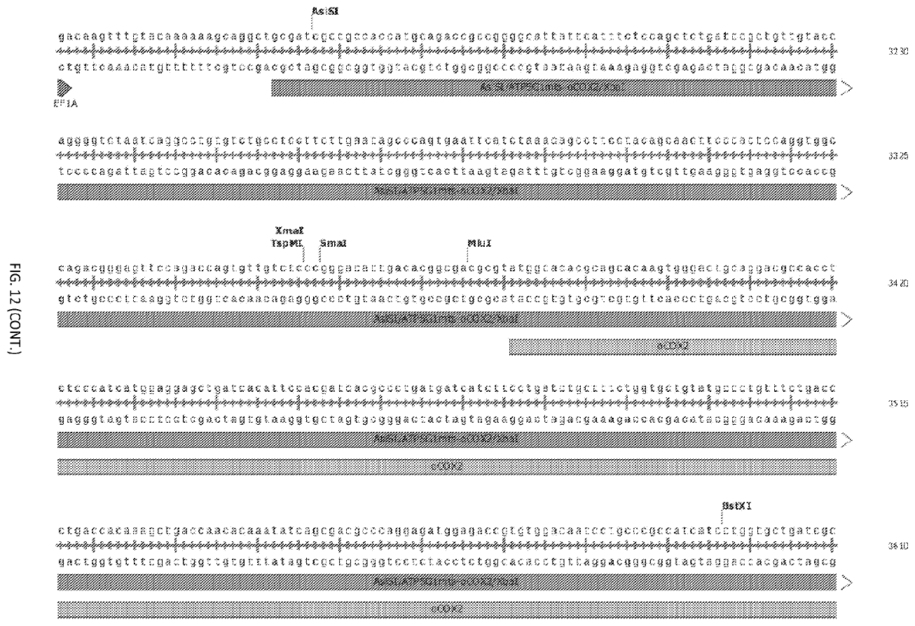

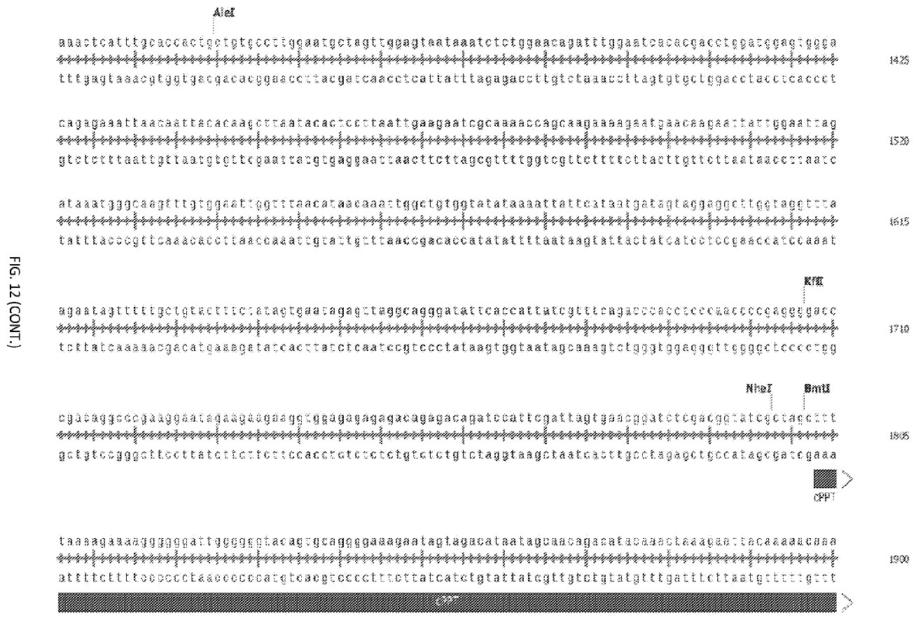

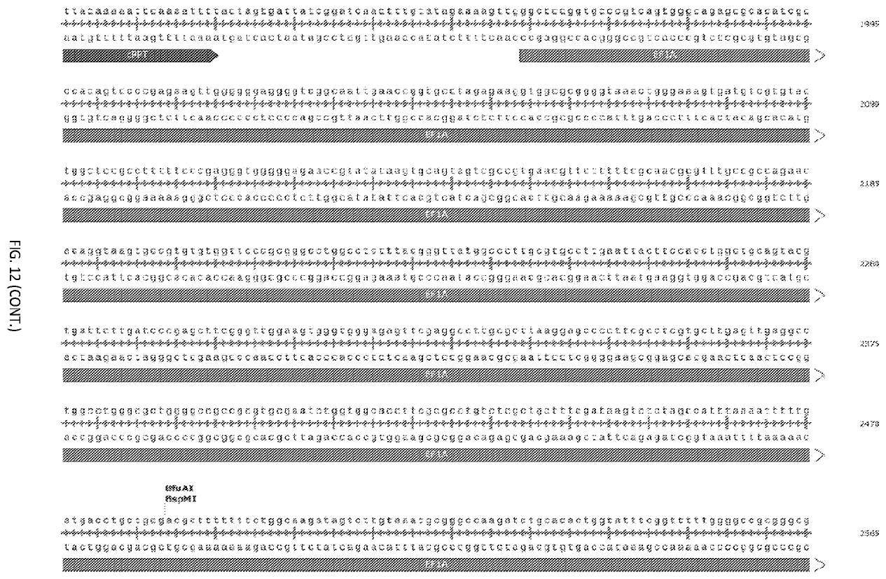

FIG. 11A shows mRNA levels for different versions of codon-optimized allotopic COX2 gene in comparison to GAPDH. V1 showed the highest expression of mRNA while V4 was lowest.

Similarly, FIG. 11B shows SDS PAGE profiles for different versions of codon-optimized allotopic COX2 in a COX2 null cell line upon stable selection. V1 and V4 demonstrated the highest level of protein expression.

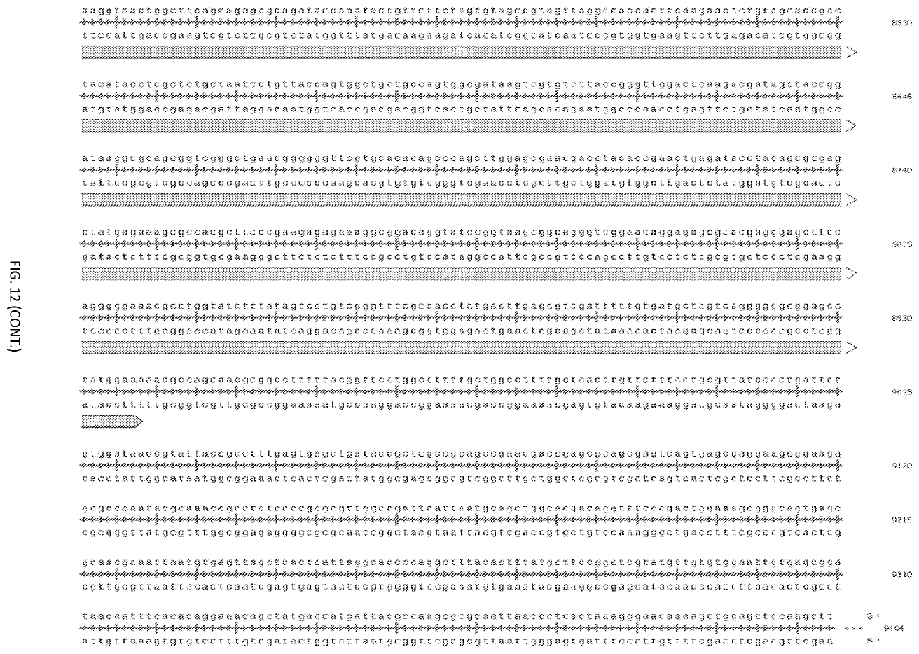

FIG. 12 shows a plasmid sequence of a COX2 variant according to embodiments.

### Example 2

### Treatment of Leigh Syndrome Via Allotopic Expression

Mitochondrial DNA (mtDNA) in humans is a 16,569 base pair double-stranded circular DNA that encodes 13 vital proteins of the electron transport chain. Mitochondria occupy a central position in the overall metabolism of eukaryotic cells. Oxidative phosphorylation (OXPHOS), the Krebs's cycle, the urea cycle, the heme biosynthesis and the fatty acid oxidation take place within the organelle. Recently, another major role for mitochondria in determining the cellular life span was established, as they are recognized to be a major early mediator in the apoptotic cascade. Mitochondria are also a major producer of reactive oxygen species (ROS) causing oxidative stress and therefore inducers of cell death.

Defects in mitochondrial function are implicated in over 120 diseases. Mitochondrial pathologies are considered among the most common genetically determined diseases and are a health concern because they lack effective treatments or therapies. Mitochondrion is assembled with proteins encoded by genes distributed between mitochondrial and nuclear genomes. These genes include those encoding the structural proteins of the respiratory chain complexes I-V, their associated substrates and products, the proteins necessary for mitochondrial biogenesis, the apparatus to import cytoplasmically synthesized precursors and the proteins necessary for mitochondrial assembly and turnover.

Approximately half of human mitochondrial disorders are caused by pathogenic point mutations of mtDNA, one-third of which are located in coding genes. Mitochondrial DNA (mtDNA) disorders include Leigh syndrome, leukodystrophy w/complex II deficiency, cardiomyopathy & encephalopathy (complex I deficiency), optic atrophy and ataxia (complex II deficiency), hypokalemia and lactic acidosis, hepatopathy & ketoacidosis, hypertrophic cardiomyopathy, liver failure, renal tubulopathy (w/complex III deficiency) and encephalopathy (w/complex V deficiency), autosomal progressive external ophthalmoplegia, mitochondrial neurogastrointestinal encephalomyopathy, Alpers-Huttenlocher syndrome, ataxia neuropathy syndromes, infantile myopathy/spinal muscular atrophy and hypotonia.

In this example, a pediatric patient is diagnosed with Leigh syndrome (early onset), a genetic condition that affects the central nervous system. The infant appeared healthy at birth but gradually presented symptoms as cells in the nervous system began to break down or degenerate. Symptoms included feeding problems, seizures and continuous crying.

A pediatrician conducts a genetic analysis to identify the gene responsible for the disease in the patient: MT-ATP6 gene from making ATP. This is the most common mtDNA change in Leigh syndrome and prevents the MT-ATP6 gene from making ATP. In this example, allotopic expression is used to introduce in the nucleus a wild-type (i.e., healthy) copy of the gene mutated in the mitochondrial genome and import normal copies of the gene product into mitochondria from the cytosol. The gene is introduced into the patient via allotopic expression using the methods described herein.

An expression vector is produced in the form of a recombinant vector. The vector includes: a nucleic acid sequence encoding a mitochondrion-targeting signal (i.e., an MTS sequence), a sequence that encodes the protein to be delivered and a 3′ nucleic acid sequence. Additional codon sequences are introduced into the vector to (a) reduce hydrophobicity of the gene product, (b) reduce a localized charge of the gene product and (c) cause ribosome stalling. The vector is introduced into the patient. The vector can be, for example, a plasmid, or a virus, such as an integrating viral vector (e.g., a retrovirus, an adeno-associated virus (AAV), or a lentivirus) or a non-integrating viral vector, such as an adenovirus, an alphavirus, a Herpes Simplex Virus (HSV).

Within 48 hours, the patient's condition gradually improve. Approximately one month after treatment, the patient has no signs/symptoms of the disease. The physician conducts regular testing of MT-ATP6 gene expression and activity.

### Example 3

### Treatment of Apical Hypertrophic Cardiomyopathy and Neuropathy Via Allotopic Expression

In this example, a 17-year-old male patient visits a healthcare provider and presents signs/symptoms of apical hypertrophic cardiomyopathy and neuropathy due to a mitochondrial disorder. The provider measures mitochondrial energy-generating system (MEGS) capacity in the muscle along with enzyme analysis in muscle and fibrobasts. Thereafter, relevant portions of the patient's mitochondrial DNA were analyzed by sequencing. A homoplasmic nonsense mutation m.8529G→A (p.Trp55X) was found in the mitochondrial ATP8 gene in the patient's fibroblasts and muscle tissue. Reduced complex V activity was measured in the patient's fibroblasts and muscle tissue and was confirmed in cybrid clones containing patient-derived mitochondrial DNA.

Allotopic expression is used to introduce in the nucleus a wild-type (i.e., healthy) copy of the gene mutated in the mitochondrial genome and import normal copies of the gene product into mitochondria from the cytosol. The gene is introduced into the patient via allotopic expression using the methods described herein.

An expression vector is produced in the form of a recombinant vector. The vector includes: a nucleic acid sequence encoding a mitochondrion-targeting signal (i.e., an MTS sequence), a sequence that encodes the protein to be delivered and a 3′ nucleic acid sequence. Additional codon sequences are introduced into the vector to (a) reduce hydrophobicity of the gene product, (b) reduce a localized charge of the gene product and (c) cause ribosome stalling. The vector is introduced into the patient.

The vector can be, for example, a plasmid, or a virus, such as an integrating viral vector (e.g., a retrovirus, an adeno-associated virus (AAV), or a lentivirus) or a non-integrating viral vector, such as an adenovirus, an alphavirus, a Herpes Simplex Virus (HSV). In this example, the vector is introduced as a plasmid via electroporation.

Within 48 hours, the patient's condition gradually improves. Approximately one month after treatment, the patient has no signs/symptoms of the disease. The physician conducts regular testing of MT-ATP6 gene expression and activity.

### Example 4

### Treatment of Leber Hereditary Optic Neuropathy (LHON)

Leber hereditary optic neuropathy (LHON), or sudden vision loss, is an inherited form of vision loss. It often starts with a painless clouding or blurring in one or both eyes, and then worsens with a loss of sharpness and loss of color vision. LHON affects the central vision needed for detailed tasks such as reading, driving and recognizing faces. LHON can lead to one becoming legally blind.

LHON is a mitochondrial disease caused by a mutation in the mitochondrial DNA. Because it is a mitochondrial disease, it is only inherited through the mother. Individuals who have lost their central vision are referred to as “affected.” Individuals who carry one of the mitochondrial mutations but do not experience vision loss are referred to as “carriers.” LHON is caused by mutations in the following genes: MT-NDI1, MT-ND4, MT-ND4L and MT-ND6. Vision loss occurs because the cells in the optic nerve die.

Most of the LHON causative mutations affect mitochondrial DNA (mtDNA) genes encoding for NADH dehydrogenase (ND) subunits of the respiratory chain complex I, leading to a subacute and catastrophic degeneration of retinal ganglion cells (RGCs) (with the final outcome of optic nerve atrophy). The majority of LHON cases are caused by one of the three mtDNA missense point mutations at positions m.3460G>A/MT-ND1, m. 11778G>A/MT-ND4 and m. 14484T>C/MT-ND6.

In this example, a 19-year-old male patient visits a healthcare provider and presents complains of sudden vision loss, particularly to his central vision. After diagnosis is confirmed, a physician conducts a genetic analysis to identify the gene responsible for the disease: a missense point mutations at positions m.3460G>A/MT-ND1.

In this example, allotopic expression is used to introduce in the nucleus a wild-type (i.e., healthy) copy of the gene mutated in the mitochondrial genome and import normal copies of the gene product into mitochondria from the cytosol. The gene is introduced into the patient via a modified adeno-associated virus gene therapy to complement the defective ND4 gene by the allotopic expression of the wild-type ND4 subunit from the nucleus followed by mitochondrial import of the protein product.

The vector is introduced by intravitreal injection (IVT). Within six months of treatment, the patient's vision has improved (i.e., to about 80% of normal). The physician conducts regular testing of the patient's vision.

### Methods of Use

Embodiments include methods of treating pathogenesis or defects in the mitochondrial respiratory chain or the oxidative phosphorylation system. The methods can include allotopic expression of a wild-type version of a mutated gene (e.g., a gene with a deleterious mutation). Embodiments also include a method for slowing or retarding aging processes in humans by allotopic expression of one or more genes in human cells. Embodiments also include methods of treating aging-associated diseases by allotopic expression of one or more genes in human cells. In embodiments, the gene is a mitochondrial gene.

Also included is the use of a gene for manufacturing a medicine for treating a disease affecting the mitochondrial respiratory chain or oxidative phosphorylation system.

In one embodiment, a method of allotopic expression disclosed herein expresses, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% of a wild-type gene product that is introduced in the cell nucleus (i.e., to replace a mutated gene from the mitochondrial genome). In other aspects of this embodiment, a method disclosed herein leads to expression of a gene product by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In one embodiment, a therapeutic disclosed herein is capable of reducing the signs/symptoms of a mitochondrial disease or senescence-associated disease or disorder by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a therapeutic is capable of reducing the number of signs/symptoms of a mitochondrial disease or a senescence-associated disease or disorder in an individual by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In one embodiment, a therapeutic disclosed herein is capable of reducing signs/symptoms in an individual suffering from a mitochondrial disease or a senescence-associated disease or disorder by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a therapeutic is capable of reducing signs/symptoms in an individual suffering from a mitochondrial disease or a senescence-associated disease or disorder by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In one embodiment, a therapeutic disclosed herein is capable of reducing signs/symptoms of aging in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a therapeutic is capable of reducing signs/symptoms of aging in a subject by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using ‘consisting of’ or ‘consisting essentially of’ language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

1.-26. (canceled)

27. The method of treating a subject with a mitochondrial deficiency, the method comprising steps of:
a) identifying a mitochondrial gene associated with the mitochondrial deficiency,
b) expressing a modified or wild-type version of the mitochondrial gene in nuclei of cells of the subject, and
c) importing a gene product of the modified or wild-type version of the mitochondrial gene into mitochondria in cells of the subject to alleviate the mitochondrial deficiency.

28. (canceled)

29. The method of claim 27, wherein the gene product is a subunit of an oxidative phosphorylation (OXPHOS) complex.

30. The method of claim 27, wherein the gene product is a subunit of NADH ubiquinone oxidoreductase (complex I), succinate dehydrogenase (complex II), ubiquinol-cytochrome c oxidoreductase (complex III, or cytochrome bc1 complex), cytochrome c oxidase (complex IV), or ATP synthase (complex V).

31. The method of claim 27, wherein the mitochondrial dysfunction deficiency causes one or more of seizure, ataxia, cortical blindness, dystonia, exercise intolerance, ophthalmoplegia, optic atrophy, cataracts, diabetic mellitus, short stature, cardiomyopathy, sensorineural hearing loss, kidney failure, blindness, hearing loss, movement disorders (ataxia), dementia, cardiovascular disease, muscle weakness, renal dysfunction and an endocrine disorder in the subject.

32. The method of claim 27, wherein the step of expressing a modified or wild-type version of the mitochondrial gene further comprises a step of incorporating a nucleotide sequence into the modified or wild-type gene for codon optimization.

33. The method of claim 27, wherein the step of expressing a modified or wild-type version of the mitochondrial gene further comprises a step of incorporating a nucleotide sequence into the modified or wild-type gene that causes translational slow-down or ribosome stalling.

34. The method of claim 27, wherein the step of expressing a modified or wild-type version of the mitochondrial gene further comprises a step of incorporating a PUMILIO binding site into the modified or wild-type version of the mitochondrial gene.

35. The method of claim 27, wherein the step of expressing a modified or wild-type version of the mitochondrial gene comprises a step incorporating a nucleotide sequence into the modified or wild-type gene that reduces hydrophobicity of the gene product.

36. The method of claim 27, wherein the step of expressing a modified or wild-type version of the mitochondrial gene comprises a step of incorporating one or more codon sequences into the wild-type version of the mitochondrial gene,
wherein the one or more codon sequences introduce amino acid changes to reduce hydrophobicity of the gene product, reduce a localized charge of the gene product or cause ribosome stalling.

37. The method of claim 27, wherein the step of expressing a modified or wild-type version of the mitochondrial gene further comprises a step of incorporating one or more codon sequences into the modified or wild-type version of the mitochondrial gene,
wherein the one or more codon sequences encode an N-terminal matrix export import signal into the gene product.

38. The method of claim 37, wherein the N-terminal matrix export import signal is a six-residue sequence from OXA1L.

39. The method of claim 27, wherein the step of expressing a modified or wild-type version of the mitochondrial gene further comprises a step of incorporating one or more codon sequences into the wild-type version of the mitochondrial gene,
wherein the one or more codon sequences restore natural orientation of transmembrane helices of the gene product into the mitochondrial inner membrane.

40. The method of claim 27, wherein the step of expressing a modified or wild-type version of the mitochondrial gene further comprises a step of incorporating one or more codon sequences into the wild-type version of the mitochondrial gene,
wherein the one or more codon sequences introduce amino acid changes to reduce or enhance a localized charge of the gene product.

41. The method of claim 27, wherein the step of expressing a modified or wild-type version of the mitochondrial gene further comprises a step of adding a promoter sequence to the modified or wild-type version of the mitochondrial gene,
wherein the promoter sequence is selected from pCAG, PGK, PGC1a, ER1a, Myosin and CK.

42. The method of claim 27, wherein the step of expressing a modified or wild-type version of the mitochondrial gene further comprises a step of incorporating one or more codon sequences into the modified or wild-type version of the mitochondrial gene,
wherein the one or more codon sequences encode a mitochondrial targeting sequence into an amino terminal portion of the gene product.

43. The method of claim 27, wherein the mitochondrial deficiency causes a senescence-associated disease or disorder in the subject,
wherein the senescence-associated disease or disorder is one or more of atherosclerosis, osteoarthritis, osteoporosis, hypertension, arthritis, cataracts, cancer, Alzheimer's disease, chronic obstructive pulmonary disease (COPD) and idiopathic pulmonary fibrosis, hair graying, sarcopenia, adiposity, neurogenesis, fibrosis and glaucoma.

44. (canceled)

45. The method of claim 27, wherein the modified or wild-type version of the mitochondrial gene is a nuclear-encoded mitochondrial protein of respiratory complex I, and wherein the modified or wild-type version of the mitochondrial gene is paired with a mitochondrial targeting sequence (MTS) selected from SEQ ID NO: 1-29.

46. The method of claim 27, wherein the modified or wild-type version of the mitochondrial gene is a nuclear-encoded mitochondrial protein of respiratory complex V, and wherein the modified or wild-type version of the mitochondrial gene is paired with a mitochondrial targeting sequence (MTS) selected from SEQ ID NO: 30-48.

47. The method of claim 27, wherein the modified or wild-type version of the mitochondrial gene is a nuclear-encoded mitochondrial protein of respiratory complex IV, and wherein the modified or wild-type version of the mitochondrial gene is paired with a mitochondrial targeting sequence (MTS) selected from SEQ ID NO: 49-56.

48. The method of claim 27, wherein the modified or wild-type version of the mitochondrial gene is a nuclear-encoded mitochondrial protein of respiratory complex III, and wherein the modified or wild-type version of the mitochondrial gene is paired with a mitochondrial targeting sequence (MTS) selected from SEQ ID NO: 57-63.

49. The method of claim 27, wherein the modified or wild-type version of the mitochondrial gene is a transmembrane domain-containing nuclear encoded protein of the inner membrane, and wherein the modified or wild-type version of the mitochondrial gene is paired with a mitochondrial targeting sequence (MTS) selected from SEQ ID NO: 64-118.

50. The method of claim 27, wherein the modified or wild-type version of the mitochondrial gene is a nuclear encoded protein of the inner membrane lacking transmembrane (TM) domains, and wherein the modified or wild-type version of the mitochondrial gene is paired with a mitochondrial targeting sequence (MTS) selected from SEQ ID NO: 119-280.

51. The method of claim 27, wherein the modified or wild-type version of the mitochondrial gene is expressed from the nucleus via genome integration, or expressed as a non-integrating vector, a cDNA or mRNA.

52. The method of claim 27, wherein a viral vector is used in the step of expressing a modified or wild-type version of the mitochondrial gene in nuclei of cells of the subject,
wherein the viral vector is a lentiviral vector, a herpes simplex virus (HSV) vector an adenoviral vector or an adeno-associated viral (AAV) vector.

53.-77. (canceled)


SEQUENCE LISTING (280 sequences)

SEQ ID NO: 1 - protein, 20 aa, synthetic construct
  REGION 1..20: note = MTS Sequence
MLATRRLLGWSLPARVSVRF

SEQ ID NO: 2 - protein, 23 aa, synthetic construct
  REGION 1..23: note = MTS Sequence
MLRIPVRKALVGLSKSPKGCVRT

SEQ ID NO: 3 - protein, 24 aa, synthetic construct
  REGION 1..24: note = MTS Sequence
MALVHKLLRGTYFLRKFSKPTSAL

SEQ ID NO: 4 - protein, 27 aa, synthetic construct
  REGION 1..27: note = MTS Sequence
MAPSALLRPLSRLLAPARLPSGPSVRS

SEQ ID NO: 5 - protein, 28 aa, synthetic construct
  REGION 1..28: note = MTS Sequence
MAAAMTFCRLLNRCGEAARSLPLGARCF

SEQ ID NO: 6 - protein, 28 aa, synthetic construct
  REGION 1..28: note = MTS Sequence
MAVARAGVLGVQWLQRASRNVMPLGART

SEQ ID NO: 7 - protein, 29 aa, synthetic construct
  REGION 1..29: note = MTS Sequence
MAAGLFGLSARRLLAAAATRGLPAARVRW

SEQ ID NO: 8 - protein, 32 aa, synthetic construct
  REGION 1..32: note = MTS Sequence
MFFSAALRARAAGLTAHWGRHVRNLHKTVMQN

SEQ ID NO: 9 - protein, 33 aa, synthetic construct
  REGION 1..33: note = MTS Sequence
MAALRALCGFRGVAAQVLRPGAGVRLPIQPSRG

SEQ ID NO: 10 - protein, 33 aa, synthetic construct
  REGION 1..33: note = MTS Sequence
MSALTRLASFARVGGRLFRSGCARTAGDGGVRH

SEQ ID NO: 11 - protein, 34 aa, synthetic construct
  REGION 1..34: note = MTS Sequence
MAAPCLLRQGRAGALKTMLQEAQVFRGLASTVSL

SEQ ID NO: 12 - protein, 34 aa, synthetic construct
  REGION 1..34: note = MTS Sequence
MRCLTTPMLLRALAQAARAGPPGGRSLHSSAVAA

SEQ ID NO: 13 - protein, 35 aa, synthetic construct
  REGION 1..35: note = MTS Sequence
MAAAAQSRVVRVLSMSRSAITAIATSVCHGPPCRQ

SEQ ID NO: 14 - protein, 35 aa, synthetic construct
  REGION 1..35: note = MTS Sequence
MALRLLKLAATSASARVVAAGAQRVRGIHSSVQCK

SEQ ID NO: 15 - protein, 36 aa, synthetic construct
  REGION 1..36: note = MTS Sequence
MAAAAVARLWWRGILGASALTRGTGRPSVLLLPVRR

SEQ ID NO: 16 - protein, 38 aa, synthetic construct
  REGION 1..38: note = MTS Sequence
MAVLSAPGLRGFRILGLRSSVGPAVQARGVHQSVATDG

SEQ ID NO: 17 - protein, 42 aa, synthetic construct
  REGION 1..42: note = MTS Sequence
MAAVSMSVVLRQTLWRRRAVAVAALSVSRVPTRSLRTSTWRL

SEQ ID NO: 18 - protein, 46 aa, synthetic construct
  REGION 1..46: note = MTS Sequence
MAAMSLLRRVSVTAVAALSGRPLGTRLGFGGFLTRGFPKAAAPVRH

SEQ ID NO: 19 - protein, 68 aa, synthetic construct
  REGION 1..68: note = MTS Sequence
MASRVLSAYVSRLPAAFAPLPRVRMLAVARPLSTALCSAGTQTRLGTLQPALVLAQVPGRVTQLCRQY

SEQ ID NO: 20 - protein, 130 aa, synthetic construct
  REGION 1..130: note = MTS Sequence
MVTWLYRFLPTSNMAAKLRSLLPPDLRLQFWLHARLQKCFLSRGCGSYCAGAKASPLPGKMAMGLMCGRRELLRLLQSGRRVHSVAGPSQWLGKPLTTRLLFPVAPCCCRPHYLFLAASGPRSLSTSAIS

SEQ ID NO: 21 - skipped

SEQ ID NO: 22 - protein, 18 aa, synthetic construct
  REGION 1..18: note = MTS Sequence
MICTFLRAVQYTEKLHRS

SEQ ID NO: 23 - protein, 36 aa, synthetic construct
  REGION 1..36: note = MTS sequence
MLRPAGLWRLCRRPWAARVPAENLGRREVTSGVSPR

SEQ ID NO: 24 - protein, 37 aa, synthetic construct
  REGION 1..37: note = MTS Sequence
MSGCGLFLRTTAAARACRGLVVSTANRRLLRTSPPVR

SEQ ID NO: 25 - protein, 38 aa, synthetic construct
  REGION 1..38: note = MTS Sequence
MGIWQRLLLFGGVSLRAGGGATAPLGGSRAMVCGRQLS

SEQ ID NO: 26 - protein, 44 aa, synthetic construct
  REGION 1..44: note = MTS Sequence
MAASAHGSVWGPLRLGIPGLCCRRPPLGLYARMRRLPGPEVSGR

SEQ ID NO: 27 - protein, 46 aa, synthetic construct
  REGION 1..46: note = MTS Sequence
MSVLLRSGLGPLCAVARAAIPFIWRGKYFSSGNEPAENPVTPMLRH

SEQ ID NO: 28 - protein, 48 aa, synthetic construct
  REGION 1..48: note = MTS Sequence
MSWVQATLLARGLCRAWGGTCGAALTGTSISQVPRRLPRGLHCSAAAH

SEQ ID NO: 29 - protein, 54 aa, synthetic construct
  REGION 1..54: note = MTS Sequence
MFRCGGLAAGALKQKLVPLVRTVCVRSPRQRNRLPGNLFQRWHVPLELQMTRQM

SEQ ID NO: 30 - protein, 22 aa, synthetic construct
  REGION 1..22: note = MTS Sequence
MLPAALLRRPGLGRLVRHARAY

SEQ ID NO: 31 - protein, 23 aa, synthetic construct
  REGION 1..23: note = MTS Sequence
MAAPAVSGLSRQVRCFSTSVVRP

SEQ ID NO: 32 - protein, 25 aa, synthetic construct
  REGION 1..25: note = MTS Sequence
MFSRAGVAGLSAWTLQPQWIQVRNM

SEQ ID NO: 33 - protein, 32 aa, synthetic construct
  REGION 1..32: note = MTS Sequence
MILQRLFRFSSVIRSAVSVHLRRNIGVTAVAF

SEQ ID NO: 34 - protein, 40 aa, synthetic construct
  REGION 1..40: note = MTS Sequence
MCCAVSEQRLTCADQMMPFGKISQQLCGVKKLPWSCDSRY

SEQ ID NO: 35 - protein, 42 aa, synthetic construct
  REGION 1..42: note = MTS Sequence
MLSRVVLSAAATAAPSLKNAAFLGPGVLQATRTFHTGQPHLV

SEQ ID NO: 36 - protein, 43 aa, synthetic construct
  REGION 1..43: note = MTS Sequence
MLSVRVAAAVVRALPRRAGLVSRNALGSSFIAARNFHASNTHL

SEQ ID NO: 37 - protein, 47 aa, synthetic construct
  REGION 1..47: note = MTS Sequence
MLGFVGRVAAAPASGALRRLTPSASLPPAQLLLRAAPTAVHPVRDYA

SEQ ID NO: 38 - protein, 61 aa, synthetic construct
  REGION 1..61: note = MTS Sequence
MQTAGALFISPALIRCCTRGLIRPVSASFLNSPVNSSKQPSYSNFPLQVARREFQTSVVSR

SEQ ID NO: 39 - protein, 66 aa, synthetic construct
  REGION 1..66: note = MTS Sequence
MFACSKFVSTPSLVKSTSQLLSRPLSAVVLKRPEILTDESLSSLAVSCPLTSLVSSRSFQTSAISR

SEQ ID NO: 40 - protein, 67 aa, synthetic construct
  REGION 1..67: note = MTS Sequence
MFACAKLACTPSLIRAGSRVAYRPISASVLSRPEASRTGEGSTVFNGAQNGVSQLIQREFQTSAISR

SEQ ID NO: 41 - protein, 22 aa, synthetic construct
  REGION 1..22: note = MTS Sequence
MLPAALLRRPGLGRLVRHARAY

SEQ ID NO: 42 - protein, 23 aa, synthetic construct
  REGION 1..23: note = MTS Sequence
MAAPAVSGLSRQVRCFSTSVVRP

SEQ ID NO: 43 - protein, 25 aa, synthetic construct
  REGION 1..25: note = MTS Sequence
MFSRAGVAGLSAWTLQPQWIQVRNM

SEQ ID NO: 44 - protein, 81 aa, synthetic construct
  REGION 1..81: note = MTS Sequence
MLFLALGSPWAVELPLCGRRTALCAAAALRGPRASVSRASSSSGPSGPVAGWSTGPSGAARLLRRPGRAQIPVYWEGYVRF

SEQ ID NO: 45 - protein, 22 aa, synthetic construct
  REGION 1..22: note = MTS Sequence
MLPAALLRRPGLGRLVRHARAY

SEQ ID NO: 46 - protein, 40 aa, synthetic construct
  REGION 1..40: note = MTS Sequence
MWRSCLRLRDGGRRLLNRPAGGPSASMSPGPTIPSPARAY

SEQ ID NO: 47 - protein, 57 aa, synthetic construct
  REGION 1..57: note = MTS Sequence
MAAVVVAAAGGAGPAVLQVAGLYRGLCAVRSRALGLGLVSPAQLRVFPVRPGSGRPE

SEQ ID NO: 48 - protein, 81 aa, synthetic construct; identical to SEQ ID NO: 44
  REGION 1..81: note = MTS Sequence

SEQ ID NO: 49 - skipped

SEQ ID NO: 50 - protein, 20 aa, synthetic construct
  REGION 1..20: note = MTS Sequence
MLCRLGGRWLRPLPALQLWA

SEQ ID NO: 51 - skipped

SEQ ID NO: 52 - protein, 18 aa, synthetic construct
  REGION 1..18: note = MTS Sequence
MICTFLRAVQYTEKLHRS

SEQ ID NO: 53 - protein, 22 aa, synthetic construct
  REGION 1..22: note = MTS Sequence
MSRSSKVVLGLSVLLTAATVAG

SEQ ID NO: 54 - protein, 39 aa, synthetic construct
  REGION 1..39: note = MTS Sequence
MLPCAAGARGRGAMVVLRAGKKTFLPPLCRAFACRGCQL

SEQ ID NO: 55 - protein, 41 aa, synthetic construct
  REGION 1..41: note = MTS Sequence
MLLLTRSPTAWHRLSQLKPRVLPGTLGGQALHLRSWLLSRQ

SEQ ID NO: 56 - protein, 67 aa, synthetic construct
  REGION 1..67: note = MTS Sequence
MAMLVLVPGRVMRPLGGQLWRFLPRGLEFWGPAEGTARVLLRQFCARQAEAWRASGRPGYCLGTRPL

SEQ ID NO: 57 - protein, 13 aa, synthetic construct
  REGION 1..13: note = MTS Sequence
MAASRYRRFLKLC

SEQ ID NO: 58 - protein, 13 aa, synthetic construct
  REGION 1..13: note = MTS Sequence
MGLEDEQKMLTES

SEQ ID NO: 59 - protein, 13 aa, synthetic construct
  REGION 1..13: note = MTS Sequence
MGLEDEQKMLTES

SEQ ID NO: 60 - protein, 14 aa, synthetic construct
  REGION 1..14: note = MTS Sequence
MKLLTRAGSFSRFY

SEQ ID NO: 61 - protein, 34 aa, synthetic construct
  REGION 1..34: note = MTS Sequence
MAASVVCRAATAGAQVLLRARRSPALLRTPALRS

SEQ ID NO: 62 - protein, 70 aa, synthetic construct
  REGION 1..70: note = MTS Sequence
MFRLLSWSLGRGFLRAAGRRCRGCSARLLPGLAGGPGPEVQVPPSRVAPHGRGPGLLPLLAALAWFSRPA

SEQ ID NO: 63 - protein, 84 aa, synthetic construct
  REGION 1..84: note = MTS Sequence
MAAAAASLRGVVLGPRGAGLPGARARGLLCSARPGQLPLRTPQAVALSSKSGLSRGRKVMLSALGMLAAGGAGLAMALHSAVSA

SEQ ID NO: 64 - protein, 22 aa, synthetic construct
  REGION 1..22: note = MTS Sequence
MALLAMHSWRWAAAAAAFEKRR

SEQ ID NO: 65 - protein, 105 aa, synthetic construct
  REGION 1..105: note = MTS Sequence
MRGPPAWPLRLLEPPSPAEPGRLLPVACVWAAASRVPGSLSPFTGLRPARLWGAGPALLWGVGAARRWRSGCRGGGPGASRGVLGLARLLGLWARGPGSCRCGAF

SEQ ID NO: 66 - protein, 38 aa, synthetic construct
  REGION 1..38: note = MTS Sequence
MAHRCLRLWGRGGCWPRGLQQLLVPGGVGPGEQPCLRT

SEQ ID NO: 67 - protein, 61 aa, synthetic construct; identical to SEQ ID NO: 38
  REGION 1..61: note = MTS Sequence

SEQ ID NO: 68 - protein, 66 aa, synthetic construct; identical to SEQ ID NO: 39
  REGION 1..66: note = MTS Sequence

SEQ ID NO: 69 - protein, 67 aa, synthetic construct; identical to SEQ ID NO: 40
  REGION 1..67: note = MTS Sequence

SEQ ID NO: 70 - protein, 29 aa, synthetic construct
  REGION 1..29: note = MTS Sequence
MAALLLRHVGRHCLRAHFSPQLCIRNAVP

SEQ ID NO: 71 - protein, 34 aa, synthetic construct
  REGION 1..34: note = MTS Sequence
MLGSRAAGFARGLRALALAWLPGWRGRSFALARA

SEQ ID NO: 72 - protein, 28 aa, synthetic construct
  REGION 1..28: note = MTS Sequence
MAASPHTLSSRLLTGCVGGSVWYLERRT

SEQ ID NO: 73 - protein, 28 aa, synthetic construct
  REGION 1..28: note = MTS Sequence
MGGLWRPGWRCVPFCGWRWIHPGSPTRA

SEQ ID NO: 74 - protein, 20 aa, synthetic construct
  REGION 1..20: note = MTS Sequence
MLCRLGGRWLRPLPALQLWA

SEQ ID NO: 75 - protein, 22 aa, synthetic construct
  REGION 1..22: note = MTS Sequence
MLATRVFSLVGKRAISTSVCVR

SEQ ID NO: 76 - protein, 24 aa, synthetic construct
  REGION 1..24: note = MTS Sequence
MFPLVKSALNRLQVRSIQQTMARQ

SEQ ID NO: 77 - protein, 16 aa, synthetic construct
  REGION 1..16: note = MTS Sequence
MLGQSIRRFTTSVVRR

SEQ ID NO: 78 - protein, 25 aa, synthetic construct
  REGION 1..25: note = MTS Sequence
MSVLTPLLLRGLTGSARRLPVPRAK

SEQ ID NO: 79 - protein, 24 aa, synthetic construct
  REGION 1..24: note = MTS Sequence
MAVVGVSSVSRLLGRSRPQLGRPM

SEQ ID NO: 80 - protein, 84 aa, synthetic construct; identical to SEQ ID NO: 63
  REGION 1..84: note = MTS Sequence

SEQ ID NO: 81 - protein, 56 aa, synthetic construct
  REGION 1..56: note = MTS Sequence
MAVLWRLSAVCGALGGRALLLRTPVVRPAHISAFLQDRPIPEWCGVQHIHLSPSHH

SEQ ID NO: 82 - protein, 38 aa, synthetic construct
  REGION 1..38: note = MTS Sequence
MAAMRWRWWQRLLPWRLLQARGFPQNSAPSLGLGARTY

SEQ ID NO: 83 - protein, 52 aa, synthetic construct
  REGION 1..52: note = MTS Sequence
MASGAARWLVLAPVRSGALRSGPSLRKDGDVSAAWSGSGRSLVPSRSVIVTR

SEQ ID NO: 84 - protein, 45 aa, synthetic construct
  REGION 1..45: note = MTS Sequence
MLAARLVCLRTLPSRVFHPAFTKASPVVKNSITKNQWLLTPSREY

SEQ ID NO: 85 - protein, 115 aa, synthetic construct
  REGION 1..115: note = MTS Sequence
MASILLRSCRGRAPARLPPPPRYTVPRGSPGDPAHLSCASTLGLRNCLNVPFGCCTPIHPVYTSSRGDHLGCWALRPECLRIVSRAPWTSTSVGFVAVGPQCLPVRGWHSSRPVR

SEQ ID NO: 86 - protein, 25 aa, synthetic construct
  REGION 1..25: note = MTS Sequence
MAFYSYNSVLAIARTRFPSHFVHPT

SEQ ID NO: 87 - protein, 50 aa, synthetic construct
  REGION 1..50: note = MTS Sequence
MAAAAGRSLLLLLSSRGGGGGGAGGCGALTAGCFPGLGVSRHRQQQHHRT

SEQ ID NO: 88 - protein, 35 aa, synthetic construct
  REGION 1..35: note = MTS Sequence
MLQRGLWPWRTRLLPTPGTWRPARPWPLPPPPQVL

SEQ ID NO: 89 - protein, 27 aa, synthetic construct
  REGION 1..27: note = MTS Sequence
MAAIRMGKLTTMPAGLIYASVSVHAAK

SEQ ID NO: 90 - protein, 33 aa, synthetic construct
  REGION 1..33: note = MTS Sequence
MLRACQLSGVTAAAQSCLCGKFVLRPLRPCRRY

SEQ ID NO: 91 - protein, 33 aa, synthetic construct
  REGION 1..33: note = MTS Sequence
MFRLNSLSALAELAVGSRWYHGGSQPIQIRRRL

SEQ ID NO: 92 - protein, 35 aa, synthetic construct
  REGION 1..35: note = MTS Sequence
MMGRSPGFAMQHIVGVPHVLVRRGLLGRDLFMTRT

SEQ ID NO: 93 - protein, 25 aa, synthetic construct
  REGION 1..25: note = MTS Sequence
MLVHLFRVGIRGGPFPGRLLPPLRF

SEQ ID NO: 94 - protein, 49 aa, synthetic construct
  REGION 1..49: note = MTS Sequence
MFSSVAHLARANPFNTPHLQLVHDGLGDLRSSSPGPTGQPRRPRNLAAA

SEQ ID NO: 95 - protein, 49 aa, synthetic construct
  REGION 1..49: note = MTS Sequence
MECLRSLPCLLPRAMRLPRRTLCALALDVTSVGPPVAACGRRANLIGRS

SEQ ID NO: 96 - protein, 26 aa, synthetic construct
  REGION 1..26: note = MTS Sequence
MAGRRLNLRWALSVLCVLLMAETVSG

SEQ ID NO: 97 - protein, 46 aa, synthetic construct; identical to SEQ ID NO: 18
  REGION 1..46: note = MTS Sequence

SEQ ID NO: 98 - protein, 28 aa, synthetic construct
  REGION 1..28: note = MTS Sequence
MAVARAGVLGVQWLQRASRNVMPLGART

SEQ ID NO: 99 - protein, 29 aa, synthetic construct
  REGION 1..29: note = MTS Sequence
MAAGLFGLSARRLLAAAATRGLPAARVRW

SEQ ID NO: 100 - protein, 27 aa, synthetic construct
  REGION 1..27: note = MTS Sequence
MAPSALLRPLSRLLAPARLPSGPSVRS

SEQ ID NO: 101 - protein, 43 aa, synthetic construct
  REGION 1..43: note = MTS Sequence
MANLLKTVVTGCSCPLLSNLGSCKGLRVKKDFLRTFYTHQELW

SEQ ID NO: 102 - protein, 13 aa, synthetic construct
  REGION 1..13: note = MTS Sequence
MSFICGLQSAARN

SEQ ID NO: 103 - protein, 87 aa, synthetic construct
  REGION 1..87: note = MTS Sequence
MWRLRRAAVACEVCQSLVKHSSGIKGSLPLQKLHLVSRSIYHSHHPTLKLQRPQLRTSFQQFSSLTNLPLRKLKFSPIKYGYQPRRN

SEQ ID NO: 104 - protein, 72 aa, synthetic construct
  REGION 1..72: note = MTS Sequence
MAMGLMCGRRELLRLLQSGRRVHSVAGPSQWLGKPLTTRLLFPAAPCCCRPHYLFLAASGPRSLSTSAISFA

SEQ ID NO: 105 - protein, 52 aa, synthetic construct
  REGION 1..52: note = MTS Sequence
MAWRGWAQRGWGCGQAWGASVGGRSCEELTAVLTPPQLLGRRFNFFIQQKCG

SEQ ID NO: 106 - protein, 77 aa, synthetic construct
  REGION 1..77: note = MTS Sequence
MAVRQALGRGLQLGRALLLRFTGKPGRAYGLGRPGPAAGCVRGERPGWAAGPGAEPRRVGLGLPNRLRFFRQSVAGL

SEQ ID NO: 107 - protein, 52 aa, synthetic construct
  REGION 1..52: note = MTS Sequence
MATSVGHRCLGLLHGVAPWRSSLHPCEITALSQSLQPLRKLPFRAFRTDARK

SEQ ID NO: 108 - skipped

SEQ ID NO: 109 - protein, 10 aa, synthetic construct
  REGION 1..10: note = MTS Sequence
MAWRHLKKRA

SEQ ID NO: 110 - protein, 67 aa, synthetic construct; identical to SEQ ID NO: 56
  REGION 1..67: note = MTS Sequence

SEQ ID NO: 111 - protein, 41 aa, synthetic construct; identical to SEQ ID NO: 55
  REGION 1..41: note = MTS Sequence

SEQ ID NO: 112 - protein, 43 aa, synthetic construct
  REGION 1..43: note = MTS Sequence
MAVLLLLLRALRRGPGPGPRPLWGPGPAWSPGFPARPGRGRPY

SEQ ID NO: 113 - protein, 18 aa, synthetic construct
  REGION 1..18: note = MTS Sequence
MICTFLRAVQYTEKLHRS

SEQ ID NO: 114 - protein, 31 aa, synthetic construct
  REGION 1..31: note = MTS Sequence
MAAAALRRFWSRRRAEAGDAVVAKPGVWARL

SEQ ID NO: 115 - protein, 44 aa, synthetic construct
  REGION 1..44: note = MTS Sequence
MAASAAVFSRLRSGLRLGSRGLCTRLATPPRRAPDQAAEIGSRG

SEQ ID NO: 116 - protein, 61 aa, synthetic construct
  REGION 1..61: note = MTS Sequence
MSRLLPLLRSRTARSLRPGPAAAAAPRPPSWCCCGRGLLALAPPGGLPGGPRRLGTHPKKE

SEQ ID NO: 117 - protein, 81 aa, synthetic construct; identical to SEQ ID NO: 44
  REGION 1..81: note = MTS Sequence

SEQ ID NO: 118 - protein, 13 aa, synthetic construct
  REGION 1..13: note = MTS Sequence
MPAPRAPRALAAA

SEQ ID NO: 119 - protein, 37 aa, synthetic construct; identical to SEQ ID NO: 24
  REGION 1..37: note = MTS Sequence

SEQ ID NO: 120 - protein, 68 aa, synthetic construct; identical to SEQ ID NO: 19
  REGION 1..68: note = MTS Sequence

SEQ ID NO: 121 - protein, 32 aa, synthetic construct
  REGION 1..32: note = MTS Sequence
MASRCWRWWGWSAWPRTRLPPAGSTPSFCHHF

SEQ ID NO: 122 - protein, 54 aa, synthetic construct; identical to SEQ ID NO: 29
  REGION 1..54: note = MTS Sequence

SEQ ID NO: 123 - protein, 42 aa, synthetic construct; identical to SEQ ID NO: 35
  REGION 1..42: note = MTS Sequence

SEQ ID NO: 124 - protein, 32 aa, synthetic construct; identical to SEQ ID NO: 33
  REGION 1..32: note = MTS Sequence

SEQ ID NO: 125 - protein, 40 aa, synthetic construct; identical to SEQ ID NO: 34
  REGION 1..40: note = MTS Sequence

SEQ ID NO: 126 - protein, 43 aa, synthetic construct; identical to SEQ ID NO: 36
  REGION 1..43: note = MTS Sequence

SEQ ID NO: 127 - protein, 47 aa, synthetic construct; identical to SEQ ID NO: 37
  REGION 1..47: note = MTS Sequence

SEQ ID NO: 128 - protein, 22 aa, synthetic construct
  REGION 1..22: note = MTS Sequence
MLPAALLRRPGLGRLVRHARAY

SEQ ID NO: 129 - protein, 25 aa, synthetic construct
  REGION 1..25: note = MTS Sequence
MFSRAGVAGLSAWTLQPQWIQVRNM

SEQ ID NO: 130 - protein, 23 aa, synthetic construct
  REGION 1..23: note = MTS Sequence
MAAPAVSGLSRQVRCFSTSVVRP

SEQ ID NO: 131 - protein, 24 aa, synthetic construct
  REGION 1..24: note = MTS Sequence
MALRAKAEVCMAVPWLSLQRAQAL

SEQ ID NO: 132 - protein, 24 aa, synthetic construct
  REGION 1..24: note = MTS Sequence
MALRAKAEVCVAAPWLSLQRARAL

SEQ ID NO: 133 - protein, 47 aa, synthetic construct
  REGION 1..47: note = MTS Sequence
MMRRTLENRNAQTKQLQTAVSNVEKHFGELCQIFAAYVRKTARLRDK

SEQ ID NO: 134 - protein, 26 aa, synthetic construct
  REGION 1..26: note = MTS Sequence
MLRLPTVFRQMRPVSRVLAPHLTRAY

SEQ ID NO: 135 - protein, 29 aa, synthetic construct
  REGION 1..29: note = MTS Sequence
MWCLLRGLGRPGALARGALGQQQSLGARA

SEQ ID NO: 136 - protein, 24 aa, synthetic construct
  REGION 1..24: note = MTS Sequence
MALVHKLLRGTYFLRKFSKPTSAL

SEQ ID NO: 137 - protein, 56 aa, synthetic construct
  REGION 1..56: note = MTS Sequence
MPSCGACTCGAAAVRLITSSLASAQRGISGGRIHMSVLGRLGTFETQILQRAPLRS

SEQ ID NO: 138 - protein, 85 aa, synthetic construct
  REGION 1..85: note = MTS Sequence
MWSGRKLGSSGGWFLRVLGPGGCNTKAARPLISSAVYVKNQLSGTLQIKPGVFNEYRTIWFKSYRTIFSCLNRIKSFRYPWARLY

SEQ ID NO: 139 - protein, 30 aa, synthetic construct
  REGION 1..30: note = MTS Sequence
MATLLRPVLRRLCGLPGLQRPAAEMPLRAR

SEQ ID NO: 140 - protein, 42 aa, synthetic construct
  REGION 1..42: note = MTS Sequence
MAAPGSCALWSYCGRGWSRAMRGCQLLGLRSSWPGDLLSARL

SEQ ID NO: 141 - protein, 28 aa, synthetic construct
  REGION 1..28: note = MTS Sequence
MAARLVSRCGAVRAAPHSGPLVSWRRWS

SEQ ID NO: 142 - protein, 35 aa, synthetic construct
  REGION 1..35: note = MTS Sequence
MSCAGAAAAPRLWRLRPGARRSLSAYGRRTSVRFR

SEQ ID NO: 143 - protein, 44 aa, synthetic construct
  REGION 1..44: note = MTS Sequence
MAAAAVSGALGRAGWRLLQLRCLPVARCRQALVPRAFHASAVGL

SEQ ID NO: 144 - protein, 41 aa, synthetic construct
  REGION 1..41: note = MTS Sequence
MLGAALRRCAVAATTRADPRGLLHSARTPGPAVAIQSVRCY

SEQ ID NO: 145 - protein, 31 aa, synthetic construct
  REGION 1..31: note = MTS Sequence
MASRLLRGAGTLAAQALRARGPSGAAAMRSM

SEQ ID NO: 146 - protein, 55 aa, synthetic construct
  REGION 1..55: note = MTS Sequence
MYYKFSGFTQKLAGAWASEAYSPQGLKPVVSTEAPPIIFATPTKLTSDSTVYDYA

SEQ ID NO: 147 - protein, 39 aa, synthetic construct
  REGION 1..39: note = MTS Sequence
MLAKGLPPRSVLVKGCQTFLSAPREGLGRLRVPTGEGAG

SEQ ID NO: 148 - protein, 35 aa, synthetic construct
  REGION 1..35: note = MTS Sequence
MSSPISKSRSLAAFLQQLRSPRQPPRLVTSTAYTS

SEQ ID NO: 149 - protein, 33 aa, synthetic construct
  REGION 1..33: note = MTS Sequence
MAALGCARLRWALRGAGRGLCPHGARAKAAIPA

SEQ ID NO: 150 - protein, 38 aa, synthetic construct
  REGION 1..38: note = MTS Sequence
MTRILTAFKVVRTLKTGFGFTNVTAHQKWKFSRPGIRL

SEQ ID NO: 151 - protein, 25 aa, synthetic construct
  REGION 1..25: note = MTS Sequence
MVPRLLLRAWPRGPAVGPGAPSRPL

SEQ ID NO: 152 - protein, 15 aa, synthetic construct
  REGION 1..15: note = MTS Sequence
MAWAGSRRVPAGTRA

SEQ ID NO: 153 - protein, 37 aa, synthetic construct
  REGION 1..37: note = MTS Sequence
MAARTGHTALRRVVSGCRPKSATAAGAQAPVRNGRYL

SEQ ID NO: 154 - protein, 101 aa, synthetic construct
  REGION 1..101: note = MTS Sequence
MWRLPGLLGRALPRTLGPSLWRVTPKSTSPDGPQTTSSTLLVPVPNLDRSGPHGPGTSGGPRSHGWKDAFQWMSSRVSPNTLWDAISWGTLAVLALQLARQ

SEQ ID NO: 155 - protein, 36 aa, synthetic construct
  REGION 1..36: note = MTS Sequence
MVACRAIGILSRFSAFRILRSRGYICRNFTGSSALL

SEQ ID NO: 156 - protein, 33 aa, synthetic construct
  REGION 1..33: note = MTS Sequence
MTILTYPFKNLPTASKWALRFSIRPLSCSSQLR

SEQ ID NO: 157 - protein, 48 aa, synthetic construct; identical to SEQ ID NO: 28
  REGION 1..48: note = MTS Sequence

SEQ ID NO: 158 - protein, 43 aa, synthetic construct
  REGION 1..43: note = MTS Sequence
MAAPSWRGARLVQSVLRVWQVGPHVARERVIPFSSLLGFQRRC

SEQ ID NO: 159 - protein, 33 aa, synthetic construct
  REGION 1..33: note = MTS Sequence
MLVPLAKLSCLAYQCFHALKIKKNYLPLCATRW

SEQ ID NO: 160 - protein, 41 aa, synthetic construct
  REGION 1..41: note = MTS Sequence
MAIKSIASRLRGSRRFLSGFVAGAVVGAAGAGLAALQFFRS

SEQ ID NO: 161 - protein, 42 aa, synthetic construct
  REGION 1..42: note = MTS Sequence
MSRARSHLRAALFLAAASARGITTQVAARRGLSAWPVPQEPS

SEQ ID NO: 162 - protein, 37 aa, synthetic construct
  REGION 1..37: note = MTS Sequence
MLRVRCLRGGSRGAEAVHYIGSRLGRTLTGWVQRTFQ

SEQ ID NO: 163 - protein, 29 aa, synthetic construct
  REGION 1..29: note = MTS Sequence
MLYFSLFWAARPLQRCGQLVRMAIRAQHS

SEQ ID NO: 164 - protein, 42 aa, synthetic construct
  REGION 1..42: note = MTS Sequence
MAFQKAVKGTILVGGGALATVLGLSQFAHYRRKQMNLAYVKA

SEQ ID NO: 165 - protein, 46 aa, synthetic construct
  REGION 1..46: note = MTS Sequence
MISASRAAAARLVGAAASRGPTAARHQDSWNGLSHEAFRLVSRRDY

SEQ ID NO: 166 - protein, 27 aa, synthetic construct
  REGION 1..27: note = MTS Sequence
MAAQCVRLARRSLPALALSLRPSPRLL

SEQ ID NO: 167 - protein, 32 aa, synthetic construct
  REGION 1..32: note = MTS Sequence
MAVRSLWAGRLRVQRLLAWSAAWESKGWPLPF

SEQ ID NO: 168 - protein, 49 aa, synthetic construct
  REGION 1..49: note = MTS Sequence
MWTLVGRGWGCARALAPRATGAALLVAPGPRSAPTLGAAPESWATDRLY

SEQ ID NO: 169 - protein, 49 aa, synthetic construct
  REGION 1..49: note = MTS Sequence
MVTAAMLLQCCPVLARGPTSLLGKVVKTHQFLFGIGRCPILATQGPNCS

SEQ ID NO: 170 - protein, 56 aa, synthetic construct
  REGION 1..56: note = MTS Sequence
MESVVRRCPFLSRVPQAFLQKAGKSLLFYAQNCPKMMEVGAKPAPRALSTAAVHYQ

SEQ ID NO: 171 - protein, 110 aa, synthetic construct
  REGION 1..110: note = MTS Sequence
MALQLGRLSSGPCWLVARGGCGGPRAWSQCGGGGLRAWSQRSAAGRVCRPPGPAGTEQSRGLGHGSTSRGGPWVGTGLAAALAGLVGLATAAFGHVQRAEMLPKTSGTRA

SEQ ID NO: 172 - protein, 54 aa, synthetic construct
  REGION 1..54: note = MTS Sequence
MRSLGANMAAALRAAGVLLRDPLASSSWRVCQPWRWKSGAAAAAVTTETAQHAQ

SEQ ID NO: 173 - protein, 29 aa, synthetic construct
  REGION 1..29: note = MTS Sequence
MAATRCLRWGLSRAGVWLLPPPARCPRRA

SEQ ID NO: 174 - protein, 21 aa, synthetic construct
  REGION 1..21: note = MTS Sequence
MWPGNAWRAALFWVPRGRRAQ

SEQ ID NO: 175 - protein, 39 aa, synthetic construct
  REGION 1..39: note = MTS Sequence
MASIFSKLLTGRNASLLFATMGTSVLTTGYLLNRQKVCA

SEQ ID NO: 176 - protein, 39 aa, synthetic construct
  REGION 1..39: note = MTS Sequence
MAGPFSRLLSARPGLRLLALAGAGSLAAGFLLRPEPVRA

SEQ ID NO: 177 - protein, 51 aa, synthetic construct
  REGION 1..51: note = MTS Sequence
MVPALRYLVGACGRARGLFAGGSPGACGFASGRPRPLCGGSRSASTSSFDI

SEQ ID NO: 178 - protein, 52 aa, synthetic construct
  REGION 1..52: note = MTS Sequence
MARLLRSATWELFPWRGYCSQKAKGELCRDFVEALKAVVGGSHVSTAAVVRE

SEQ ID NO: 179 - protein, 96 aa, synthetic construct
  REGION 1..96: note = MTS Sequence
MALAARLLPQFLHSRSLPCGAVRLRTPAVAEVRLPSATLCYFCRCRLGLGAALFPRSARALAASALPAQGSRWPVLSSPGLPAAFASFPACPQRSY

SEQ ID NO: 180 - protein, 22 aa, synthetic construct
  REGION 1..22: note = MTS Sequence
MAAAAGSCARVAAWGGKLRRGL

SEQ ID NO: 181 - protein, 33 aa, synthetic construct
  REGION 1..33: note = MTS Sequence
MAAVVLAATRLLRGSGSWGCSRLRFGPPAYRRF

SEQ ID NO: 182 - protein, 43 aa, synthetic construct
  REGION 1..43: note = MTS Sequence
MAAAAARVVLSSAARRRLWGFSESLLIRGAAGRSLYFGENRLR

SEQ ID NO: 183 - protein, 8 aa, synthetic construct
  REGION 1..8: note = MTS Sequence
MRLTPRAL

SEQ ID NO: 184 - protein, 28 aa, synthetic construct
  REGION 1..28: note = MTS Sequence
MGGLFWRSALRGLRCGPRAPGPSLLVRH

SEQ ID NO: 185 - protein, 35 aa, synthetic construct; identical to SEQ ID NO: 13
  REGION 1..35: note = MTS Sequence

SEQ ID NO: 186 - protein, 35 aa, synthetic construct; identical to SEQ ID NO: 14
  REGION 1..35: note = MTS Sequence

SEQ ID NO: 187 - protein, 33 aa, synthetic construct; identical to SEQ ID NO: 10
  REGION 1..33: note = MTS Sequence

SEQ ID NO: 188 - skipped

SEQ ID NO: 189 - protein, 36 aa, synthetic construct; identical to SEQ ID NO: 23
  REGION 1..36: note = MTS Sequence

SEQ ID NO: 190 - protein, 44 aa, synthetic construct; identical to SEQ ID NO: 26
  REGION 1..44: note = MTS Sequence

SEQ ID NO: 191 - protein, 23 aa, synthetic construct
  REGION 1..23: note = MTS Sequence
MLRIPVRKALVGLSKSPKGCVRT

SEQ ID NO: 192 - protein, 33 aa, synthetic construct; identical to SEQ ID NO: 9
  REGION 1..33: note = MTS Sequence

SEQ ID NO: 193 - protein, 36 aa, synthetic construct; identical to SEQ ID NO: 15
  REGION 1..36: note = MTS Sequence

SEQ ID NO: 194 - protein, 42 aa, synthetic construct; identical to SEQ ID NO: 17
  REGION 1..42: note = MTS Sequence

SEQ ID NO: 195 - protein, 28 aa, synthetic construct
  REGION 1..28: note = MTS Sequence
MAAAMTFCRLLNRCGEAARSLPLGARCF

SEQ ID NO: 196 - protein, 38 aa, synthetic construct; identical to SEQ ID NO: 16
  REGION 1..38: note = MTS Sequence

SEQ ID NO: 197 - protein, 34 aa, synthetic construct; identical to SEQ ID NO: 12
  REGION 1..34: note = MTS Sequence

SEQ ID NO: 198 - protein, 20 aa, synthetic construct
  REGION 1..20: note = MTS Sequence
MLATRRLLGWSLPARVSVRF

SEQ ID NO: 199 - protein, 32 aa, synthetic construct; identical to SEQ ID NO: 8
  REGION 1..32: note = MTS Sequence

SEQ ID NO: 200 - protein, 34 aa, synthetic construct; identical to SEQ ID NO: 11
  REGION 1..34: note = MTS Sequence

SEQ ID NO: 201 - protein, 48 aa, synthetic construct
  REGION 1..48: note = MTS Sequence
MRALRAGLTLASGAGLGAVVEGWRRRREDARAAPGLLGRLPVLPVAAA

SEQ ID NO: 202 - protein, 32 aa, synthetic construct
  REGION 1..32: note = MTS Sequence
MLFNLRILLNNAAFRNGHNFMVRNFRCGQPLQ

SEQ ID NO: 203 - protein, 28 aa, synthetic construct
  REGION 1..28: note = MTS Sequence
MAVAAAAAAGPVFWRRLLGLLPGRPGLA

SEQ ID NO: 204 - protein, 29 aa, synthetic construct
  REGION 1..29: note = MTS Sequence
MLALRCGSRWLGLLSVPRSVPLRLPAARA

SEQ ID NO: 205 - skipped

SEQ ID NO: 206 - protein, 37 aa, synthetic construct
  REGION 1..37: note = MTS Sequence
MAVVSAVRWLGLRSRLGQPLTGRRAGLCEQARSCRFY

SEQ ID NO: 207 - protein, 27 aa, synthetic construct
  REGION 1..27: note = MTS Sequence
MAATALLEAGLARVLFYPTLLYTLFRG

SEQ ID NO: 208 - protein, 34 aa, synthetic construct; identical to SEQ ID NO: 61
  REGION 1..34: note = MTS Sequence

SEQ ID NO: 209 - protein, 14 aa, synthetic construct
  REGION 1..14: note = MTS Sequence
MKLLTRAGSFSRFY

SEQ ID NO: 210 - protein, 13 aa, synthetic construct
  REGION 1..13: note = MTS Sequence
MGLEDEQKMLTES

SEQ ID NO: 211 - protein, 31 aa, synthetic construct
  REGION 1..31: note = MTS Sequence
MALVALVAGARLGRRLSGPGLGRGHWTAAGR

SEQ ID NO: 212 - protein, 50 aa, synthetic construct
  REGION 1..50: note = MTS Sequence
MAAAVRCMGRALIHHQRHSLSKMVYQTSLCSCSVNIRVPNRHFAAATKSA

SEQ ID NO: 213 - protein, 60 aa, synthetic construct
  REGION 1..60: note = MTS Sequence
MALCALTRALRSLNLAPPTVAAPAPSLFPAAQMMNNGLLQQPSALMLLPCRPVLTSVALN

SEQ ID NO: 214 - protein, 40 aa, synthetic construct
  REGION 1..40: note = MTS Sequence
MPGWRLLTQVGAQVLGRLGDGLGAALGPGNRTHIWLFVRG

SEQ ID NO: 215 - protein, 52 aa, synthetic construct
  REGION 1..52: note = MTS Sequence
MAAPVVTAPGRALLRAGAGRLLRGGVQELLRPRHEGNAPDLACNFSLSQNRG

SEQ ID NO: 216 - protein, 28 aa, synthetic construct
  REGION 1..28: note = MTS Sequence
MAAAVAGMLRGGLLPQAGRLPTLQTVRY

SEQ ID NO: 217 - protein, 43 aa, synthetic construct
  REGION 1..43: note = MTS Sequence
MSKLGRAARGLRKPEVGGVIRAIVRAGLAMPGPPLGPVLGQRG

SEQ ID NO: 218 - protein, 45 aa, synthetic construct
  REGION 1..45: note = MTS Sequence
MLPAAARPLWGPCLGLRAAAFRLARRQVPCVCAVRHMRSSGHQRC

SEQ ID NO: 219 - protein, 30 aa, synthetic construct
  REGION 1..30: note = MTS Sequence
MAFFTGLWGPFTCVSRVLSHHCFSTTGSLS

SEQ ID NO: 220 - protein, 21 aa, synthetic construct
  REGION 1..21: note = MTS Sequence
MAGPLQGGGARALDLLRGLPR

SEQ ID NO: 221 - protein, 29 aa, synthetic construct
  REGION 1..29: note = MTS Sequence
MWRLLARASAPLLRVPLSDSWALLPASAG

SEQ ID NO: 222 - skipped

SEQ ID NO: 223 - protein, 21 aa, synthetic construct
  REGION 1..21: note = MTS Sequence
MALRSRFWGLFSVCRNPGCRF

SEQ ID NO: 224 - protein, 43 aa, synthetic construct
  REGION 1..43: note = MTS Sequence
MAACIAAGHWAAMGLGRSFQAARTLLPPPASIACRVHAGPVRQ

SEQ ID NO: 225 - protein, 30 aa, synthetic construct
  REGION 1..30: note = MTS Sequence
MVFLTAQLWLRNRVTDRYFRIQEVLKHARH

SEQ ID NO: 226 - protein, 39 aa, synthetic construct
  REGION 1..39: note = MTS Sequence
MAASSLTVTLGRLASACSHSILRPSGPGAASLWSASRRF

SEQ ID NO: 227 - protein, 40 aa, synthetic construct
  REGION 1..40: note = MTS Sequence
MAAAVLGQLGALWIHNLRSRGKLALGVLPQSYIHTSASLD

SEQ ID NO: 228 - protein, 9 aa, synthetic construct
  REGION 1..9: note = MTS Sequence
MRLSALLAL

SEQ ID NO: 229 - protein, 30 aa, synthetic construct
  REGION 1..30: note = MTS Sequence
MASVVLALRTRTAVTSLLSPTPATALAVRY

SEQ ID NO: 230 - protein, 55 aa, synthetic construct
  REGION 1..55: note = MTS Sequence
MPLHKYPVWLWKRLQLREGICSRLPGHYLRSLEEERTPTPVHYRPHGAKFKINPK

SEQ ID NO: 231 - protein, 34 aa, synthetic construct
  REGION 1..34: note = MTS Sequence
MAGILRLVVQWPPGRLQTVTKGVESLICTDWIRH

SEQ ID NO: 232 - protein, 55 aa, synthetic construct
  REGION 1..55: note = MTS Sequence
MAMAMLVLVVSPWSAARGVLRNYWERLLRKLPQSRPGFPSPPWGPALAVQGPAMF

SEQ ID NO: 233 - skipped

SEQ ID NO: 234 - protein, 46 aa, synthetic construct
  REGION 1..46: note = MTS Sequence
MAVLAGSLLGPTSRSAALLGGRWLQPRAWLGFPDAWGLPTPQQARG

SEQ ID NO: 235 - protein, 46 aa, synthetic construct
  REGION 1..46: note = MTS Sequence
MAASAFAGAVRAASGILRPLNILASSTYRNCVKNASLISALSTGRF

SEQ ID NO: 236 - protein, 36 aa, synthetic construct
  REGION 1..36: note = MTS Sequence
MANLFIRKMVNPLLYLSRHTVKPRALSTFLFGSIRG

SEQ ID NO: 237 - protein, 29 aa, synthetic construct
  REGION 1..29: note = MTS Sequence
MALASGPARRALAGSGQLGLGGFGAPRRG

SEQ ID NO: 238 - protein, 26 aa, synthetic construct
  REGION 1..26: note = MTS Sequence
MAAPWWRAALCECRRWRGFSTSAVLG

SEQ ID NO: 239 - protein, 46 aa, synthetic construct
  REGION 1..46: note = MTS Sequence
MTASVLRSISLALRPTSGLLGTWQTQLRETHQRASLLSFWELIPMR

SEQ ID NO: 240 - protein, 13 aa, synthetic construct
  REGION 1..13: note = MTS Sequence
MGVLAAAARCLVR

SEQ ID NO: 241 - protein, 32 aa, synthetic construct
  REGION 1..32: note = MTS Sequence
MAVAAVKWVMSKRTILKHLFPVQNGALYCVCH

SEQ ID NO: 242 - skipped

SEQ ID NO: 243 - protein, 30 aa, synthetic construct
  REGION 1..30: note = MTS Sequence
MASGLVRLLQQGHRCLLAPVAPKLVPPVRG

SEQ ID NO: 244 - protein, 20 aa, synthetic construct
  REGION 1..20: note = MTS Sequence
MRLFSYPTLFTLSSLPGGLC

SEQ ID NO: 245 - protein, 54 aa, synthetic construct
  REGION 1..54: note = MTS Sequence
MAAAPVRRTLLGVAGGWRRFERLWAGSLSSRSLALAAAPSSNGSPWRLLGALCL

SEQ ID NO: 246 - protein, 54 aa, synthetic construct
  REGION 1..54: note = MTS Sequence
MAAAGLALLCRRVSSALKSSRSLITPQVPACTGFFLSLLPKSTPNVTSFHQYRL

SEQ ID NO: 247 - protein, 28 aa, synthetic construct
  REGION 1..28: note = MTS Sequence
MSGTLEKVLCLRNNTIFKQAFSLLRFRT

SEQ ID NO: 248 - protein, 31 aa, synthetic construct
  REGION 1..31: note = MTS Sequence
MAGNLLSGAGRRLWDWVPLACRSFSLGVPRL

SEQ ID NO: 249 - protein, 23 aa, synthetic construct
  REGION 1..23: note = MTS Sequence
MAALGTVLFTGVRRLHCSVAAWA

SEQ ID NO: 250 - skipped

SEQ ID NO: 251 - protein, 14 aa, synthetic construct
  REGION 1..14: note = MTS Sequence
MATKRLFGATRTWA

SEQ ID NO: 252 - protein, 33 aa, synthetic construct
  REGION 1..33: note = MTS Sequence
MAAVGSLLGRLRQSTVKATGPALRRLHTSSWRA

SEQ ID NO: 253 - protein, 37 aa, synthetic construct
  REGION 1..37: note = MTS Sequence
MAAPAVKVARGWSGLALGVRRAVLQLPGLTQVRWSRY

SEQ ID NO: 254 - protein, 51 aa, synthetic construct
  REGION 1..51: note = MTS Sequence
MAAPCVSYGGAVSYRLLLWGRGSLARKQGLWKTAAPELQTNVRSQILRLRH

SEQ ID NO: 255 - protein, 39 aa, synthetic construct
  REGION 1..39: note = MTS Sequence
MQAVRNAGSRFLRSWTWPQTAGRVVARTPAGTICTGARQ

SEQ ID NO: 256 - protein, 29 aa, synthetic construct
  REGION 1..29: note = MTS Sequence
MSWSGLLHGLNTSLTCGPALVPRLWATCS

SEQ ID NO: 257 - protein, 57 aa, synthetic construct
  REGION 1..57: note = MTS Sequence
MLRVAWRTLSLIRTRAVTQVLVPGLPGGGSAKFPFNQWGLQPRSLLLQAARGYVVRK

SEQ ID NO: 258 - protein, 34 aa, synthetic construct
  REGION 1..34: note = MTS Sequence
MVHLTTLLCKAYRGGHLTIRLALGGCTNRPFYRI

SEQ ID NO: 259 - protein, 20 aa, synthetic construct
  REGION 1..20: note = MTS Sequence
MSVVRSSVHARWIVGKVIGT

SEQ ID NO: 260 - protein, 34 aa, synthetic construct
  REGION 1..34: note = MTS Sequence
MAALKALVSGCGRLLRGLLAGPAATSWSRLPARG

SEQ ID NO: 261 - protein, 35 aa, synthetic construct
  REGION 1..35: note = MTS Sequence
MAASVLNTVLRRLPMLSLFRGSHRVQVPLQTLCTK

SEQ ID NO: 262 - protein, 38 aa, synthetic construct
  REGION 1..38: note = MTS Sequence
MAVAVCGGLGRKKLTHLVTAAVSLTHPGTHTVLWRRGC

SEQ ID NO: 263 - protein, 35 aa, synthetic construct
  REGION 1..35: note = MTS Sequence
MAASVCSGLLGPRVLSWSRELPCAWRALHTSPVCA

SEQ ID NO: 264 - protein, 27 aa, synthetic construct
  REGION 1..27: note = MTS Sequence
MLRALSRLGAGTPCRPRAPLVLPARGR

SEQ ID NO: 265 - protein, 36 aa, synthetic construct
  REGION 1..36: note = MTS Sequence
MAASIVRRGMLLARQVVLPQLSPAGKRYLLSSAYVD

SEQ ID NO: 266 - protein, 71 aa, synthetic construct
  REGION 1..71: note = MTS Sequence
MAALCRTRAVAAESHFLRVFLFFRPFRGVGTESGSESGSSNAKEPKTRAGGFASALERHSELLQKVEPLQK

SEQ ID NO: 267 - protein, 21 aa, synthetic construct
  REGION 1..21: note = MTS Sequence
MMLKGITRLISRIHKLDPGRF

SEQ ID NO: 268 - protein, 65 aa, synthetic construct
  REGION 1..65: note = MTS Sequence
MFPRVSTFLPLRPLSRHPLSSGSPETSAAAIMLLTVRHGTVRYRSSALLARTKNNIQRYFGTNSV

SEQ ID NO: 269 - protein, 27 aa, synthetic construct
  REGION 1..27: note = MTS Sequence
MAAAALPAWLSLQSRARTLRAFSTAVY

SEQ ID NO: 270 - protein, 42 aa, synthetic construct
  REGION 1..42: note = MTS Sequence
MSGVRGLSRLLSARRLALAKAWPTVLQTGTRGFHFTVDGNKR

SEQ ID NO: 271 - protein, 28 aa, synthetic construct
  REGION 1..28: note = MTS Sequence
MAAVVALSLRRRLPATTLGGACLQASRG

SEQ ID NO: 272 - protein, 28 aa, synthetic construct
  REGION 1..28: note = MTS Sequence
MKMSFALTFRSAKGRWIANPSQPCSKAS

SEQ ID NO: 273 - protein, 38 aa, synthetic construct
  REGION 1..38: note = MTS Sequence
MVPLVAVVSGPRAQLFACLLRLGTQQVGPLQLHTGASH

SEQ ID NO: 274 - protein, 28 aa, synthetic construct
  REGION 1..28: note = MTS Sequence
MLARAARGTGALLLRGSLLASGRAPRRA

SEQ ID NO: 275 - skipped

SEQ ID NO: 276 - protein, 36 aa, synthetic construct
  REGION 1..36: note = MTS Sequence
MLRSCAARLRTLGALCLPPVGRRLPGSEPRPELRSF

SEQ ID NO: 277 - protein, 25 aa, synthetic construct
  REGION 1..25: note = MTS Sequence
MAAAALRSGWCRCPRRCLGSGIQFL

SEQ ID NO: 278 - protein, 59 aa, synthetic construct
  REGION 1..59: note = MTS Sequence
MARELRALLLWGRRLRPLLRAPALAAVPGGKPILCPRRTTAQLGPRRNPAWSLQAGRLF

SEQ ID NO: 279 - protein, 70 aa, synthetic construct; identical to SEQ ID NO: 62
  REGION 1..70: note = MTS Sequence

SEQ ID NO: 280 - protein, 13 aa, synthetic construct
  REGION 1..13: note = MTS Sequence
MAASRYRRFLKLC